(19) United States
(12) Patent Application Publication
(10) Pub. No.: US 2023/0348645 A1
(43) Pub. Date: Nov. 2, 2023

(54) IMPRINTED POLYMERS AND METHODS FOR THEIR USE

(71) Applicant: LIGAR LIMITED PARTNERSHIP, Hamilton (NZ)

(72) Inventors: Sujay KUMAR, Hamilton (NZ); Aaron Wai Kit LOW, Hamilton (NZ); Amber Hope Felicity MCELROY, Hamilton (NZ); Miao YU, Hamilton (NZ)

(73) Assignee: LIGAR LIMITED PARTNERSHIP, Hamilton (NZ)

(21) Appl. No.: 17/922,564

(22) PCT Filed: Apr. 29, 2021
(86) PCT No.: PCT/IB2021/053562

§ 371 Date: Oct. 31, 2022

(30) Foreign Application Priority Data
Apr. 29, 2020 (NZ) 763976

(51) Int. Cl. C08F 222/10; C08F 2/18; C08F 220/06; A61K 36/185

(52) U.S. Cl. CPC C08F 222/102; C08F 2/18; C08F 220/06; A61K 36/185; A61K 2236/30

(57) ABSTRACT

This disclosure relates to an imprinted polymer and/or an imprinted polymer bead for producing an enriched cannabinoid extract from a crude Cannabis extract. This disclosure further or alternatively relates to a method of making an imprinted polymer for producing an enriched cannabinoid extract from a crude Cannabis extract and/or a method of producing an enriched cannabinoid extract from a crude Cannabis extract and/or a method of reducing heavy metal content in an enriched cannabinoid extract when compared to a crude Cannabis extract and/or a method of reducing at least one pesticide residue in an enriched cannabinoid extract when compared to a crude Cannabis extract and/or a method of reducing lipid extraction steps when producing an enriched cannabinoid extract from a crude Cannabis extract and/or method of producing an enriched cannabinoid extract from a crude non-winterized Cannabis extract. This disclosure further relates to an enriched cannabinoid extract. More particularly, this disclosure relates to use of molecularly imprinted polymers to produce enriched cannabinoid extracts from crude Cannabis extract.

This disclosure relates to an imprinted polymer and/or an imprinted polymer bead for producing an enriched cannabinoid extract from a crude Cannabis extract. This disclosure further or alternatively relates to a method of making an imprinted polymer for producing an enriched cannabinoid extract from a crude Cannabis extract and/or a method of producing an enriched cannabinoid extract from a crude Cannabis extract and/or a method of reducing heavy metal content in an enriched cannabinoid extract when compared to a crude Cannabis extract and/or a method of reducing at least one pesticide residue in an enriched cannabinoid extract when compared to a crude Cannabis extract and/or a method of reducing lipid extraction steps when producing an enriched cannabinoid extract from a crude Cannabis extract and/or method of producing an enriched cannabinoid extract from a crude non-winterized Cannabis extract. This disclosure further relates to an enriched cannabinoid extract. More particularly, this disclosure relates to use of molecularly imprinted polymers to produce enriched cannabinoid extracts from crude Cannabis extract.

### BACKGROUND OF THE INVENTION

Many jurisdictions are lowering regulations around Cannabis production and sales, and this is driving increasing interest in uses for cannabis. A particular area of interest is Cannabis oils, which contain various compounds including cannabinoids and terpenoids or terpenes. Tetrahydrocannabinol (THC) is the primary psychoactive cannabinoid in cannabis. Cannabidiol (CBD) is a major constituent of Cannabis oil and is believed to have medicinal properties but is not believed to be psychoactive. Many different cannabinoids have been isolated from the Cannabis plant including tetrahydrocannabinolic acid (THCA), cannabidiolic acid (CBDA), cannabigerol (CBG), cannabinol (CBN) and cannabichromene (CBC). Many of these are also believed to have medicinal properties or are being studied. Many different terpenes have also been identified in Cannabis plants, including linalool, caryophyllene oxide, guaiol, alpha-bisabolol, beta-caryophyllene, delta-3-careen, beta-myrcene, D-limonene, alpha-humulene, trans-nerolidol, geraniol, valencene, terpineol, borneol, camphene, delta-3-carene, eucalyptol, alpha-pinene, beta-pinene and trans-nerolidol. These terpenes are believed to work in concert with cannabinoids to enhance their therapeutic effects.

Crude Cannabis extract is obtained from Cannabis plants by extraction of the plant matter with a solvent, for example supercritical carbon dioxide, an alcohol (for example ethanol) or hydrocarbon (for example, an alkane such as propane or butane). For example, the plant matter is soaked in ethanol, the plant material is then removed, the liquid filtered, and the ethanol removed by evaporation. These processes produce a crude extract (which can be an oily solid or viscous oil) that include waxes, fats, fatty acids, lipids, plant pigments, plant polyphenols and flavonoids, and in some cases heavy metal(s) and/or pesticide(s). The crude extract is generally bitter tasting and not palatable.

The crude extract undergoes further purification prior to use by a consumer. A key purification step is winterization, which is necessary for removing fat, wax and lipid impurities from Cannabis crude extracts. However, winterization is time consuming, energy-intensive and produces a significant bottleneck in the overall process.

Following winterization, further purification is carried out to enhance the levels of actives and/or remove further impurities. Current methods for further purification of crude extract include distillation or chromatography.

Distillation creates a bottleneck and involves heating the crude extract, often at high temperatures (for example 200-300° C.), which can convert acid forms of the cannabinoids into non-acid forms—thereby altering the naturally occurring cannabinoid profile extracted from the plant. Distillation can also result in loss of the naturally occurring terpenes. In order to obtain a product that contains the desirable terpenes, they must be recombined after distillation, which may not be considered natural or desirable. Distillation also results in concentrating any pesticide residue and heavy metals present in the crude extract, which can result in extracts that are not saleable under the regulatory regimes in some jurisdictions.

Chromatography is a complex process requiring expensive equipment and highly trained operators. The equipment gets exponentially more expensive as the volume to be processed increases, which creates throughput constraints for many producers.

Both distillation and chromatography can also result in significant reductions cannabinoid yield. It is believed as much as 30% of the cannabinoids present in a crude extract are lost through winterization and/or distillation and/or chromatography.

Overall, these multiple purification steps can negatively impact product yield and quality, the spectrum of actives in the product, process scalability, throughput and total productivity. In addition, the use of multiple purification steps to increase cannabinoid purity limits the ability to produce a ‘full spectrum’ or ‘broad spectrum’ product that retains the full or a broad spectrum of the actives that were present in the plant. Refined full/broad spectrum products are currently difficult to produce, the process often involving isolating individual active compounds then reformulating them to achieve the full/broad spectrum. Full/broad spectrum products currently fetch premium prices due to their therapeutic advantages over isolates. These therapeutic advantages are due to the ‘entourage effect’, which involves multiple compounds interacting synergistically to deliver a greater therapeutic effect than any one individual compound. There is also growing consumer awareness and distrust of the practice of recombining isolates in an attempt to form a full/broad spectrum-like product, and a section of consumers will prefer a more natural product that is not recombined or reconstituted.

In this specification, where reference has been made to external sources of information, including patent specifications and other documents, this is generally for the purpose of providing a context for discussing the features of the present invention. Unless stated otherwise, reference to such sources of information is not to be construed, in any jurisdiction, as an admission that such sources of information are prior art or form part of the common general knowledge in the art.

It is an object of this disclosure to provide an imprinted polymer for producing an enriched cannabinoid extract from a crude Cannabis extract and/or an imprinted polymer bead producing an enriched cannabinoid extract from a crude Cannabis extract and/or a method of making an imprinted polymer for producing an enriched cannabinoid extract from a crude Cannabis extract and/or a method of producing an enriched cannabinoid extract from a crude Cannabis extract and/or an enriched cannabinoid extract which goes at least some way towards overcoming one or more of the above mentioned problems or difficulties, or to at least provide the industry/public with a useful choice.

### SUMMARY OF THE INVENTION

In a first aspect there is provided an imprinted polymer for producing an enriched cannabinoid extract from a crude Cannabis extract, wherein the polymer is imprinted with a template organic molecule with a molecular weight of about 150 to 450 grams per mol comprising a hydroxyphenyl group, and wherein the polymer has been prepared from one or more polymerizable monomers.

In a second aspect there is provided an imprinted polymer bead for producing an enriched cannabinoid extract from a crude Cannabis extract, wherein the imprinted polymer bead is imprinted with a template organic molecule with a molecular weight of about 150 to 450 grams per mol comprising a hydroxyphenyl group, and wherein the imprinted polymer bead has been prepared from one or more polymerizable monomer(s).

In a third aspect there is provided a method of making an imprinted polymer for producing an enriched cannabinoid extract from a crude Cannabis extract, the method comprising polymerizing one or more polymerizable monomer(s) in the presence of a template organic molecule with a molecular weight of about 150 to 450 grams per mol comprising a hydroxyphenyl group, and optionally subsequently at least partially removing the template molecule from the imprinted polymer.

In a fourth aspect there is provided a method of producing an enriched cannabinoid extract from a crude Cannabis extract, the method comprising the steps of:
a) contacting an imprinted polymer with the crude Cannabis extract, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers;
b) eluting the cannabinoids from the imprinted polymer with an elution solvent to produce the enriched cannabinoid extract comprising one or more cannabinoid(s).

In a fifth aspect there is provided a method of reducing heavy metal content in an enriched cannabinoid extract when compared to a crude Cannabis extract, the method comprising the steps of:
a) contacting an imprinted polymer with the crude Cannabis extract, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers;
b) eluting the cannabinoids from the imprinted polymer with an elution solvent to produce the enriched cannabinoid extract comprising one or more cannabinoid(s).

In a sixth aspect there is provided a method of reducing at least one pesticide residue in an enriched cannabinoid extract when compared to a crude Cannabis extract, the method comprising the steps of:
a) contacting an imprinted polymer with the crude Cannabis extract, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers;
b) eluting the cannabinoids from the imprinted polymer with an elution solvent to produce the enriched cannabinoid extract comprising one or more cannabinoid(s).

In a seventh aspect there is provided a method of reducing lipid extraction steps when producing an enriched cannabinoid extract from a crude Cannabis extract, the method comprising the steps of:
a) contacting an imprinted polymer with the crude Cannabis extract containing lipid, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers;
b) eluting the cannabinoids from the imprinted polymer with an elution solvent to produce the enriched cannabinoid extract comprising one or more cannabinoid(s).

For the avoidance of doubt the following embodiments may apply alone or in any combination of two or more thereof to any one or more of the first, second, third, fourth, fifth, sixth and seventh aspects set forth above where the context allows.

In some embodiments the one or more polymerizable monomer(s) are selected from an acryl or a vinyl.

In some embodiments the acryl monomer is selected from:
acrylic acid, tert-butylacrylamide, N-phenylacrylamide, N-methylacrylamide, methacrylic acid, 2-(hydroxyethyl)methacrylate, ethylene glycol dimethacrylate (EGDMA), methacrylic anhydride or trimethylolpropane trimethacrylate.

In some embodiments the acryl monomer is a methacryl monomer.

In some embodiments the methacryl monomer is selected from methacrylic acid, 2-(hydroxyethyl)methacrylate, ethylene glycol dimethacrylate, methacrylic anhydride or trimethylolpropane trimethacrylate.

In some embodiments the vinyl monomer is selected from a styryl or vinylpyridine.

In some embodiments the vinylpyridine is 4-vinylpyridine.

In some embodiments the styryl monomer is selected from styrene, 4-vinylstyrene.

In some embodiments the polymer has been prepared from one or more acryls (including methacryl) monomers.

In some embodiments the polymer has been prepared from one or more styryl monomers.

In some embodiments the polymer is prepared from two or more monomers.

In some embodiments at least one of the monomers acts as a crosslinker.

In some embodiments the crosslinker is a diolefin.

In some embodiments the polymer is prepared from two or more styryl monomers.

In some embodiments the polymer has been prepared from divinylbenzene (DVB) and styrene monomers.

In some embodiments the ratio of divinylbenzene (DVB) and styrene is about 1:0.01-0.5 by molar mass. In some embodiments the ratio of divinylbenzene (DVB) and styrene is about 1:0.05-0.3 by molar mass. In some embodiments the ratio of divinylbenzene (DVB) and styrene is about 1:0.1-0.3 by molar mass.

In some embodiments the polymer is prepared from two or more acryl (including methacryl) monomers.

In some embodiments the polymer has been prepared from ethylene glycol dimethacrylate (EGDMA) and methacrylic acid monomers.

In some embodiments the ratio of ethylene glycol dimethacrylate (EGDMA) and methacrylic acid is about 1:0.1-2 by molar mass. In some embodiments the ratio of ethylene glycol dimethacrylate (EGDMA) and methacrylic acid is about 1:0.2-0.8 by molar mass. In some embodiments the ratio of ethylene glycol dimethacrylate (EGDMA) and methacrylic acid is about 1:0.4-0.5 by molar mass.

In some embodiments the polymer has been prepared from acrylic acid and ethylene glycol dimethacrylate (EGDMA) monomers.

In some embodiments the ratio of acrylic acid and ethylene glycol dimethacrylate (EGDMA) is about 1:0.2-1.1 by molar mass. In some embodiments the ratio of acrylic acid and ethylene glycol dimethacrylate (EGDMA) is about 1:0.4-0.8 by molar mass. In some embodiments the ratio of acrylic acid and ethylene glycol dimethacrylate (EGDMA) is about 1:0.6-0.7 by molar mass.

In some embodiments the polymer has been prepared from ethylene glycol dimethacrylate (EGDMA) and 2-(hydroxyethyl) methacrylate monomers.

In some embodiments the ratio of ethylene glycol dimethacrylate (EGDMA) and 2-(hydroxyethyl) methacrylate is about 1:1.5-0.05. In some embodiments the ratio of ethylene glycol dimethacrylate (EGDMA) and 2-(hydroxyethyl) methacrylate is about 1:1.5-0.1 by molar mass. In some embodiments the ratio of ethylene glycol dimethacrylate (EGDMA) and 2-(hydroxyethyl) methacrylate is about 1:1.2-0.8 by molar mass. In some embodiments the ratio of ethylene glycol dimethacrylate (EGDMA) and 2-(hydroxyethyl) methacrylate is about 1:0.5-0.1 by molar mass.

In some embodiments the polymer has been prepared from ethylene glycol dimethacrylate (EGDMA) and tert-butylacryamide (TBA) monomers.

In some embodiments the ratio of ethylene glycol dimethacrylate (EGDMA) and tert-butylacryamide (TBA) is about 1:0.05-0.5 by molar mass. In some embodiments the ratio of ethylene glycol dimethacrylate (EGDMA) and tert-butylacryamide (TBA) is about 1:0.05-0.3 by molar mass. In some embodiments the ratio of ethylene glycol dimethacrylate (EGDMA) and tert-butylacryamide (TBA) is about 1:0.1-0.2 by molar mass.

In some embodiments the polymer has been prepared from styrene and ethylene glycol dimethacrylate (EGDMA) monomers.

In some embodiments the ratio of ethylene glycol dimethacrylate (EGDMA) and styrene and is about 1:0.1-1 by molar mass. In some embodiments the ratio of ethylene glycol dimethacrylate (EGDMA) and styrene and is about 1:0.3-0.9 by molar mass. In some embodiments the ratio of ethylene glycol dimethacrylate (EGDMA) and styrene and is about 1:0.4-0.8 by molar mass. In some embodiments the ratio of ethylene glycol dimethacrylate (EGDMA) and styrene and is about 1:0.6-0.7 by molar mass.

In some embodiments the polymer has been prepared from ethylene glycol dimethacrylate (EGDMA) as the only monomer.

In some embodiments the polymer has been prepared from one or more crosslinking monomer(s) without non-crosslinking monomers.

In some embodiments the polymer has been prepared from one crosslinking monomer without non-crosslinking monomers.

In some embodiments the imprinted polymer is in the form of a bead.

In some embodiments the bead is about 0.1 to 10 mm in diameter. In some embodiments the bead is about 0.2 to 8 mm in diameter. In some embodiments the bead is about 0.2 to 6 mm in diameter. In some embodiments the bead is about 0.2 to 5 mm in diameter. In some embodiments the bead is about 0.2 to 6 mm in diameter. In some embodiments the bead is about 0.2 to 4 mm in diameter. In some embodiments the bead is about 0.2 to 3 mm in diameter. In some embodiments the bead is about 0.2 to 2 mm in diameter. In some embodiments the bead is about 0.5-2 mm diameter In some embodiments the bead is about 0.5 to 1.5 mm in diameter.

In some embodiments about 99% of the beads are about 0.1 to 10 mm in diameter. In some embodiments about 95% of the beads are about 0.1 to 10 mm in diameter. In some embodiments about 90% of the beads are about 0.1 to 10 mm in diameter In some embodiments about 85% of the beads are about 0.1 to 10 mm in diameter.

In some embodiments the bead has compression strength of about 300-13,800 psi.

In some embodiments the template molecule has a molecular weight of about 150 to 450 grams per mol (not including any associated salts or water molecules of hydration).

In some embodiments the template molecule has a molecular weight of about 150 to 450 grams per mol.

In some embodiments the template comprises the structure:

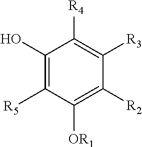

wherein R₁, R₂, R₃, R₄ and R₅ are the remainder of the organic molecule.

In some embodiments the template preferably comprises the structure:

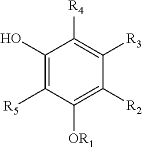

wherein R₁, R₂, R₃, R₄ and R₅ are the remainder of the organic molecule wherein
R₁ is H
R₂ is selected from H, or an organic group,
R₃ is selected from H, —OH, or an organic group,
R₄ is H or an organic group,
R₅ is H, or an organic group,
or R₁ and R₂ together form a 5- or 6-membered ring, preferably a 6-membered ring, optionally substituted with one or more of —OH, or an organic group, or
or R₁ and R₅ together form a 5- or 6-membered ring, preferably a 6-membered ring, optionally substituted with one or more substitutes independently selected from —OH, or an organic group, and wherein the 5- or 6-membered ring is optionally fused to a further ring which may be optionally substituted with one or more alkyl.

In some embodiments the template consists of C, H and O atoms.

In some embodiments one or more of the R₁, R₂, R₃, R₄ or R₅ groups is a saturated alkyl group, preferably a saturated alkyl group with 2 to 8 carbons.

In some embodiments at least one of R₂, R₃, and R₅ are an alkyl, preferably a 4-6 alkyl, or R₁ and R₂ or R₁ and R₅ together form the 5- or 6-membered ring.

In some embodiments R₃ is a C2-C8 saturated alkyl group, which may be branched or unbranched. In some embodiments R₃ is an unbranched pentyl group.

In some embodiments R₂ and R₃ or R₃ and R₄ or R₁ and R₂ or R₁ and R₅ form a fused ring, preferably a 5- or 6-membered ring, preferably a 6-membered ring wherein the fused ring is optionally substituted. In some embodiments the fused ring is substituted with a substituted or unsubstituted phenyl group. In some embodiments the phenyl group is substituted with one or more alcohol groups. In some embodiments the fused ring is substituted with an alcohol group.

In some embodiments:
R₁ is H
R₂ is selected from H, alkyl, or CORₓ, wherein Rₓ is alkyl, cycloalkyl or aryl, preferably aryl,
R₃ is selected from H, —OH or alkyl,
R₄ is H or alkyl,
R₅ is H, alkyl, or cycloalkyl optionally substituted with one or more alkyl or —OH,
or R₁ and R₂ together form a 5- or 6-membered ring, preferably a 6-membered ring, optionally substituted with one or more substituents independently selected from —OH, alkyl,
or aryl, wherein the aryl is optionally substituted with one or more —OH,
or R₁ and R₅ together form a 5- or 6-membered ring, preferably a 6-membered ring,
optionally substituted with one or more substituents independently selected from —OH, alkyl, or aryl, wherein the aryl is optionally substituted with one or more —OH, and wherein the 5- or 6-membered ring is optionally fused to a further 5- or 6-membered ring, preferably a cycloalkyl ring, which may be optionally substituted with one or more independently selected alkyl.

In some embodiments wherein R₂ is alkyl; and R₁, R₃, R₄, and R₅ are H.

In some embodiments wherein R₃ is alkyl; and R₁, R₂, R₄, and R₅ are H.

In some embodiments wherein R₂ is CORₓ wherein Rₓ is aryl, and R₁, R₃, R₄, and R₅ are H.

In some embodiments wherein R₃ and R₅ are each independently alkyl; and R₁, R₂, and R₄ are H.

In some embodiments wherein R₃ is alkyl; R₅ is cycloalkyl optionally substituted with one or more alkyl groups, and R₁, R₂, and R₄ are H.

In some embodiments wherein R₃ is OH; R₄ and R₅ are H; and R₁ and R₂ together form a 6-membered ring optionally substituted with one or more groups independently selected from —OH and phenyl, wherein the phenyl is optionally substituted with one or more —OH.

In some embodiments wherein R₃ is alkyl; R₂ and R₄ are H; and R₁ and R₅ together form a 6-membered ring optionally substituted with one or more substituents independently selected from —OH, and alkyl, and wherein the 6-membered ring is optionally fused to a further 6-membered ring which may be optionally substituted with one or more independently selected alkyl.

In some embodiments, the template is of the formula:

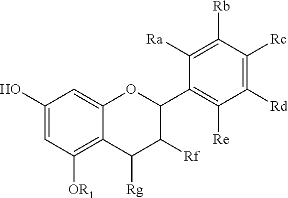

wherein Rₐ, R_{b}, R_{c}, R_{d}, Rₑ, R_{f} and R_{g} are optional substituents. Preferably Rₐ, R_{b}, R_{c}, R_{d}, Rₑ are independently —OH or —H. Preferably, R_{b}, R_{c} are —OH groups. Preferably R_{b}, R_{c} are —OH groups and Rₐ, R_{d} and Rₑ are hydrogen groups.

In some embodiments the template molecule is a cannabinoid.

In some embodiments the template molecule is selected from:

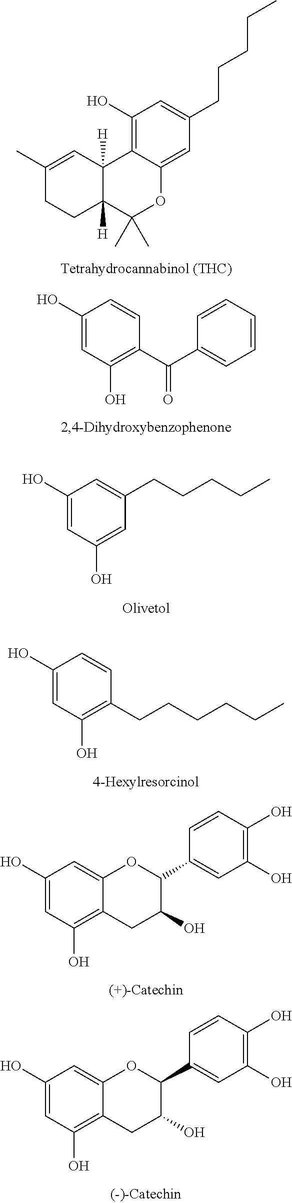

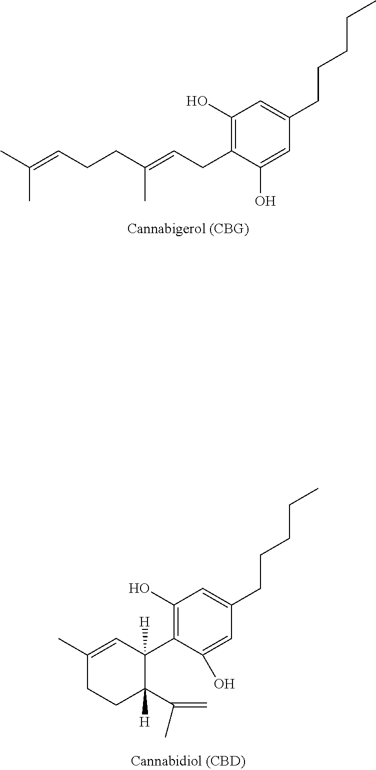

In some embodiments the template is selected from Cannabigerol (CBG), Cannabidiol (CBD), Tetrahydrocannabinol (THC).

In some embodiments the template is Cannabidiol (CBD).

In some embodiments the template is selected from:

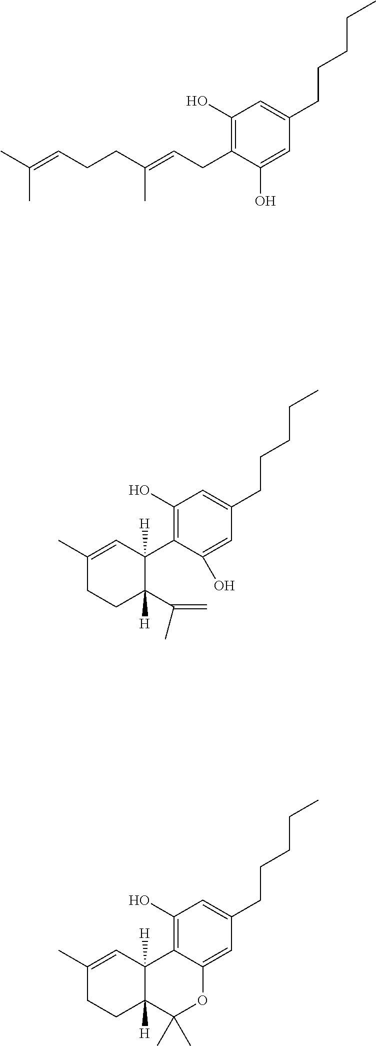

In some embodiments the template is:

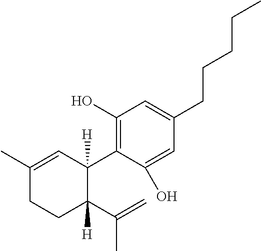 [SMILES: C=C(C)[C@@H]1CCC(C)=C[C@H]1c1c(O)cc(CCCCC)cc1O]

In some embodiments the template molecule is not a cannabinoid.

In some embodiments the template molecule is an alkyl- or acyl-resorcinol.

In some embodiments the template molecule is an 2,4 Dihydroxybenzophenone.

In some embodiments the template molecule is an olivetol.

In some embodiments the template molecule is an 4-Hexylresorcinol.

In some embodiments the template is a flavonoid or flavan.

In some embodiments the template molecule is a flavan-3-ol.

In some embodiments the template molecule is catechin.

In some embodiments the template molecule is (+/−)-catechin.

In some embodiments the ratio of the template to the one or more monomer(s) is about 1:10-300 by molar mass. In some embodiments the ratio of the template to the one or more monomer(s) is about 1:15-200 by molar mass. In some embodiments the ratio of the template to the one or more monomer(s) is about 1:20-160 by molar mass. In some embodiments the ratio of the template to the one or more monomer(s) is about 1:50-150 by molar mass. In some embodiments the ratio of the template to the one or more monomer(s) is about 1:70-120 by molar mass.

In some embodiments the method of making an imprinted polymer comprises polymerizing one or more monomers in the presence of an initiator.

In some embodiments the initiator is an oil-soluble azo initiator.

In some embodiments the oil-soluble azo initiator is selected from dimethyl 2,2′-azobis(2-methylpropionate), 2,2′-azobis(isobutyronitrile) (‘AIBN’), 2,2′-azobis(4-methoxy-2,4-dimethylvaleronitrile), 2,2′-azobis(2,4-dimethylvaleronitrile) or 2,2′-azobis (2-methylbutyronitrile).

In some embodiments the ratio of initiator to the one or more monomer(s) is about 0.001-0.2:1 by molar mass. In some embodiments the ratio of initiator to the one or more monomer(s) is about 0.01-0.15:1 by molar mass. In some embodiments the ratio of initiator to the one or more monomer(s) is about 0.01-0.1:1 by molar mass. In some embodiments the ratio of initiator to the one or more monomer(s) is about 0.01-0.05:1 by molar mass.

In some embodiments polymerizing is carried out in a suspension liquid.

In some embodiments the suspension liquid is selected from one or more of water and organic solvents such as mineral oil, perflurohydrocarbon, ethyl acetate, toluene, xylene, cyclohexane, hexane, benzene or heptane. In some embodiments the suspension liquid is water.

In some embodiments a monomer pre-mix solution which is immiscible with the suspension liquid comprises the one or more monomer(s), template and initiator is provided.

In some embodiments the monomer pre-mix solution comprises the one or more monomer(s), template and initiator which are dissolved in at least one solvent.

In some embodiments the monomer(s) and the template are dissolved in the solvent prior to addition of the initiator.

In some embodiments the monomer pre-mix solution is added to the suspension liquid.

In some embodiments the solvent is selected from one or more of ethyl acetate, toluene, xylene, cyclohexane, hexane, benzene, heptane, acetonitrile. In some embodiments the solvent is ethyl acetate. In some embodiments the solvent is acetonitrile.

In some embodiments the solvent is acetonitrile and the suspension liquid is mineral oil. In some embodiments the solvent is ethyl acetate and the suspension liquid is water.

In some embodiments the monomer pre-mix solution comprises acetonitrile, ethylene glycol dimethacrylate (EGDMA), the template and the initiator.

In some embodiments the monomer pre-mix solution comprises acetonitrile, methacrylic acid, ethylene glycol dimethacrylate (EGDMA), the template and the initiator.

In some embodiments the monomer pre-mix solution comprises acetonitrile, acrylic acid, ethylene glycol dimethacrylate (EGDMA), the template and the initiator.

In some embodiments the monomer pre-mix solution comprises acetonitrile, 2-(hydroxyethyl)methacrylate, ethylene glycol dimethacrylate (EGDMA), the template and the initiator.

In some embodiments the monomer pre-mix solution comprises ethyl acetate, divinylbenzene (DVB), styrene, the template and the initiator.

In some embodiments the monomer pre-mix solution comprises ethyl acetate, tert-butylacryamide (TBA), ethylene glycol dimethacrylate (EGDMA), the template and the initiator.

In some embodiments the monomer pre-mix solution comprises ethyl acetate, styrene, ethylene glycol dimethacrylate (EGDMA), the template and the initiator.

In some embodiments the ratio of suspension liquid and monomer pre-mix solution is about 0.5-80:1 by volume. In some embodiments the ratio of suspension liquid and monomer pre-mix solution is about 0.5-50:1 by volume.

In some embodiments the ratio of water as the suspension liquid and monomer pre-mix solution comprising ethyl acetate is about 100-10:1 by volume. In some embodiments the ratio of water as the suspension liquid and monomer pre-mix solution comprising ethyl acetate is about 20-60:1 by volume. In some embodiments the ratio of water as the suspension liquid and monomer pre-mix solution comprising ethyl acetate is about 30-50:1 by volume.

In some embodiments the ratio of mineral oil as the suspension liquid and monomer pre-mix solution comprising acetonitrile is about 0.5-20:1 by volume. In some embodiments the ratio of mineral oil as the suspension liquid and monomer pre-mix solution comprising acetonitrile is about 0.5-10:1 by volume. In some embodiments the ratio of mineral oil as the suspension liquid and monomer pre-mix solution comprising acetonitrile is about 0.6-8:1 by volume.

In some embodiments the liquid is agitated, such that the polymer forms in beads.

In some embodiments the liquid is agitated at about 100-1500 RPM. In some embodiments the liquid is agitated at about 200-1000 RPM.

In some embodiments the liquid is agitated for at least about 8 hours. In some embodiments the liquid is agitated for at least about 10 hours. In some embodiments the liquid is agitated for at least about 12 hours. In some embodiments the liquid is agitated for about 10-24 hours.

In some embodiments the polymerization is maintained at a temperature of between about 40-80° C. In some embodiments the polymerization is maintained at a temperature of between about 50-70° C.

In some embodiments the method of making an imprinted polymer comprises at least partially removing the template molecule from the imprinted polymer.

In some embodiments a solvent is used to at least partially remove the template molecule from the imprinted polymer.

In some embodiments following the method of the fourth, fifth, sixth or seventh aspects the enriched cannabinoid extract has a greater proportion of at least one cannabinoid than the crude Cannabis extract.

In some embodiments of the method of fourth, fifth, sixth or seventh aspects the enriched cannabinoid extract has an increase of about 5-30% of total cannabinoids by mass.

In some embodiments the enriched cannabinoid extract has an increased proportion of THC.

In some embodiments the enriched cannabinoid extract has an increased proportion of CBD.

In some embodiments the enriched cannabinoid extract has an increased proportion of CBG.

In some embodiments the enriched cannabinoid extract has an increased proportion of CBD and CBG.

In some embodiments the enriched cannabinoid extract comprises a cannabinoid in acid form.

In some embodiments the cannabinoid in acid form is THCA and/or CBDA.

In some embodiments the enriched cannabinoid extract has a reduced proportion of one or more heavy metals and/or pesticides compared to the crude Cannabis extract.

In some embodiments the one or more heavy metal(s) include arsenic, cadmium, chromium, copper, lead, nickel and/or zinc.

In some embodiments the one or more pesticide(s) include myclobutanil, pyrimethanil, carbaryl, permethrin, diazinon and ethoprophos.

In some embodiments the reduction in one or more heavy metal(s) is about 50%-100% by mass. In some embodiments the reduction in one or more heavy metal(s) is about 60%-100% by mass. In some embodiments the reduction in one or more heavy metal(s) is about 70%-100% by mass. In some embodiments the reduction in one or more heavy metal(s) is about 80%-100% by mass. In some embodiments the reduction in one or more heavy metal(s) is about 90%-100% by mass. In some embodiments the reduction in one or more heavy metal(s) is about 95%-100% by mass. In some embodiments the reduction in one or more heavy metal(s) is about 98%-100% by mass.

In some embodiments the reduction in arsenic is about 50%-100% by mass. In some embodiments the reduction in arsenic is about 60%-100% by mass. In some embodiments the reduction in arsenic is about 70%-100% by mass. In some embodiments the reduction in arsenic is about 80%-100% by mass. In some embodiments the reduction in arsenic is about 90%-100% by mass.

In some embodiments the reduction in lead is about 50%-100% by mass. In some embodiments the reduction in lead is about 60%-100% by mass. In some embodiments the reduction in lead is about 70%-100% by mass. In some embodiments the reduction in lead is about 80%-100% by mass. In some embodiments the reduction in lead is about 90%-100% by mass.

In some embodiments the reduction in one or more pesticide(s) is about 30%-100% by mass. In some embodiments the reduction in one or more pesticide(s) is about 40%-100% by mass. In some embodiments the reduction in one or more pesticide(s) is about 50%-100% by mass. In some embodiments the reduction in one or more pesticide(s) is about 60%-100% by mass.

In some embodiments the enriched cannabinoid extract has a reduced proportion of lipid compared to the crude Cannabis extract.

In some embodiments the reduction in lipid is about 50%-100% by mass. In some embodiments the reduction in lipid is about 60%-100% by mass. In some embodiments the reduction in lipid is about 70%-100% by mass. In some embodiments the reduction in lipid is about 80%-100% by mass. In some embodiments the reduction in lipid is about 90%-100% by mass. In some embodiments the reduction in lipid is about 95%-100% by mass. In some embodiments the reduction in lipid is about 98%-100% by mass. In some embodiments the reduction in lipid is about 99%-100% by mass. In some embodiments the reduction in lipid is about 99.5%-100% by mass.

In some embodiments the enriched cannabinoid extract comprises less than 10% lipid. In some embodiments the enriched cannabinoid extract comprises less than 5% lipid. In some embodiments the enriched cannabinoid extract comprises less than 1% lipid. In some embodiments the enriched cannabinoid extract comprises less than 0.05% lipid. In some embodiments the enriched cannabinoid extract comprises less than 0.05% lipid. In some embodiments the enriched cannabinoid extract comprises less than 0.01% lipid. In some embodiments the enriched cannabinoid extract comprises substantially no lipid.

In some embodiments the reduction in fat or wax is about 50%-100% by mass. In some embodiments the reduction in fat or wax is about 60%-100% by mass. In some embodiments the reduction in fat or wax is about 70%-100% by mass. In some embodiments the reduction in fat or wax is about 80%-100% by mass. In some embodiments the reduction in fat or wax is about 90%-100% by mass. In some embodiments the reduction in fat or wax is about 95%-100% by mass. In some embodiments the reduction in fat or wax is about 98%-100% by mass. In some embodiments the reduction in fat or wax is about 99%-100% by mass. In some embodiments the reduction in fat or wax is about 99.5%-100% by mass.

In some embodiments the enriched cannabinoid extract comprises less than 10% fat or wax. In some embodiments the enriched cannabinoid extract comprises less than 5% fat or wax. In some embodiments the enriched cannabinoid extract comprises less than 1% fat or wax. In some embodiments the enriched cannabinoid extract comprises less than 0.05% fat or wax. In some embodiments the enriched cannabinoid extract comprises less than 0.05% fat or wax. In some embodiments the enriched cannabinoid extract comprises less than 0.01% fat or wax. In some embodiments the enriched cannabinoid extract comprises substantially no fat or wax.

In some embodiments the enriched cannabinoid extract has an increased proportion of molecules within the size range 100 to 450 grams per mol, preferably more than 70%, more preferably greater than 75% and even more preferably greater than 80%.

In some embodiments the enriched cannabinoid extract has a reduced proportion of non-cannabinoid molecules that cause a bitter taste.

In some embodiments of the method of the fourth, fifth, sixth or seventh aspects the proportion of at least one terpene is increased.

In some embodiments of the method of the fourth, fifth, sixth or seventh aspects the total proportion of terpenes is increased.

In some embodiments of the method of the fourth, fifth, sixth or seventh aspects the proportion of at least one terpene is decreased.

In some embodiments of the method of the fourth, fifth, sixth or seventh aspects the total proportion of terpenes is decreased.

In some embodiments of the method of the fourth, fifth, sixth or seventh aspects the proportion of at least one terpene remains approximately the same.

In some embodiments of the method of the fourth, fifth, sixth or seventh aspects the total proportion of terpenes remains approximately the same.

In some embodiments the terpene is selected from one or more of linalool, caryophyllene oxide, guaiol, alpha-bisabolol, beta-caryophyllene, beta-myrcene, D-limonene, alpha-humulene, trans-nerolidol, geraniol, valencene, terpineol, borneol, camphene, delta-3-carene, eucalyptol, alpha-pinene, beta-pinene.

In some embodiments the terpene that increases is selected from one or more of linalool, caryophyllene oxide, guaiol, alpha-bisabolol, beta-caryophyllene, alpha-humulene, trans-nerolidol.

In some embodiments the proportion of linalool, caryophyllene oxide, guaiol, alpha-bisabolol, beta-caryophyllene, alpha-humulene and trans-nerolidol increase.

In some embodiments the terpene that decreases is selected from one or more of beta-myrcene, linalool, guaiol, beta-caryophyllene, D-limonene, alpha-humulene.

In some embodiments the proportion of beta-myrcene, linalool, guaiol, beta-caryophyllene, D-limonene and alpha-humulene decreases.

In some embodiments the terpene that remains approximately the same is selected from one or more of caryophyllene oxide, alpha-bisabolol, trans-nerolidol.

In some embodiments the proportion of caryophyllene oxide, alpha-bisabolol and trans-nerolidol remains approximately the same.

In some embodiments of the fourth, fifth, sixth or seventh aspects step (a) is followed by collection of the crude Cannabis extract that has been contacted with the imprinted polymer to give a Cannabis extract. In some embodiments the Cannabis extract is used in step (a) in place of the crude Cannabis extract. For example the crude Cannabis extract is contacted with the imprinted polymer more than once, by being collected and recontacting one or more times.

In some embodiments of the method of the fourth, fifth, sixth or seventh aspects the method further comprises the step prior to step (a) of forming an emulsion and/or dissolving the crude Cannabis extract in one or more liquid(s) to give an emulsion and/or solution of crude Cannabis extract, such that the imprinted polymer is contacted with the crude Cannabis extract in the form of an emulsion and/or solution.

In some embodiments the emulsion and/or solution is substantially uniformly dispersed.

In some embodiments the emulsion and/or solution is prepared by sonication/ultrasonication and/or high shear mixing of the crude Cannabis extract and the one or more liquid(s).

In some embodiments the liquid(s) comprise one or more of water, ethanol, methanol, ethyl acetate, isopropyl alcohol, acetonitrile, acetone or THF.

In some embodiments the liquid(s) are selected from ethanol or water or a mixture thereof.

In some embodiments the crude extract is dissolved/emulsified in about 5 to 100 ml of the one or more liquids per gram of the crude extract. In some embodiments the crude extract is dissolved/emulsified in about 5 to 80 ml of the one or more liquids per gram of the crude extract. In some embodiments the crude extract is dissolved/emulsified in about 5 to 60 ml of the one or more liquids per gram of the crude extract. In some embodiments the crude extract is dissolved/emulsified in about 5 to 50 ml of the one or more liquids per gram of the crude extract.

In some embodiments of the fourth, fifth, sixth or seventh aspects the method further comprises a rinse step following step (a) and prior to step (b) of washing the imprinted polymer with a rinse liquid to remove at least a portion of undesired components from the crude extract.

In some embodiments, where steps (a) and (b) are repeated, the rinse step is optionally repeated one or more times.

In some embodiments the rinse liquid comprises one or more of water, ethanol, methanol, ethyl acetate, isopropyl alcohol, acetonitrile, acetone, tetrahydrofuran (THF).

In some embodiments of the fourth, fifth, sixth or seventh aspects step (b) is followed by collection of the enriched Cannabis extract that has been produced by eluting the cannabinoids from the imprinted polymer. In some embodiments the enriched Cannabis extract is used in step (a) in place of the crude Cannabis extract.

In some embodiments the enriched cannabinoid extract from step (b) used in place of the crude Cannabis extract in step (a) and step (a) and step (b) are repeated using the enriched cannabinoid extract.

In some embodiments the eluent is reduced to a desired volume or increased to a desired volume prior to repeating step (b).

In some embodiments of the fourth, fifth, sixth or seventh aspects the elution solvent comprises one or more of ethanol, methanol, ethyl acetate, isopropyl alcohol, acetonitrile, acetone or THF.

In some embodiments the elution solvent is mixture of more than one solvent.

In some embodiments the proportions of the solvents change over the course of step (b).

In some embodiments the elution solvent comprises ethanol.

In some embodiments the elution solvent consists of ethanol.

In some embodiments the step of eluting the cannabinoids from the imprinted polymer with an elution solvent comprises collecting the eluent in one or more than one portion.

In some embodiments of the fourth, fifth, sixth or seventh aspects the method further comprises the step following step (b) of regenerating the imprinted polymer using a regeneration solvent.

In some embodiments the regeneration solvent comprises one or more of isopropyl alcohol, acetone, an alkane (for example hexane).

In some embodiments the crude Cannabis extract is produced by extraction of plant matter with a solvent.

In some embodiments the crude Cannabis extract is produced by extraction with supercritical carbon dioxide, subcritical carbon dioxide, ethanol, one or more hydrocarbons (for example propane, butane, hexane).

In some embodiments the crude Cannabis extract comprises a substantial proportion of at least one non-cannabinoid material selected from one or more of lipids (including waxes, fats, wax esters), plant pigments, glycerides, unsaturated fatty acids, one or more pesticide contaminants, one or more heavy metal contaminants, terpenes, carotenes, flavonoids.

In some embodiments the one or more heavy metal(s) include arsenic, cadmium, chromium, copper, lead, nickel and zinc.

In some embodiments the one or more pesticide(s) include myclobutanil, pyrimethanil, carbaryl, permethrin, diazinon and ethoprophos.

In some embodiments the crude Cannabis extract comprises at least 1% lipid. In some embodiments the crude Cannabis extract comprises at least 2% lipid. In some embodiments the crude Cannabis extract comprises at least 3% lipid. In some embodiments the crude Cannabis extract comprises at least 4% lipid. In some embodiments the crude Cannabis extract comprises at least 5% lipid.

In some embodiments the crude Cannabis extract comprises about 1% to 60% lipid. In some embodiments the crude Cannabis extract comprises about 2% to 60% lipid. In some embodiments the crude Cannabis extract comprises about 3% to 60% lipid. In some embodiments the crude Cannabis extract comprises about 4% to 60% lipid. In some embodiments the crude Cannabis extract comprises about 5% to 60% lipid.

In some embodiments the crude Cannabis extract comprises about 1% to 50% lipid. In some embodiments the crude Cannabis extract comprises about 2% to 50% lipid. In some embodiments the crude Cannabis extract comprises about 3% to 50% lipid. In some embodiments the crude Cannabis extract comprises about 4% to 50% lipid. In some embodiments the crude Cannabis extract comprises about 5% to 50% lipid.

In some embodiments the crude Cannabis extract comprises at least 1% fat or wax. In some embodiments the crude Cannabis extract comprises at least 2% fat or wax. In some embodiments the crude Cannabis extract comprises at least 3% fat or wax. In some embodiments the crude Cannabis extract comprises at least 4% fat or wax. In some embodiments the crude Cannabis extract comprises at least 5% fat or wax.

In some embodiments the crude Cannabis extract comprises about 1% to 60% fat or wax. In some embodiments the crude Cannabis extract comprises about 2% to 60% fat or wax. In some embodiments the crude Cannabis extract comprises about 3% to 60% fat or wax. In some embodiments the crude Cannabis extract comprises about 4% to 60% fat or wax. In some embodiments the crude Cannabis extract comprises about 5% to 60% fat or wax.

In some embodiments the crude Cannabis extract comprises about 1% to 50% fat or wax. In some embodiments the crude Cannabis extract comprises about 2% to 50% fat or wax. In some embodiments the crude Cannabis extract comprises about 3% to 50% fat or wax. In some embodiments the crude Cannabis extract comprises about 4% to 50% fat or wax. In some embodiments the crude Cannabis extract comprises about 5% to 50% fat or wax.

In some embodiments the crude Cannabis extract is not winterized.

In an eighth aspect there is provided an enriched cannabinoid extract comprising:
greater than 65% combined mass of one or more cannabinoid(s),
at least one cannabinoid selected from the group CBD, THC, CBN, CBND, CBC, THCV, CBL, CBE and CBDV;
greater than about 0.03% by weight CBG; and
at least one terpene, selected from linalool, caryophyllene oxide, guaiol, alpha-bisabolol, beta-caryophyllene, alpha-humulene, trans-nerolidol.

In some embodiments the enriched cannabinoid extract comprises less than 10% lipid. In some embodiments the enriched cannabinoid extract comprises less than 5% lipid. In some embodiments the enriched cannabinoid extract comprises less than 1% lipid. In some embodiments the enriched cannabinoid extract comprises less than 0.05% lipid. In some embodiments the enriched cannabinoid extract comprises less than 0.05% lipid. In some embodiments the enriched cannabinoid extract comprises less than 0.01% lipid. In some embodiments the enriched cannabinoid extract comprises substantially no lipid.

In some embodiments the enriched cannabinoid extract comprises about 0-10% lipid. In some embodiments the enriched cannabinoid extract comprises about 0-5% lipid. In some embodiments the enriched cannabinoid extract comprises about 0-1% lipid. In some embodiments the enriched cannabinoid extract comprises about 0-0.05% lipid. In some embodiments the enriched cannabinoid extract comprises about 0-0.05% lipid. In some embodiments the enriched cannabinoid extract comprises about 0-0.01% lipid.

In some embodiments the enriched cannabinoid extract comprises less than 10% fat or wax. In some embodiments the enriched cannabinoid extract comprises less than 5% fat or wax. In some embodiments the enriched cannabinoid extract comprises less than 1% fat or wax. In some embodiments the enriched cannabinoid extract comprises less than 0.05% fat or wax. In some embodiments the enriched cannabinoid extract comprises less than 0.05% fat or wax. In some embodiments the enriched cannabinoid extract comprises less than 0.01% fat or wax. In some embodiments the enriched cannabinoid extract comprises substantially no fat or wax.

In some embodiments the enriched cannabinoid extract comprises less than 0.10% by mass of a pesticide residue. In some embodiments the enriched cannabinoid extract comprises less than 0.05% by mass of a pesticide residue. In some embodiments the enriched cannabinoid extract comprises less than 0.01% by mass of a pesticide residue. In some embodiments the enriched cannabinoid extract is substantially free from a pesticide residue.

In some embodiments the enriched cannabinoid extract comprises less than 0.1% by mass total pesticide residue. In some embodiments the enriched cannabinoid extract comprises less than 0.05% by mass total pesticide residue. In some embodiments the enriched cannabinoid extract comprises less than 0.01% by mass total pesticide residue. In some embodiments the enriched cannabinoid extract is substantially free from pesticide residue.

In some embodiments the pesticide includes myclobutanil, pyrimethanil, carbaryl, permethrin, diazinon and/or ethoprophos.

In some embodiments the enriched cannabinoid extract comprises less than 0.1% by mass heavy metals. In some embodiments the enriched cannabinoid extract comprises less than 0.05% by mass heavy metals. In some embodiments the enriched cannabinoid extract comprises less than 0.01% by mass heavy metals. In some embodiments the enriched cannabinoid extract comprises less than 0.001% by mass heavy metals. In some embodiments the enriched cannabinoid extract is substantially free from heavy metals. In some embodiments the heavy metals include arsenic, cadmium, chromium, copper, lead, nickel and/or zinc.

In some embodiments the enriched cannabinoid extract comprises less than about 2% by mass non-cannabinoid molecules that cause bitter taste. In some embodiments the enriched cannabinoid extract comprises less than about 1% by mass non-cannabinoid molecules that cause bitter taste. In some embodiments the enriched cannabinoid extract comprises less than about 0.5% by mass non-cannabinoid molecules that cause bitter taste. In some embodiments the enriched cannabinoid extract comprises less than about 0.1% by mass non-cannabinoid molecules that cause bitter taste. In some embodiments the enriched cannabinoid extract comprises less than about 0.1% by mass non-cannabinoid molecules that cause bitter taste. In some embodiments the enriched cannabinoid extract is substantially free from non-cannabinoid molecules that cause bitter taste.

In some embodiments the enriched cannabinoid extract comprises less than 30%, preferably less than 25%, and more preferably less than 15% content by weight of molecules found within Cannabis plant material that fall outside the size range of about 100 to 450 grams per mol.

In some embodiments the enriched cannabinoid extract comprises greater than 1.15% by weight CBG. In some embodiments the enriched cannabinoid extract comprises greater than 2% by weight CBG. In some embodiments the enriched cannabinoid extract comprises between about 2-10% by weight CBG. In some embodiments the enriched cannabinoid extract comprises between about 2-5% by weight CBG.

In some embodiments the enriched cannabinoid extract comprises greater than about 70% combined mass of one or more cannabinoid(s). In some embodiments the enriched cannabinoid extract comprises greater than about 75% combined mass of one or more cannabinoid(s). In some embodiments the enriched cannabinoid extract comprises greater than about 80% combined mass of one or more cannabinoid(s).

In some embodiments the enriched cannabinoid extract comprises at least one cannabinoid acid selected from the group CBDA, THCA, CBGA, CBCA, CBLA, CBEA-A and CBEA-B. In some embodiments the enriched cannabinoid extract comprises at least two cannabinoid acids selected from the group CBDA, THCA, CBGA, CBCA, CBLA, CBEA-A and CBEA-B.

In some embodiments the enriched cannabinoid extract comprises CBDA.

In some embodiments the enriched cannabinoid extract comprises more than one terpene.

In some embodiments the enriched cannabinoid extract comprises linalool. In some embodiments the enriched cannabinoid extract comprises caryophyllene oxide. In some embodiments the enriched cannabinoid extract comprises guaiol. In some embodiments the enriched cannabinoid extract comprises alpha-bisabolol. In some embodiments the enriched cannabinoid extract comprises beta-caryophyllene. In some embodiments the enriched cannabinoid extract comprises alpha-humulene. In some embodiments the enriched cannabinoid extract comprises trans-nerolidol.

Any of the aforementioned features or embodiments or aspects may be combined with one or more of the other features or embodiments or aspects as described herein.

The term “enriched cannabinoid extract” encompasses preparations that have an increased total or an increase in one or more cannabinoid(s) over crude Cannabis extract, preferably having at least about 65%, preferably at least about 70%, preferably at least about 80%, preferable at least about 85%, more preferably at least about 90% chromatographic purity for the desired cannabinoid or cannabinoid acid or total cannabinoid content. The enriched cannabinoid extract may include other components such as terpenes/terpenoids.

The term “comprising” as used in this specification and claims means “consisting at least in part of”. When interpreting each statement in this specification and claims that includes the term “comprising”, features other than that or those prefaced by the term may also be present. Related terms such as “comprise” and “comprises” are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

As used herein the term “and/or” means “and” or “or”, or both.

As used herein “(s)” following a noun means the plural and/or singular forms of the noun.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

The disclosure consists in the foregoing and also envisages constructions of which the following gives examples only. Features disclosed herein may be combined into new embodiments of compatible components addressing the same or related inventive concepts.

### BRIEF DESCRIPTION OF THE FIGURES

Preferred embodiments of the disclosure will be described by way of example only and with reference to the following drawings.

FIG. 1 shows total cannabinoids in the enriched extract after being passed through EGDMA/TBA imprinted beads.

FIG. 2 shows total cannabinoids in each fraction of the enriched extract after being passed through EGDMA/TBA imprinted beads.

FIGS. 3A, 3B, 3C, 3D and 3E show changes in the cannabinoid profile in different eluent fractions after being passed through EGDMA/TBA imprinted beads.

FIG. 4 shows cannabinoid percentages over 5 cycles.

FIG. 5 shows the filter paper used to filter the crude extract filter after winterization.

FIG. 6 shows the filter paper used to filter the eluent after exposure to the MIP and after winterization.

FIG. 7 shows the filter paper used to filter the crude extract after winterization.

FIG. 8 shows the filter paper used to filter the eluent after exposure to the MIP and after winterization.

### DETAILED DESCRIPTION

The present disclosure relates to an imprinted polymer and/or an imprinted polymer bead for producing an enriched cannabinoid extract from a crude Cannabis extract. The disclosure further or alternatively relates to a method of making an imprinted polymer for producing an enriched cannabinoid extract from a crude Cannabis extract and/or a method of producing an enriched cannabinoid extract from a crude Cannabis extract and/or a method of reducing heavy metal content in an enriched cannabinoid extract when compared to a crude Cannabis extract and/or a method of reducing at least one pesticide residue in an enriched cannabinoid extract when compared to a crude Cannabis extract and/or a method of reducing lipid extraction steps when producing an enriched cannabinoid extract from a crude Cannabis extract and/or a method of producing an enriched cannabinoid extract from a crude non-winterized Cannabis extract and/or an enriched cannabinoid extract.

Enriched cannabinoid extracts are in increasing demand, but producing them requires multiple steps which are complex and can be expensive. The product must also be safe to be consumed, so must remove toxic components from crude Cannabis extracts and must not introduce toxic components into the enriched extracts.

The present invention provides an imprinted polymer for producing an enriched cannabinoid extract from a crude Cannabis extract, wherein the polymer is imprinted with a template organic molecule with a molecular weight of about 150 to 450 grams per mol and comprising a hydroxyphenyl group, and wherein the polymer has been prepared from one or more polymerizable monomers.

Alternatively the present invention provides an imprinted polymer bead for producing an enriched cannabinoid extract from a crude Cannabis extract, wherein the imprinted polymer bead is imprinted with a template organic molecule with a molecular weight of about 150 to 450 grams per mol comprising a hydroxyphenyl group, and wherein the imprinted polymer bead has been prepared from one or more polymerizable monomer(s).

Alternatively the present invention provides a method of making an imprinted polymer for producing an enriched cannabinoid extract from a crude Cannabis extract, the method comprising polymerizing one or more polymerizable monomers in the presence of a template organic molecule with a molecular weight of about 150 to 450 grams per mol and comprising a hydroxyphenyl group, and subsequently at least partially removing the template molecule from the imprinted polymer.

Alternatively the present invention provides a method of producing an enriched cannabinoid extract from a crude Cannabis extract, the method comprising the steps of: a) contacting an imprinted polymer with the crude Cannabis extract, wherein the imprinted polymer is a polymer that has been imprinted with a template organic molecule with a molecular weight of about 150 to 450 grams per mol and comprised a hydroxyphenyl group, and wherein the polymer has been prepared from one or more polymerizable monomers selected; and b) eluting the cannabinoids from the imprinted polymer with an elution solvent to produce the enriched cannabinoid extract comprising one or more cannabinoid(s).

Alternatively the present invention provides a method of reducing heavy metal content in an enriched cannabinoid extract when compared to a crude Cannabis extract, the method comprising the steps of: a) contacting an imprinted polymer with the crude Cannabis extract, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers; b) eluting the cannabinoids from the imprinted polymer with an elution solvent to produce the enriched cannabinoid extract comprising one or more cannabinoid(s).

Alternatively the present invention provides a method of reducing at least one pesticide residue in an enriched cannabinoid extract when compared to a crude Cannabis extract, the method comprising the steps of: a) contacting an imprinted polymer with the crude Cannabis extract, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers; b) eluting the cannabinoids from the imprinted polymer with an elution solvent to produce the enriched cannabinoid extract comprising one or more cannabinoid(s).

Alternatively the present invention provides a method of reducing lipid extraction steps when producing an enriched cannabinoid extract from a crude Cannabis extract, the method comprising the steps of: a) contacting an imprinted polymer with the crude Cannabis extract containing lipid, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers; b) eluting the cannabinoids from the imprinted polymer with an elution solvent to produce the enriched cannabinoid extract comprising one or more cannabinoid(s).

Alternatively the present invention provides an enriched cannabinoid extract comprising greater than 65% combined mass of one or more cannabinoid(s), at least one cannabinoid selected from the group CBD, THC, CBN, CBND, CBC, THCV, CBL, CBE and CBDV; greater than about 0.03% by weight CBG; and at least one terpene, selected from linalool, caryophyllene oxide, guaiol, alpha-bisabolol, beta-caryophyllene, alpha-humulene, trans-nerolidol.

### Monomer(s)

Molecularly-imprinted polymers (MIP or “imprinted polymer”) are polymers with an antibody-like ability to bind and discriminate between molecules. Molecularly-imprinted polymers are formed by the synthesis of cross-linked polymers in the presence of a template small molecule. The template molecule is then removed leaving behind a structure in the polymer complementary to the template molecule, for example a “pocket” or other complimentary binding area. The imprinted polymer may bind the small molecule of interest (e.g. cannabinoids) in the pocket or binding area covalently or non-covalently, for example, with hydrogen bonding, other electrostatic interactions, aromatic stacking or hydrophobic effects.

Described herein is an imprinted polymer and/or imprinted polymer bead and/or a method of making an imprinted polymer and/or a method of producing an enriched cannabinoid extract from a crude Cannabis extract, method of reducing heavy metal content in an enriched cannabinoid extract when compared to a crude Cannabis extract, a method of reducing at least one pesticide residue in an enriched cannabinoid extract, a method of reducing lipid extraction steps when producing an enriched cannabinoid extract from a crude Cannabis extract and/or a a method of producing an enriched cannabinoid extract from a crude non-winterized Cannabis extract, that makes use of an imprinted polymer.

The imprinted polymers are prepared from one or more polymerizable monomers, preferably selected from an acryl or a vinyl monomer(s).

The acryl monomer is preferably selected from acrylic acid, tert-butylacrylamide, N-phenylacrylamide, N-methylacrylamide, methacrylic acid, 2-(hydroxyethyl)methacrylate, ethylene glycol dimethacrylate, methacrylic anhydride or trimethylolpropane trimethacrylate. The acryl monomer may be a methacryl monomer. The methacryl monomer is preferably selected from methacrylic acid, 2-(hydroxyethyl)methacrylate, ethylene glycol dimethacrylate, methacrylic anhydride or trimethylolpropane trimethacrylate.

The vinyl monomer is preferably selected from a styryl or vinylpyridine. The styryl monomer is preferably selected from styrene or 4-vinylstytrene. The vinylpyridine monomer is preferably 4-vinylpyridine

Preferably the polymer is prepared from one or more acryls (including methacryl) monomers or one or more styryl monomers. The polymer is preferably prepared from two or more monomers, for example, two or more acryl (including methacryl) monomers, or two or more styryl monomers, or a mixture of two of more of a acryl or a styryl. Preferably at least one of the monomers in the matrix of the polymer acts as a crosslinker. The crosslinker usually forms the bulk of the polymer. In some cases a crosslinking monomer can be used as the only monomer. The crosslinker has at least two functional groups that can polymerize. Preferably the crosslinker is a diolefin. Examples of crosslinkers are divinyl benzene and ethylene glycol dimethacrylate (EGDMA). Preferably the polymer has been prepared from divinylbenzene (DVB) and styrene monomers. Alternatively, preferably the polymer has been prepared from tert-butylacryamide (TBA) and ethylene glycol dimethacrylate (EGDMA) monomers. Alternatively, preferably the polymer has been prepared from a single crosslinking monomer, and preferably without a non-crosslinking monomer, preferably the single crosslinking monomer is ethylene glycol dimethacrylate (EGDMA) monomer.

Where the polymer matrix is prepared from divinylbenzene (DVB) and styrene the ratio of divinylbenzene (DVB) and styrene is preferably about 10-6:1, i.e. between 10 and 6 parts DVB to 1 part styrene by mass.

Where the polymer matrix is prepared from ethylene glycol dimethacrylate (EGDMA) and tert-butylacryamide (TBA) monomers the ratio of ethylene glycol dimethacrylate (EGDMA) and tert-butylacryamide (TBA) is preferably about 1:0.05-0.5 by molar mass, or about 1:0.05-0.3 by molar mass, or about 1:0.1-0.2 by molar mass.

Where the polymer matrix is prepared from divinylbenzene (DVB) and styrene monomers the ratio of divinylbenzene (DVB) and styrene is preferably about 10-26:1:0.01-0.5 by molar mass, preferably about 1:0.1-0.3 by molar mass.

Where the polymer matrix is prepared from ethylene glycol dimethacrylate (EGDMA) and methacrylic acid, the ratio of ratio of ethylene glycol dimethacrylate (EGDMA) and methacrylic acid is preferably about 1:0.1-2 by molar mass, preferably about 1:0.2-0.8 by molar mass, preferably about 1:0.4-0.5 by molar mass.

Where the polymer matrix is prepared from acrylic acid and ethylene glycol dimethacrylate (EGDMA), the ratio of acrylic acid and ethylene glycol dimethacrylate (EGDMA) is preferably about 1:0.2-1.1 by molar mass, preferably about 1:0.4-0.8 by molar mass, preferably about 1:0.6-0.7 by molar mass.

Where the polymer matrix is prepared from ethylene glycol dimethacrylate (EGDMA) and 2-(hydroxyethyl) methacrylate, the ratio of ethylene glycol dimethacrylate (EGDMA) and 2-(hydroxyethyl) methacrylate is preferably about 1:1.5-0.1 by molar mass, preferably about 1:1.2-0.8 by molar mass, preferably about 1:0.5-0.1 by molar mass.

Wherein the polymer matrix is prepared from styrene and ethylene glycol dimethacrylate (EGDMA) monomers, the ratio of ethylene glycol dimethacrylate (EGDMA) and styrene is preferably about 1:0.1-1 5-1:1 by molar mass, preferably about 1:0.3-0.9 by molar mass, preferably about 1:0.6-0.7 by molar mass.

An advantage of preparing the polymer matrix from a single crosslinking monomer (for example ethylene glycol dimethacrylate (EGDMA) as the only monomer), is that there is no need to measure or monitor the ratio of monomers (because there is only one). This allows for less process steps, which is an advantage particularly in industrial manufacturing.

### Beads

In a preferred embodiment the imprinted polymer is in the form of a bead. This allows for a greater surface area of the imprinted polymer to be exposed to the crude Cannabis extract, than for example a sheet of polymer. Beads are also convenient for processing, for example than a powder, as they allow the input matrix material (emulsion and/solution of the crude product) and the elution solvent to pass over/through the beads at a reasonable rate for commercial processing.

The bead is preferably about 0.1 to 10 mm in diameter, about 0.2 to 8 mm in diameter, about 0.2 to 6 mm in diameter, about 0.2 to 5 mm in diameter, about 0.5 to 6 mm in diameter, about 0.5 to 4 mm in diameter, about 0.5 to 3 mm in diameter, about 0.5 to 2 mm in diameter, about 0.5-1.5 mm diameter or about 1 to 4 mm in diameter.

Preferably about 99% of the beads are about 0.1 to 10 mm in diameter, about 95% of the beads are about 0.1 to 10 mm in diameter, about 90% of the beads are about 0.1 to 10 mm in diameter, about 85% of the beads are about 0.1 to 10 mm in diameter.

The bead preferably has compression strength of about 300-13,800 psi.

### Template

The imprinted polymer is formed in the presence of a template molecule which is removed after the polymer is formed leaving a “pocket” or other complimentary binding area structure that may bind a target molecule (for example the molecule(s) being enriched). The template does not act as a monomer (i.e. the template is not covalently bonded to the monomers used to prepare the polymer) so that the template may be removed from the imprinted polymer using a solvent. While not wishing to be bound by theory, it is believed there is likely to be other forms of weaker bonding between the template and the monomer, for example hydrogen bonding, aromatic stacking interactions, and/or Van der Waals forces.

The template molecule used in the present invention preferably has a molecular weight of about 150 to 450 grams per mol (not including any associated salts or water molecules of hydration).

For use in the present invention the template preferably comprises the structure:

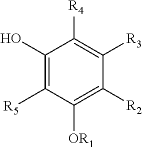

wherein R₁, R₂, R₃, R₄ and R₅ are the remainder of the organic molecule.

Preferably the template preferably comprises the structure:

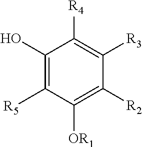

wherein R₁, R₂, R₃, R₄ and R₅ are the remainder of the organic molecule wherein
R₁ is H
R₂ is selected from H, or an organic group,
R₃ is selected from H, —OH, or an organic group,
R₄ is H or an organic group,
R₅ is H, or an organic group,
or R₁ and R₂ together form a 5- or 6-membered ring, preferably a 6-membered ring, optionally substituted with one or more of —OH, or an organic group, or
or R₁ and R₅ together form a 5- or 6-membered ring, preferably a 6-membered ring,
optionally substituted with one or more substitutes independently selected from —OH, or an organic group, and wherein the 5- or 6-membered ring is optionally fused to a further ring which may be optionally substituted with one or more alkyl.

The term “organic group” as used herein means a group comprising one or more carbon atoms and optionally one or more hydrogen, halogen, nitrogen, sulfur, and oxygen atoms, obtainable by removing one hydrogen atom at the point of attachment to the parent compound.

The template preferably consists of C, H and O atoms, i.e. there are no other types of atom, for example nitrogen or phosphorous, that may be found in organic molecules.

One or more of the R₁, R₂, R₃, R₄ or R₅ groups is preferably a saturated alkyl group, preferably a saturated alkyl group with 2 to 8 carbons.

Preferably at least one of R₂, R₃, and R₅ are an alkyl, preferably a 4-6 alkyl, or R₁ and R₂ or R₁ and R₅ together form the 5- or 6-membered ring.

R₃ is preferably a C2-C8 saturated alkyl group, which may be branched or unbranched. R₃ is preferably an unbranched pentyl group.

Preferably R₂ and R₃ or R₃ and R₄ or R₁ and R₂ or R₁ and R₅ form a fused ring, preferably a 5- or 6-membered ring, preferably a 6-membered ring wherein the fused ring is optionally substituted. In some embodiments the fused ring is substituted with a substituted or unsubstituted phenyl group. In some embodiments the phenyl group is substituted with one or more alcohol groups. In some embodiments the fused ring is substituted with an alcohol group.

In some embodiments:
R₁ is H
R₂ is selected from H, alkyl, or CORₓ, wherein Rₓ is alkyl, cycloalkyl or aryl, preferably aryl,
R₃ is selected from H, —OH or alkyl,
R₄ is H or alkyl,
R₅ is H, alkyl, or cycloalkyl optionally substituted with one or more alkyl or —OH,
or R₁ and R₂ together form a 5- or 6-membered ring, preferably a 6-membered ring,
optionally substituted with one or more substituents independently selected from —OH, alkyl, or aryl, wherein the aryl is optionally substituted with one or more —OH,
or R₁ and R₅ together form a 5- or 6-membered ring, preferably a 6-membered ring,
optionally substituted with one or more substitutents independently selected from —OH, alkyl, or aryl, wherein the aryl is optionally substituted with one or more —OH, and wherein the 5- or 6-membered ring is optionally fused to a further 5- or 6-membered ring, preferably a cycloalkyl ring, which may be optionally substituted with one or more independently selected alkyl.

Preferably wherein R₂ is alkyl; and R₁, R₃, R₄, and R₅ are H.

Preferably wherein R₃ is alkyl; and R₁, R₂, R₄, and R₅ are H.

Preferably wherein R₂ is CORₓ wherein Rₓ is aryl, and R₁, R₃, R₄, and R₅ are H.

Preferably wherein R₃ and R₅ are each independently alkyl; and R₁, R₂, and R₄ are H.

Preferably wherein R₃ is alkyl; R₅ is cycloalkyl optionally substituted with one or more alkyl groups, and R₁, R₂, and R₄ are H.

Preferably wherein R₃ is OH; R₄ and R₅ are H; and R₁ and R₂ together form a 6-membered ring optionally substituted with one or more groups independently selected from —OH and phenyl, wherein the phenyl is optionally substituted with one or more —OH.

Preferably wherein R₃ is alkyl; R₂ and R₄ are H; and R₁ and R₅ together form a 6-membered ring optionally substituted with one or more substituents independently selected from —OH, and alkyl, and wherein the 6-membered ring is optionally fused to a further 6-membered ring which may be optionally substituted with one or more independently selected alkyl.

In some embodiments, the template is of the formula:

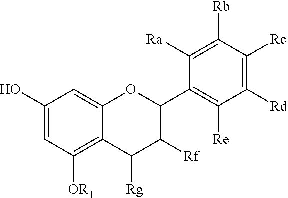

wherein Rₐ, R_{b}, R_{c}, R_{d}, Rₑ, R_{f} and R_{g} are optional substituents. Preferably Rₐ, R_{b}, R_{c}, R_{d}, Rₑ are independently —OH or —H. Preferably, R_{b}, R_{c} are —OH groups. Preferably R_{b}, R_{c} are —OH groups and Rₐ, R_{d} and Rₑ are hydrogen groups.

The template can be one of the molecules of interest, i.e. the molecule that is intended to be enriched in the cannabinoid extract, for example a cannabinoid. Preferably the template is selected from Cannabigerol (CBG), Cannabidiol (CBD), Tetrahydrocannabinol (THC). For example, a cannabinoid template may be used with a polymer made from a EGDMA monomer, for example, either EGDMA monomer alone or EGDMA with methacrylic acid.

In some embodiments the template is selected from:

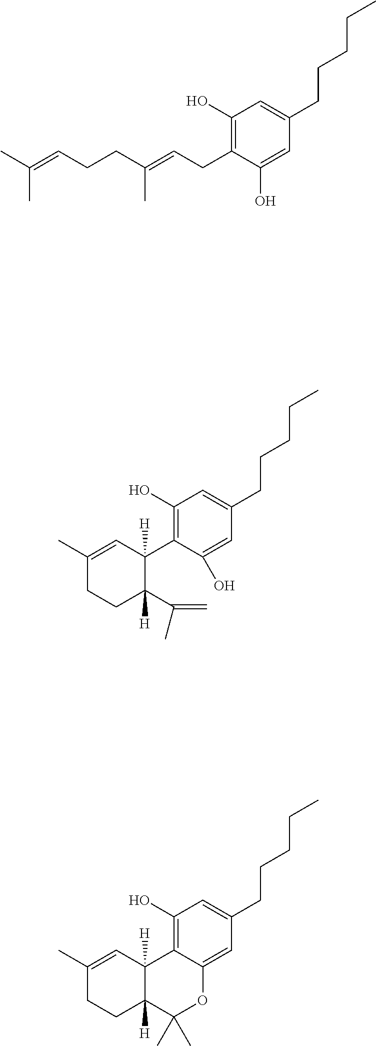

The inventors have found useful enriched cannabinoid extract can alternatively be obtained where the template is not a cannabinoids, for example the template is an alkyl- or acyl-resorcinol, preferably 2,4-Dihydroxybenzophenone, olivetol or 4-Hexylresorcinol, or a flavonoid or more preferably a flavan-3-ol. Preferably the template is selected from 2,4-Dihydroxybenzophenone, Olivetol, 4-Hexylresorcinol, Catechin. Preferably the template is (+/−)-catechin.

Preferably the ratio of the template to the one or more monomer(s) is about 1:10-300 by molar mass, about 1:15-200 by molar mass, about 1:20-160 by molar mass, about 1:50-150 by molar mass, or about 1:70-120 by molar mass.

The term “alkyl” employed alone or in combination with other terms, encompasses alkenyl, cycloalkyl, cycloalkenyl, and unless indicated otherwise, refers to a straight chain or branched chain hydrocarbon group having from 1 to 12 carbon atoms. In some embodiments, alkyl groups have from 1 to 10, from 1 to 8, from 1 to 6, or from 1 to 4 carbon atoms. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl. The term “alkenyl” employed alone or in combination with other terms, unless indicated otherwise, refers to a straight or branched chain hydrocarbon group having from 2 to 12 carbon atoms and having at least one double bond between two carbon atoms. In some embodiments, alkenyl groups have from 2 to 10, from 2 to 8, from 2 to 6, or from 2 to 4 carbon atoms. In some embodiments, alkenyl groups have one, two, or three carbon-carbon double bonds. Examples of alkenyl groups include, but are not limited to, vinyl, allyl, —CH═CH(CH3), —CH═C(CH3)2, —C(CH3)═CH2, and —C(CH3)=CH(CH3).

The term “cycloalkyl” employed alone or in combination with other terms, unless indicated otherwise, refers to a mono-, bi- or tricyclic hydrocarbon group having from 3 to 12 carbon atoms in the ring(s). In some embodiments, cycloalkyl groups have from 3 to 10, from 3 to 8, from 3 to 7, from 3 to 6, from 4 to 6, from 3 to 5 or from 4 to 5 carbon atoms in the ring(s). In some embodiments, cycloalkyl groups have 5 or 6 ring carbon atoms. Examples of monocyclic cycloalkyl groups include, but are not limited to, cyclopropyl, cyclobutyl, cyclopentyl, cyclohexyl, cycloheptyl, and cyclooctyl. Bi- and tricyclic ring systems include bridged, spiro, and fused cycloalkyl ring systems. Examples of bi- and tricyclic ring cycloalkyl systems include, but are not limited to, bicyclo[2.1.1]hexanyl, bicyclo[2.2.1]heptanyl, adamantyl, and decalinyl.

The term “cycloalkenyl” employed alone or in combination with other terms, unless indicated otherwise, refers to a non-aromatic mono-, bi- or tricyclic hydrocarbon groups having from 4 to 12 carbon atoms in the ring(s) and having at least one double bond between two carbon atoms. In some embodiments, cycloalkenyl groups have one, two or three double bonds. In some embodiments, cycloalkenyl groups have from 5 to 12, from 5 to 10, from 5 to 8, or from 5 to 6 carbon atoms in the ring(s). In some embodiments, cycloalkenyl groups have 5, 6, 7, or 8 ring carbon atoms in the ring(s). Examples of cycloalkenyl groups include cyclohexenyl, cyclopentenyl, cyclohexadienyl, butadienyl, pentadienyl, and hexadienyl.

The term “aryl” employed alone or in combination with other terms, unless indicated otherwise, refers to a cyclic aromatic hydrocarbon group having from 6 to 14 carbon atoms in the ring(s) and no heteroatoms in the ring(s). Aryl groups include monocyclic, fused bicyclic, and fused tricyclic ring systems. Examples of aryl groups include, but are not limited to, phenyl, azulenyl, heptalenyl, fluorenyl, phenanthrenyl, anthracenyl, indenyl, indanyl, pentalenyl, and naphthyl. In some embodiments, aryl groups have from 6 to 12, or from 6-10 carbon atoms in the ring(s). In some embodiments, the aryl groups are phenyl or naphthyl. Aryl groups include aromatic-aliphatic fused ring systems. Examples include, but are not limited to, indanyl and tetrahydronaphthyl.

The term “heterocyclyl” employed alone or in combination with other terms, unless indicated otherwise, refers to a non-aromatic ring system containing from 3 to 16 atoms in the ring(s), of which one or more is a heteroatom. In some embodiments, the heteroatom is nitrogen, oxygen, or sulfur. In some embodiments, the heterocyclyl group contains one, two, three, or four heteroatoms. In some embodiments, heterocyclyl groups include mono-, bi- and tricyclic rings having from 3 to 16, from 3 to 14, from 3 to 12, from 3 to 10, from 3 to 8, or from 3 to 6 atoms in the ring(s). Heterocyclyl groups include partially unsaturated and saturated ring systems, for example, imidazolinyl and imidazolidinyl. Heterocyclyl groups include fused and bridged ring systems containing a heteroatom, for example, quinuclidyl. Heterocyclyl groups include, but are not limited to, aziridinyl, azetidinyl, azepanyl, diazepanyl, 1,3-dioxanyl, 1,3-dioxolanyl, isoxazolidinyl, morpholinyl, piperazinyl, piperidinyl, pyranyl, pyrazolidinyl, pyrrolinyl, pyrrolidinyl, tetrahydrofuranyl, tetrahydrothienyl, thiadiazolidinyl, and trithianyl. In some embodiments, heterocyclyl groups have 5 or 6 ring carbon atoms.

The term “heteroaryl” employed alone or in combination with other terms, unless indicated otherwise, refers to an aromatic ring system containing from 5 to 16 atoms in the ring(s) and at least one heteroatom in the ring(s). In some embodiments, the heteroatom is nitrogen, oxygen, sulfur, or selenium, preferably oxygen, nitrogen, or sulfur. In some embodiments, heteroaryl groups comprise 1, 2, or 3 heteroatoms in the ring(s). In some embodiments, heteroaryl groups include monocyclic, fused bicyclic, and fused tricyclic ring systems having from 5 to 16, from 5 to 14, from 5 to 12, from 5 to 10, from 5 to 8, or from 5 to 6 atoms in the ring(s). Heteroaryl groups include, but are not limited to, pyrrolyl, pyrazolyl, triazolyl, tetrazolyl, oxazolyl, isoxazolyl, thiazolyl, pyridinyl, pyridazinyl, pyrimidinyl, pyrazinyl, thiophenyl, selenophenyl, benzothiophenyl, furanyl, benzofuranyl, indolyl, azaindolyl (pyrrolopyridinyl), indazolyl, benzimidazolyl, pyrazolopyridinyl, triazolopyridinyl, benzotriazolyl, benzoxazolyl, benzothiazolyl, imidazopyridinyl, isoxazolopyridinylxanthinyl, guaninyl, quinolinyl, isoquinolinyl, tetrahydroquinolinyl, quinoxalinyl, and quinazolinyl. Heteroaryl groups include fused ring systems in which all of the rings are aromatic, for example, indolyl, and fused ring systems in which only one of the rings is aromatic, for example, 2,3-dihydroindolyl. The term “halo” or “halogen” employed alone or in combination with other terms is intended to include F, Cl, Br, and I.

As used herein, the term “substituted” is intended to mean that one or more hydrogen atoms in the group indicated is replaced with one or more independently selected suitable substituents, provided that the normal valency of each atom to which the substituent/s are attached is not exceeded, and that the substitution results in a stable compound.

The term “stable” as used herein refers to compounds which possess stability sufficient to allow manufacture and which maintain their integrity for a period of time sufficient to be useful for the purposes described herein.

### Method of Making Imprinted Polymer

For the avoidance of doubt, the monomers, bead embodiments, and template embodiments discussed above may also relate to the method of making an imprinted polymer for producing an enriched cannabinoid extract from a crude Cannabis extract, method of producing an enriched cannabinoid extract from a crude non-winterized Cannabis extract, method of reducing heavy metal content in an enriched cannabinoid extract when compared to a crude Cannabis extract, method of reducing at least one pesticide residue in an enriched cannabinoid extract when compared to a crude Cannabis extract and/or method of reducing lipid extraction steps when producing an enriched cannabinoid extract from a crude Cannabis extract.

For the method of making an imprinted polymer of the invention, the polymersation is preferably carried out in the presence of an initiator, for example an oil-soluble azo initiator. The azo initiator is preferably selected from dimethyl 2,2′-azobis(2-methylpropionate), 2,2′-azobis(isobutyronitrile) (‘AIBN’), 2,2′-azobis(4-methoxy-2,4-dimethylvaleronitrile), 2,2′-azobis(2,4-dimethylvaleronitrile) or 2,2′-azobis (2-methylbutyronitrile).

Preferably the ratio of initiator to the one or more monomer(s) is about 0.001-0.2:1 by molar mass, about 0.01-0.1:1 by molar mass or about 0.01-0.05:1 by molar mass.

A suspension polymerization is one in which polymer is formed in monomer, or monomer-solvent droplets in a continuous phase that is a nonsolvent for both the monomer and the formed polymer. The method of making an imprinted polymer for producing an enriched cannabinoid extract from a crude Cannabis extract is preferably carried out as a suspension polymerization with a suspension liquid that forms the continuous phase, particularly when forming an imprinted polymer bead.

Reference to a liquid that does not dissolve the monomer(s) or polymer will also be understood to mean a liquid that poorly dissolves the monomer(s) or polymer.

Preferably the suspension liquid for the suspension polymerization is selected from one or more of water, ethyl acetate, mineral oil, toluene, xylene, cyclohexane, hexane, benzene or heptane. Preferably the solvent is selected from one or more of ethyl acetate, acetonitrile, toluene, xylene, cyclohexane, hexane, benzene, heptane. Preferably the suspension liquid is water or mineral oil. The liquid for the suspension polymerization is preferably a mixture of (1) water and (2) ethyl acetate, toluene, xylene, cyclohexane, hexane, benzene and/or heptane, (1) water and (2) ethyl acetate or (1) mineral oil and (2) acetonitrile.

Preferably a monomer pre-mix solution which is immiscible with the suspension liquid comprises the monomer, template and initiator is provided. The monomer pre-mix solution preferably comprises the monomer, template and initiator which are dissolved in at least one solvent. Preferably the monomer(s) and the template are dissolved in the solvent prior to addition of the initiator. The solvent for the monomer premix solution is preferably selected from one or more of ethyl acetate, toluene, xylene, cyclohexane, heptane, hexane, benzene, acetonitrile, preferably ethyl acetate or acetonitrile.

Preferably where the solvent is acetonitrile and the suspension liquid is mineral oil. Preferably where the solvent is ethyl acetate and the suspension liquid is water.

The monomer pre-mix solution preferably comprises:
acetonitrile, ethylene glycol dimethacrylate (EGDMA), the template and the initiator, or
acetonitrile, methacrylic acid, ethylene glycol dimethacrylate (EGDMA), the template and the initiator, or
acetonitrile, acrylic acid, ethylene glycol dimethacrylate (EGDMA), the template and the initiator, or
acetonitrile, 2-(hydroxyethyl)methacrylate, ethylene glycol dimethacrylate (EGDMA), the template and the initiator, or
acetonitrile, ethylene glycol dimethacrylate (EGDMA), the template and the initiator, or
ethyl acetate, divinylbenzene (DVB), styrene, the template and the initiator, or
ethyl acetate, tert-butylacryamide (TBA), ethylene glycol dimethacrylate (EGDMA), the template and the initiator, or
ethyl acetate, styrene, ethylene glycol dimethacrylate (EGDMA), the template and the initiator.

It believed the relatively non-polar cannabinoids have a greater affinity for relatively non-polar MIPs and that the non-polar MIPs are preferably prepared in a polar suspension liquid (for example water) rather than a non-polar suspension liquid (such as mineral oil).

There is preferably a greater volume of suspension liquid than monomer pre-mix solution. The ratio of suspension liquid and monomer pre-mix solution is preferably about 0.5-50:1 by volume.

Preferably the ratio of water as the suspension liquid and monomer pre-mix solution comprising ethyl acetate is about 100-10:1 by volume, or about 20-60:1 by volume, or about 30-50:1 by volume.

Preferably the ratio of mineral oil as the suspension liquid and monomer pre-mix solution comprising acetonitrile is about 0.5-20:1 by volume, or about 0.5-10:1 by volume, or about 0.6-8:1 by volume.

In the method of making the imprinted polymer of the invention, the liquid is preferably agitated, such that the polymer forms in beads. Preferably the liquid is agitated at about 100-1500 RPM (revolutions per minute) or about 200-1000 RPM. Preferably the liquid is agitated for at least about 8 hours, at least about 10 hours, at least about 12 hours or for about 10-24 hours. Agitation is preferably carried out for a sufficient period to substantially complete the polymerization reaction. Once the polymerization reaction is completed, agitating for longer periods of time may lower the production rate.

Preferably the monomer(s) and the template are dissolved in the solvent and added to the suspension liquid. In some embodiments the monomer(s), the template and the initiator are dissolved in the solvent and added to the suspension liquid, preferably the monomer(s) and the template are dissolved in the solvent prior to addition of the initiator.

The polymerization is preferably maintained at a temperature of between about 40-80° C. or preferably about 50-70° C.

Method of making an imprinted polymer preferably comprises at least partially removing the template molecule from the imprinted polymer. Preferably a solvent is used to at least partially remove the template molecule from the imprinted polymer. The template will be removed prior to contacting the imprinted polymer with the crude Cannabis extract.

### Method of Producing an Enriched Cannabinoid Extract

As discussed above, the methods described herein comprise the steps of: a) contacting an imprinted polymer with the crude Cannabis extract wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers b) eluting at least the cannabinoids from the imprinted polymer with an elution solvent to produce a cannabinoid extract comprising one or more cannabinoid(s).

For the avoidance of doubt the embodiments discussed above relating to the monomers, beads, templates and methods to make the imprinted polymer may also apply the a method of producing an enriched cannabinoid extract from a crude Cannabis extract, method of producing an enriched cannabinoid extract from a crude non-winterized Cannabis extract, method of reducing heavy metal content in an enriched cannabinoid extract when compared to a crude Cannabis extract, method of reducing at least one pesticide residue in an enriched cannabinoid extract when compared to a crude Cannabis extract and/or method of reducing lipid extraction steps when producing an enriched cannabinoid extract from a crude Cannabis extract.

Reference to “eluting”, “elution” should be taken refer to extracting one material from another by washing with a solvent. It should not be taken to be limited to chromatography but can include chromatography.

In the method of producing an enriched cannabinoid extract from a crude Cannabis extract/method of producing an enriched cannabinoid extract from a crude non-winterized Cannabis extract/method of reducing heavy metal content in an enriched cannabinoid extract when compared to a crude Cannabis extract/method of reducing at least one pesticide residue in an enriched cannabinoid extract when compared to a crude Cannabis extract and/or method of reducing lipid extraction steps when producing an enriched cannabinoid extract from a crude Cannabis extract, step (a) may be repeated before moving to step (b) and (b) may be repeated. Where they are repeated, they are repeated between at least 1-2 times, for example 1 and 10 times, however, this will be dependent on the desired outcome and amount of crude Cannabis extract. Repeating steps (a) and (b) may allow the total cannabinoid concentration and/or recovery to be increased. These options are discussed further below.

Step (a) is optionally followed by collection of the crude Cannabis extract that has been contacted with the imprinted polymer to give a Cannabis extract. The Cannabis extract is optionally used in step (a) in place of the crude Cannabis extract. For example the crude Cannabis extract is contacted with the imprinted polymer more than once, by collecting and re-contacting one or more times. In this way the crude Cannabis extract can be cycled over the imprinted polymer one or more further times. It is believed this allows greater uptake/binding/capture or association of the cannabinoid(s) and/or other desirable components such as terpenoids or terpenes, with the imprinted polymer and may improve recovery following the elution step. In this option the elution step follows the contacting of the crude Cannabis extract/Cannabis extract with the imprinted polymer.

The method preferably produces an enriched cannabinoid extract that is not reconstituted. The method is suitable for use without having to add any additional cannabinoids, so in some embodiments the method comprises not adding cannabinoids, cannabinoids or terpenes.

Following the method of producing an enriched cannabinoid extract from a crude Cannabis extract the enriched cannabinoid extract has a greater proportion of at least one cannabinoid than the crude Cannabis extract.

Where the crude Cannabis extract comprises cannabinoids in acid form (i.e. carboxylic acids, for example THCA and/or CBDA), the enriched cannabinoid extract preferably has the same or increased proportion of cannabinoid in acid form over the crude Cannabis extract. This is particularly advantageous as prior art methods such as distillation may convert the acids to non-acid forms.

In some embodiments the enriched cannabinoid extract has an increased proportion of CBD and/or CBG over the crude Cannabis extract.

In some cases the crude Cannabis extract can comprise heavy metals and/or pesticides, in some cases at levels that are not safe for consumption or are not saleable under the regulatory regimes in some jurisdictions. Advantageously, in some embodiments, the enriched cannabinoid extract obtained following the method of the present invention preferably has a reduced proportion of heavy metals and/or pesticides over the crude Cannabis extract. Examples of heavy metals in the crude Cannabis extract can include one or more of arsenic, cadmium, chromium, copper, lead, nickel and/or zinc. The enriched Cannabis extract may have about 50%-100% by mass reduction in one or more heavy metals when compared to the crude Cannabis extract, or about 60%-100% by mass reduction, or about 70%-100% by mass reduction, or about 80%-100% by mass, or about 90%-100% by mass reduction, or about 95%-100% by mass reduction, or about 98%-100% by mass reduction in one or more heavy metals.

In particular there may be a reduction in arsenic in the enriched cannabinoid extract when compared to the crude Cannabis extract, for example about 50%-100% by mass reduction, or about 60%-100% by mass reduction, or about 70%-100% by mass reduction, or about 80%-100% by mass reduction, or about 90%-100% by mass reduction.

There may also or alternatively be a reduction in lead in the enriched cannabinoid extract when compared to the crude Cannabis extract, for example about 50%-100% by mass reduction, or about 60%-100% by mass reduction, or about 70%-100% by mass reduction, or about 80%-100% by mass reduction, or about 90%-100% by mass reduction.

The method of producing an enriched cannabinoid extract from a crude Cannabis extract described herein may therefore also or alternatively be a method of reducing heavy metal content in an enriched cannabinoid extract when compared to a crude Cannabis extract. An further aspect of the invention is a method of reducing heavy metal content in an enriched cannabinoid extract when compared to a crude Cannabis extract, the method comprising the steps of: a) contacting an imprinted polymer with the crude Cannabis extract, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers; b) eluting the cannabinoids from the imprinted polymer with an elution solvent to produce the enriched cannabinoid extract comprising one or more cannabinoid(s).

Examples of pesticides include myclobutanil, pyrimentanil, carbaryl, permethrin, diazinon and/or ethoprophos. For example, the reduction in one or more pesticides in the enriched Cannabis extract compared to the crude Cannabis extract may be about 30%-100% by mass, or about 40%-100% by mass, or about 50%-100% by mass, or about 60%-100% by mass. The method of producing an enriched cannabinoid extract from a crude Cannabis extract described herein may therefore also or alternatively be a method of reducing at least one pesticide residue in an enriched cannabinoid extract when compared to a crude Cannabis extract. An further aspect of the invention is a method of reducing at least one pesticide residue in an enriched cannabinoid extract when compared to a crude Cannabis extract, the method comprising the steps of: a) contacting an imprinted polymer with the crude Cannabis extract, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers; b) eluting the cannabinoids from the imprinted polymer with an elution solvent to produce the enriched cannabinoid extract comprising one or more cannabinoid(s).

The method of the invention preferably allows for varying the proportion of one or more terpene or the total proportion content of terpene in the enriched cannabinoid extract over the crude Cannabis extract. The method of the invention preferably allows for varying the proportion of one or more terpenoid(s) or the total proportion content of terpenoid in the enriched cannabinoid extract over the crude Cannabis extract. The method of the invention preferably allows for varying the proportion of one or more terpene(s) or the total proportion of terpenes in the enriched cannabinoid extract over the crude Cannabis extract. In some cases, the proportion of one or more terpenes or the total proportion of terpenes in the enriched cannabinoid extract is increased over the crude Cannabis extract. Alternatively, the proportion of one or more terpenes or the total proportion of terpenes in the enriched cannabinoid extract is decreased over the crude Cannabis extract. The proportion of the one or more terpenes or total proportion of terpenes in the enriched cannabinoid extract may be varied or controlled by selection of the conditions for the method, for example the monomer the imprinted polymer is formed from. For example, preferably where EGDMA/TBA monomers are used, the terpene proportion is decreased, or preferably where DVB/styrene monomers are used the terpene proportion is increased. Preferably the terpene is selected from one or more of linalool, caryophyllene oxide, guaiol, alpha-bisabolol, beta-caryophyllene, beta-myrcene, D-limonene, alpha-humulene, trans-nerolidol, geraniol, valencene, terpineol, borneol, camphene, delta-3-carene, eucalyptol, alpha-pinene, beta-pinene. Preferably the terpene that increases is selected from one or more of linalool, caryophyllene oxide, guaiol, alpha-bisabolol, beta-caryophyllene, alpha-humulene, trans-nerolidol. Preferably the terpene that decreases is selected from one or more of beta-myrcene, linalool, guaiol, beta-caryophyllene, D-limonene, alpha-humulene. Alternatively, the proportion of one or more terpenes or the total proportion of terpenes remains the same in the enriched Cannabis extract compared to the crude Cannabis extract. Some prior art methods undesirably remove or alter the profile of the terpene(s) present in the crude Cannabis extract. Advantageously, in some embodiments the method of the present invention produces an enriched cannabinoid extract with substantially the same terpene proportion or proportion of one or more specific terpenes as the crude Cannabis extract. Preferably the terpene that remains approximately the same is selected from one or more of caryophyllene oxide, alpha-bisabolol, trans-nerolidol.

### Reduction in Lipid and Dispersal Step in Methods

The methods of the invention is flexible enough to take a range of starting material (crude Cannabis extract), for example that has been winterized or not (i.e. with higher lipid content), carboxylated or decarboxylated.

The method of producing an enriched cannabinoid extract from a crude Cannabis extract described herein may therefore also or alternatively be a method of reducing lipid extraction steps when producing an enriched cannabinoid extract from a crude Cannabis extract and/or a method of producing an enriched cannabinoid extract from a crude non-winterized Cannabis extract. An further aspect of the invention is a method of reducing lipid extraction steps when producing an enriched cannabinoid extract from a crude Cannabis extract, the method comprising the steps of: a) contacting an imprinted polymer with the crude Cannabis extract containing lipid, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers; b) eluting the cannabinoids from the imprinted polymer with an elution solvent to produce the enriched cannabinoid extract comprising one or more cannabinoid(s). A further alternative aspect of the invention is a method of producing an enriched cannabinoid extract from a crude non-winterized Cannabis extract, the method comprising the steps of: a) contacting an imprinted polymer with the crude non-winterized Cannabis extract, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers; b) eluting the cannabinoids from the imprinted polymer with an elution solvent to produce the enriched cannabinoid extract comprising one or more cannabinoid(s). Preferably in these aspects the imprinted polymer is contacted with the crude non-winterized Cannabis extract or the crude Cannabis extract containing lipid, in the form of an emulsion and/or solution.

The crude Cannabis extract is preferably dispersed in a suitable liquid prior to contacting the imprinted polymer with the crude Cannabis extract, for example the crude Cannabis extract is in the form of a solution and/or emulsion when it is introduced to the imprinted polymer. Examples of suitable liquids are ethanol, water, methanol, ethyl acetate, isopropyl alcohol, acetonitrile, acetone, THF or mixtures thereof, preferably a mixture of water and ethanol.

It is particularly preferred that the crude extract is substantially uniformly dispersed in solution/emulsion prior to contacting the imprinted polymer, for example, particles are broken up and evenly dispersed, in particular the fats and/waxes (lipids). This can be achieved for example by one or more of sonication or ultrasonication, and/or high shear mixing. The dispersal step is preferably carried out for a sufficient time so as to produce a substantially uniformly dispersed emulsion and/or a solution. It is considered the sonication/ultrasonication is likely to be preferred over high shear mixing or alternatively both sonication/ultrasonication and high shear mixing could be used where the crude Cannabis extract is of low quality, for example crude Cannabis extract with high levels of lipids (for example fats and/or waxes), and/or where large quantities of crude Cannabis extract are being processed.

It is preferred that about 1 gram of the crude extract is dissolved/emulsified in about 5 to 100 ml of the one or more liquids, about 5 to 80 ml of the one or more liquids, about 5 to 60 ml of the one or more liquids, or about 5 to 50 ml of the one or more liquids.

The dispersal step is particularly preferred when the crude Cannabis extract has not previously undergone winterization. The dispersal step allows for the method to be carried out on a crude Cannabis extract that has not undergone winterisation or for example contains at least 1% lipid, at least 2% lipid, at least 3% lipid, at least 4% lipid or at least 5% lipid or about 1% to 60% lipid, about 2% to 60% lipid, about 3% to 60% lipid, about 4% to 60% lipid, or about 5% to 60% lipid, or 1% fat or wax, at least 2% fat or wax, at least 3% fat or wax, at least 4% fat or wax or at least 5% fat or wax or about 1% to 60% fat or wax, about 2% to 60% fat or wax, about 3% to 60% fat or wax, about 4% to 60% fat or wax, or about 5% to 60% fat or wax.

The present invention offers the ability to avoid or significantly reduce a winterization step, for example winterization can be done under milder conditions, for a shorter time or at higher temperatures. Avoidance of a winterization step is a significant improvement, as winterization has previously made purification methods more time consuming, energy-intensive and logistically difficult. Winterization involves dissolving the extract in solvent, then chilling for 24-48 hours to solidify the fats/waxes/other lipids, which are then filtered off. This may be repeated if necessary for complete reduction in fat/wax/other lipid content.

As noted above, step (a) is optionally followed by collection of the crude Cannabis extract that has been contacted with the imprinted polymer to give a Cannabis extract, preferably the Cannabis extract is used in step (a) in place of the crude Cannabis extract. Where the emulsion and/or solution of the crude Cannabis extract is formed, the Cannabis extract that is collected following contact with the imprinted polymer is also in the form of a emulsion and/or solution which is optimally used in step (a) in place of the crude Cannabis extract, for example the emulsion and/solution of the crude Cannabis extract is cycled over the imprinted polymer more than once prior to the eluting step (b). The imprinted polymer is preferably contacted with the emulsion and/or solution more than once, for example between 2 and 20 times prior to step (b). The emulsion and/or solution is preferably run through or over the imprinted polymer collected and then run through or over the imprinted polymer one or more times, for example the emulsion and/or solution is cycled over or through the imprinted polymer more than once. It is believed this allows greater uptake/binding/capture or association of the cannabinoid(s) and/or other desirable components such as terpenoids or terpenes, with the imprinted polymer and may improve recovery following the elution step.

Rinse Step in Method of Producing an Enriched Cannabinoid Extract from a Crude Cannabis Extract

In the methods described herein there is optionally a rinse step following step (a) and prior to step (b). The rinse step preferably comprises washing the imprinted polymer with a rinse liquid to remove at least a portion of undesired components from the crude extract. For example, the desired cannabinoids and in some cases terpenes are held by, bound to or otherwise associated with the imprinted polymer and the rinse step removes undesired components from the imprinted polymer. A rinse liquid is a liquid that is used in the rinse step which is capable of remove at least a portion of undesired components from the crude extract from the imprinted polymer.

Preferably the rinse liquid comprises one or more of water, ethanol, methanol, ethyl acetate, isopropyl alcohol, acetonitrile, acetone, tetrahydrofuran (THF).

Preferably the rinse solution is up to 50% ethanol in water, preferably about 1-50% ethanol in water, preferably about 20% ethanol in water (by volume).

Preferably the rinse solution is more polar than the elution solvent used in step (b).

In some embodiments the rinse liquid after it has been used (comprising some cannabinoid and some non-cannabinoids plus the rinse liquid) is retained for further processing (for example to make the emulsion and/or solution of the crude Cannabis extract, or for separate purification).

Elution Step in Method of Producing an Enriched Cannabinoid Extract from a Crude Cannabis Extract

Step (b) of the methods described here comprises eluting at least the cannabinoids from the imprinted polymer with an elution solvent to produce an enriched cannabinoid extract comprising one or more cannabinoid(s). In some embodiments terpenes are also eluted.

An elution solvent is a solvent used in the elution step that is capable of eluting at least a portion of the cannabinoids from the imprinted polymer.

Once it has been eluted through and/or over the imprinted polymer the eluent comprises the cannabinoids and in some cases other components, such as terpenes. Solvent can optionally be evaporated from the eluent to give the enriched cannabinoid extract. The solvent that is evaporated can optionally be at least partially recovered and may optionally be reused in the method.

Alternatively the enriched cannabinoid extract from step (b) is collected and used in place of the crude Cannabis extract in step (a) and step (a) and step (b) are repeated using the enriched cannabinoid extract, for example using new eluent. Between cycles the eluent can be evaporated (reduced) or diluted (increased) to a desired concentration or solvent mixture before being recontacted with the imprinted polymer. This is believed to increase the potency of the resulting enriched cannabinoid extract. For example, the method is alternatively as follows the steps of: a) contacting an imprinted polymer with the crude Cannabis extract wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers b) eluting at least the cannabinoids from the imprinted polymer with an elution solvent to produce an enriched cannabinoid extract comprising one or more cannabinoid(s), c) contacting the imprinted polymer with the enriched cannabinoid extract and d) eluting at least the cannabinoids from the imprinted polymer with an elution solvent to produce a further enriched cannabinoid extract, wherein steps c) and d) can be repeated one or more times as desired.

Where the Cannabis extract from step (b) is contacted with the imprinted polymer again, the contacting step can be repeated one or more times before eluting again, for example the Cannabis extract from step (b) is collected and re-contacted with the imprinted polymer one or more times before eluting.

The elution solvent preferably comprises one or more of ethanol, methanol, ethyl acetate, isopropyl alcohol, acetonitrile, acetone or THF solvents. Solvent(s) that are considered food safe are preferred. Preferably the elution solvent comprises or consists of ethanol.

The elution solvent may be a mixture of more than one solvent, optionally the proportions or mixtures of the more than one solvent may change over the course of step (b) and/or can be introduced in portions or continuously. The elution solvent may be passed over the imprinted polymer at varying speeds over the course of the elution step.

The eluent can be collected in one portion or more than one portion. For example, the eluent can be collected in different fractions over the course of the method. The different fractions may have differing proportions of one or more cannabinoid(s) or one or more terpenes. Optionally desired fractions can be combined to obtain an extract comprising desired cannabinoids/profile.

The imprinted polymer may optionally be conditioned by performing steps a) and b) one or more times prior to collecting the elution solvent to produce the enriched cannabinoid extract comprising one or more cannabinoid(s). Preferably the imprinted polymer is conditioned by performing steps a) and b) between one and five times, preferably one and three times, prior to collecting the elution solvent to produce the enriched cannabinoid extract comprising one or more cannabinoid(s). The inventors have observed the initial elution of a new previously unused imprinted polymer in some cases gives inconsistent or lower cannabinoid potency. While not wishing to be bound by theory, it is believed on the initial binding the cannabinoid(s) and or other components (for example terpenes) fill the internal binding sites which are more difficult to elute. However, after the conditioning phase the levels of bound and eluted cannabinoid(s) and or other components become more consistent.

### Regenerating Step

The method of producing an enriched cannabinoid extract from a crude Cannabis extract/method of producing an enriched cannabinoid extract from a crude non-winterized Cannabis extract/method of reducing heavy metal content in an enriched cannabinoid extract when compared to a crude Cannabis extract/method of reducing at least one pesticide residue in an enriched cannabinoid extract when compared to a crude Cannabis extract and/or method of reducing lipid extraction steps when producing an enriched cannabinoid extract from a crude Cannabis extract can optionally further comprise a regeneration step. The regeneration step is preferably done after step (b), but may not be done after every step (b) where step (a) and (b) are repeated. The regeneration step comprises cleaning the imprinted polymer with a regeneration solvent to remove unwanted components of the crude extract that may foul the imprinted polymer. For example, fats and waxes or other lipids may build up over time which can be removed using the regeneration step. A regeneration solvent is a solvent that is used in the regeneration step which is capable of cleaning the imprinted polymer. The regeneration solvents used for the regeneration step are selected to dissolve the unwanted components, for example, where the unwanted components are lipids (for example fats and/or waxes) the regeneration solvent is relatively non-polar solvent (compared to the elution solvent), for example isopropyl alcohol, acetone, an alkane (for example hexane). The regeneration step can optionally further comprise washing with a further solvent, for example the solvent elution solvent or rinse liquid to remove the regeneration solvent.

### Crude Cannabis Extract

The crude Cannabis extract is preferably produced by extraction of plant matter with a solvent. The crude Cannabis extract is preferably produced by extraction with supercritical carbon dioxide, subcritical carbon dioxide, ethanol, one or more hydrocarbons (for example propane, butane, hexane). The quality and composition of the plant matter (for example leaves with low cannabinoid content or buds with high cannabinoid content) will affect the quality and composition of the crude extract. The crude Cannabis extract comprises a substantial proportion of at least one non-cannabinoid material selected from one or more of waxes, fats, lipids, wax esters, plant pigments, glycerides, unsaturated fatty acids, one or more pesticide contaminants, one or more heavy metal contaminants, terpenes, carotenes, flavonoids. Examples of heavy metals include arsenic, cadmium, chromium, copper, lead, nickel and zinc. Examples of pesticides include myclobutanil, pyrimethanil, carbaryl, permethrin, diazinon and/or ethoprophos.

Where there is reference to cannabis, this can include either marijuana and/or hemp. Hemp contains much the same makeup as marijuana, but usually with far less THC, and often lower concentrations of the other cannabinoids. The concentrations of cannabinoids (and in some instances terpenes or terpenoids) are generally far less in hemp, hemp based Cannabis extracts are more difficult to process and sometimes this additional processing can becomes uneconomical. There is surging interest and demand in cannabis/hemp products as regulations around these products are relaxed, which has led to a proliferation of new growers, resulting in large fluctuations in quality of starting plant material. This can result in lower quality crude extracts when the material is processed, which in turn requires more purification, again which may not be economical with prior art technologies.

The methods of the invention are however flexible enough to take a range of starting material (crude Cannabis extract), for example that has been winterized or not (i.e. with higher lipid content), carboxylated or decarboxylated and for example may have had previous purification steps.

Preferably the crude Cannabis extract used in the method of the invention comprises at least 1% lipid, at least 2% lipid, at least 3% lipid, at least 4% lipid, at least 5% lipid. Preferably the crude Cannabis extract used in the method of the invention comprises about 1% to 60% lipid, about 2% to 60% lipid, about 3% to 60% lipid, about 4% to 60% lipid, about 5% to 60% lipid. Preferably the crude Cannabis extract used in the method of the invention comprises about 1% to 50% lipids, about 2% to 50% lipid, about 3% to 50% lipid, about 4% to 50% lipid, about 5% to 50% lipid.

The crude Cannabis extract used in the method of the invention preferably is not winterized, or otherwise removed of lipids (e.g. fats and waxes and other lipids including wax esters, glycerides, unsaturated fatty acids). Advantageously, the method of the invention, substantially removes the fats and waxes in the crude Cannabis extract without the need for a separate step (such as winterization).

Non-winterized crude Cannabis extract is typically solid/waxy in consistency. Winterized crude extract is usually a thick viscous liquid, for example a viscosity similar to honey. A comparison of the properties of the crude Cannabis extract (input) and the enriched cannabinoid extract produced by embodiments of the method of the invention is shown in Table 1.

TABLE 1

| | | Output after processing with |
|---|---|---|
| | Crude Input | MIPs |
| Cannabinoid | Typically 50-60%, when | At least 65% cannabinoids |
| content | extracted from good quality | |
| | plant material | |
| Fat and/or | Can be up to about 50% if | Substantially free from fats |
| wax content | from poor quality starting | and waxes |
| | plant material, but | |
| | typically in the range of | |
| | about 5-15% depending on | |
| | plant material input and | |
| | extraction method | |
| Heavy metal | Present in the crude extract | Substantially free from |
| content | if they were bio- | heavy metals |
| | accumulated in the plant | |
| Pesticide | Present in the crude extract | Substantially free from |
| content | if they were present in the | pesticides |
| | plant | |
| Colour | Black/brown | Amber/orange |
| Viscosity | Highly viscous or solid | Reduced viscosity |
| Taste and | Not palatable, bitter | Positive flavour and aroma |
| aroma | | profile, less bitter |

### Enriched Cannabinoid Extract Product and Terpenes

The enriched cannabinoid extract preferably has a greater proportion of at least one cannabinoid than the crude Cannabis extract. In one aspect the invention provides an enriched cannabinoid extract comprising greater than 65% combined mass of one or more cannabinoid(s), at least one cannabinoid selected from the group CBD, THC, CBN, CBND, CBC, THCV, CBL, CBE and CBDV; greater than about 0.03% by weight CBG; and at least one terpene, selected from linalool, caryophyllene oxide, guaiol, alpha-bisabolol, beta-caryophyllene, alpha-humulene, trans-nerolidol.

Preferably the enriched cannabinoid extract comprises greater than 1.15% by weight CBG, greater than 2% by weight CBG, or between about 2-10% by weight CBG, or about 2-5% by weight CBG.

Preferably the enriched cannabinoid extract comprises greater than about 70% combined mass of one or more cannabinoid(s), or greater than about 75% combined mass of one or more cannabinoid(s) or greater than about 80% combined mass of one or more cannabinoid(s).

Preferably the enriched cannabinoid extract comprises at least one cannabinoid acid selected from the group CBDA, THCA, CBGA, CBCA, CBLA, CBEA-A and CBEA-B, preferably at least two cannabinoid acids selected from the group CBDA, THCA, CBGA, CBCA, CBLA, CBEA-A and CBEA-B. Preferably the enriched cannabinoid extract comprises CBDA.

Preferably the enriched cannabinoid extract comprises less than 30%, preferably less than 25%, and more preferably less than 15% content by weight of molecules found within Cannabis plant material that fall outside the size range of about 100 to 450 grams per mol.

Preferably the enriched cannabinoid extract comprises less than 0.1% by mass heavy metals, less than 0.05% by mass heavy metals, less than 0.01% by mass heavy metals, less than 0.001% by mass heavy metals Preferably the enriched cannabinoid extract is substantially free from heavy metals. Examples of heavy metals include one or more of arsenic, cadmium, chromium, copper, lead, nickel and/or zinc.

Preferably the enriched cannabinoid extract comprises less than 0.1% by mass pesticide residue, less than 0.05% by mass pesticide residue, less than 0.01% by mass pesticide residue. Preferably the enriched cannabinoid extract is substantially free from pesticide residue. For example, the enriched cannabinoid extract may comprise less than 0.1% by mass of a pesticide residue, or less than 0.05% by mass total pesticide residue, less than 0.01% by mass a pesticide residue or less than 0.01 by mass total pesticide residue. For example, the enriched cannabinoid extract is substantially free from a pesticide residue or is substantially free from total pesticide residue. Examples of pesticides include one or more myclobutanil, pyrimethanil, carbaryl, permethrin, diazinon and/or ethoprophos.

Preferably the enriched cannabinoid extract comprises more than one terpene.

Preferably the enriched cannabinoid extract comprises linalool, caryophyllene oxide, guaiol, alpha-bisabolol, beta-caryophyllene, alpha-humulene and/or trans-nerolidol.

Preferably the enriched cannabinoid extract comprises less than about 2% by mass non-cannabinoid molecules that cause bitter taste, less than about 1% by mass non-cannabinoid molecules that cause bitter taste, less than about 0.5% by mass non-cannabinoid molecules that cause bitter taste, less than about 0.1% by mass non-cannabinoid molecules that cause bitter taste, less than about 0.1% by mass non-cannabinoid molecules that cause bitter taste. Preferably the enriched cannabinoid extract is substantially free from non-cannabinoid molecules that cause responsible for bitter taste.

Preferably enriched cannabinoid extract comprises less than 10% lipid, less than 5% lipid, less than 1% lipid, less than 0.05% lipid, less than 0.05% lipid, less than 0.01% lipid. Preferably the enriched cannabinoid extract comprises substantially no lipid. Preferably enriched cannabinoid extract comprises about 0-10% lipid, about 0-5% lipid, about 0-1% lipid, about 0-0.05% lipid, about 0-0.05% lipid, about 0-0.01% lipid. The lipid is preferably fat and/or wax.

Where, in the foregoing description reference has been made to integers or components having known equivalents thereof, those integers are herein incorporated as if individually set forth.

Although the present disclosure has been described in terms of certain embodiments, other embodiments apparent to those of ordinary skill in the art also are within the scope of this disclosure. Thus, various changes and modifications may be made without departing from the spirit and scope of the disclosure. For instance, various components may be repositioned as desired. Moreover, not all of the features, aspects and advantages are necessarily required to practice the present disclosure. Accordingly, the scope of the present disclosure is intended to be defined only by the claims that follow.

### EXAMPLES

### 1. Synthesis of Imprinted Beads

The synthesis of molecularly imprinted polymer (MIP) beads is carried out via a suspension polymerization mechanism in a suspension liquid. The suspension liquid is charged into the reaction vessel and heated to between 50-70° C. A monomer pre-mix solution containing the non-continuous solvent, the monomer, the cross-linker and the template molecule is prepared and agitated. This solution was left to mix until complete dissolution of all reagents. Next, the initiator is added to the pre-mix and the premix solution is dosed into the reactor.

The premix solution is suspended in the suspension fluid (continuous solvent or suspension liquid) and agitated inside the reactor to form droplets. An agitation rate of between 200-1000 RPM is used. The reaction mixture is held until the reaction is substantially complete before cooling down to room temperature and discharging. The MIP beads are discharged from the reactor and cleaned using a solvent to remove the template molecule and any unreacted components. The cleaning step may be repeated until the MIP beads are sufficiently free from residual template molecule and unreacted components. After the final cleaning step, the MIP beads are collected and dried. The resulting MIP beads may be between 0.1 to 10 mm, but usually between 0.5 mm-3 mm in diameter. The parameters for synthesis of a range of imprinted polymer beads is shown in Table 2. The beads have been produced at scales from <10 g to 100 kg (output).

TABLE 2

| | | | | | | | |
|---|---|---|---|---|---|---|---|
| Example 1 | Example 2 | Example 3 | Example 4 | Example 5 | Example 6 | Example 7 | Example 8 |

INPUT

| | | | | | | | | |
|---|---|---|---|---|---|---|---|---|
| Solvent* | Acetonitrile: | Acetonitrile: | Acetonitrile: | Ethyl | Acetonitrile: | Acetonitrile: | Ethyl | Ethyl |
| | 5.02 | 7.67 | 4.20; | acetate: | 5.07 | 5.13 | acetate: | acetate: |
| | | | Ethanol: | 2.03 | | | 2.69 | 2.70 |
| | | | 0.75 | | | | | |
| Monomers* | Methacrylic | Acrylic | (Hydroxyethyl) | Styrene: | (Hydroxyethyl) | EGDMA: | TBA: | Styrene: |
| | acid: | acid: | methacrylate: | 0.17 | methacrylate: | 1.00 | 0.15 | 0.69 |
| | (0.44) | 1.00 | 0.22 | Divinyl | 1.00 | | EGDMA: | EGDMA: |
| | EGDMA: | EGDMA: | EGDMA: | benzene: | EGDMA: | | 1.00 | 1.00 |
| | (1.00) | 0.64 | 1.00 | 1.00 | 0.98 | | | |
| Template* | CBD: | Catechin | Catechin | Catechin | Olivetol: | CBD: | Catechin | Catechin |
| | 0.03 | hydrate: | hydrate: | hydrate: | 0.08 | 0.01 | hydrate: | hydrate: |
| | | 0.03 | 0.01 | 0.02 | | | 0.01 | 0.01 |
| Ratio template | 1:27 | 1:53 | 1:108 | 1:75 | 1:25 | 1:100 | 1:115 | 1:160 |
| to total | | | | | | | | |
| monomers | | | | | | | | |
| (mols) | | | | | | | | |
| Initiator* | 0.13 | 0.04 | 0.01 | 0.01 | 0.003 | 0.13 | 0.03 | 0.06 |
| Ratio initiator | 0.074:1 | 0.023:1 | 0.077:1 | 0.013:1 | 0.080:1 | 0.131:1 | 0.028:1 | 0.036:1 |
| to total | | | | | | | | |
| monomers | | | | | | | | |
| (mol) | | | | | | | | |
| Suspension | Mineral oil: | Mineral oil: | Mineral oil: | Deionized | Mineral oil: | Mineral oil: | Deionized | Water: 35:1 |
| fluid** | 0.76:1 | 1.49:1 | 0.80:1 | water: 36:1 | 0.88:1 | 5.84:1 | water: 41:1 | |

PROCESSING PARAMETERS

| | | | | | | | | |
|---|---|---|---|---|---|---|---|---|
| Temperature (° C.) | 70-80 | 70-80 | 60-70 | 50-70 | 50-60 | 70-80 | 60-70 | 60-70 |
| Agitation rate | 300 | 400 | 290 | 900 | 400 | 300 | 300 | 250 |
| (RPM) | | | | | | | | |

OUTPUT

| | | | | | | | | |
|---|---|---|---|---|---|---|---|---|
| Yield | 70-80% | 70-80% | 70-80% | 70-80% | 70-80% | 70-80% | 80-90% | 70-80% |
| Average particle | 0.5-2 mm | 0.5-1 mm | 1-2 mm | 0.2-1 mm | 0.5-1 mm | 0.5-2 mm | 1-2 mm | 2-3 mm |
| size | | | | | | | | |

*Ratios calculated based on primary monomer.
Reactant phase is made up of the solvent, monomers, template and initiator.
Continuous phase/suspension fluid suspends the reactant phase during polymerization.
**Suspension fluid volume given as a ratio of suspension fluid to reactant phase by moles.

### 2. Cannabinoid Extraction Using MIPS

Crude Cannabis extracts were sonicated in ethanol/water solution then were passed through columns of MIP beads to test the ability of the beads to enrich/purify/concentrate cannabinoids in the extract.

The general parameters of the experiment are shown in Table 3.

TABLE 3

| | |
|---|---|
| Column | EGDMA/TBA (catechin template) Beads (0.5-1.5 mm |
| | diameter), 233.45 g |
| Flow rate | 100 mL/min |
| (both columns) | |
| Extract | 50 g of decarboxylated non-winterized extract, 1 L of |
| Solution | Ethanol, and 1 L of H₂O Sonicated to form a solution. |
| | (~2.5 g of extract per 100 mL) |
| Rinse | 1.6 L of H₂O and 0.4 L of Ethanol (20% ethanol |
| Solution | Solution) |
| Elution | 2 L of Ethanol |

2 L of the extract solution was pumped through the column to introduce the crude Cannabis extract into the column. 2 L of the 20% ethanol rinse was then pumped through the column at 100 mL/min to rinse away undesired components from the extract. 2 L of ethanol eluent was pumped through the column to obtain the cannabinoids. This ethanol eluent solution was collected for reduction. The eluent was reduced using a rotary evaporator (rotovap) then vacuum and heat applied to remove any remaining ethanol. A sample of the eluent concentrate was taken and sent for analysis.

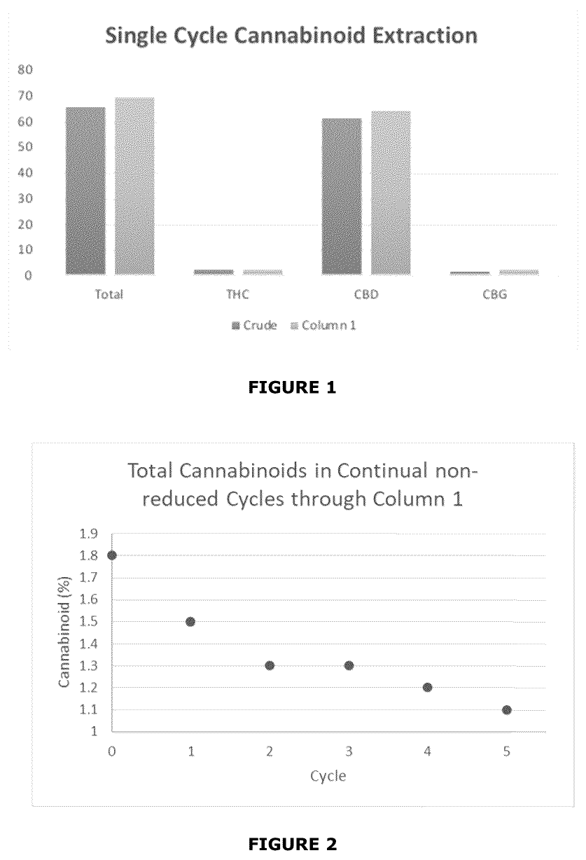

The results are shown in Table 4 and FIG. 1. The results show that the MIP beads bind a significant proportion of the cannabinoids present in the starting sample (reference). The MIP also concentrates CBG and CBD to levels beyond those detected in the starting sample.

The crude extract used in this experiment was decarboxylated extract, i.e. it was heated to convert cannabinoid acids into cannabinoids. The crude material therefore did not contain cannabinoid acid(s), such as CBDA or THCA, and as expected the products (enriched cannabinoid extract) did not contain cannabinoid acid(s) either.

TABLE 4

Cannabinoid %

| | | Column |
|---|---|---|
| | Crude extract | (EGDMA/TBA) |
| Total | 65.3 | 69.1 |
| THC | 2.58 | 2.27 |
| CBD | 61.09 | 64.53 |
| CBG | 1.66 | 2.32 |

### 3. Cycling Matrix to Maximize Cannabinoid Capture

### 3.1 Cycling of Decarboxylated Extract

The general parameters of the experiment are shown in Table 5.

TABLE 5

| | |
|---|---|
| Column | EGDMA/TBA (catechin template) Beads (0.5-1.5 mm |
| | diameter), 233.45 g |
| Flow rate | 100 mL/min |
| Extract | 50 g of decarboxylated, non-winterized extract, 1 L of |
| Solution | Ethanol, and 1 L of H₂O Sonicated to form a solution. |
| | (~2.5 g of extract per 100 mL) |
| Rinse | 1.6 L of H₂O and 0.4 L of Ethanol (20% ethanol |
| Solution | Solution) |
| Elution | 2 L of Ethanol |

Step 1: 2 L of the Extract solution was pumped through the column and collected. Step 2: a sample of the extract solution was taken for analysis. Steps 1-2 were repeated 4 more times, i.e. the same extract solution was repeatedly put through the column to increase cannabinoid yield.

2 L of the 20% ethanol rinse was then pumped through the Column.

2 L of ethanol eluent was pumped through the column. The eluent was reduced using a rotary evaporator, then vacuum and heat applied to remove any remaining ethanol. The samples of extract and the eluent concentrate were taken and sent for analysis.

The results for the decarboxylated extract are shown in Table 6 and FIG. 2. The “matrix” in Table 6 is the emulsion/solution of crude Cannabis extract.

The results demonstrate that cycling the emulsion/solution of the crude Cannabis extract maximizes cannabinoid capture, enabling close to 9900 recovery.

The extract used in this experiment was decarboxylated extract, i.e. it was heated to convert cannabinoid acids into cannabinoids. The reference material and the product therefore did not contain cannabinoid acid(s), such as CBDa or THCa.

TABLE 6

| | Cycling of Decarboxylated Crude (Cannabinoid Percentage) | | | | | | | | |
|---|---|---|---|---|---|---|---|---|---|
| | Reduced | Non-Reduced | | | | | | | Reduced |
| | (ethanol | (includes solvent) | | | | | | | (ethanol |
| | removed) | | Cycle | Cycle | Cycle | Cycle | Cycle | | removed) |
| | Crude | Matrix | 1 | 2 | 3 | 4 | 5 | Rinse | Eluent |
| Total | 65.3 | 1.8 | 1.5 | 1.3 | 1.3 | 1.2 | 1.1 | 0 | 74.37 |
| THC | 2.58 | 0.08 | 0.07 | 0 | 0.06 | 0.06 | 0 | 0 | 2.51 |
| CBD | 61.09 | 1.69 | 1.46 | 1.32 | 1.23 | 1.19 | 1.11 | 0.03 | 69.29 |
| CBG | 1.66 | 0 | 0 | 0 | 0 | 0 | 0 | 0 | 2.57 |

### 3.2 Cycling of Carboxylated Extract

The general parameters of the experiment are shown in Table 7.

TABLE 7

| | |
|---|---|
| Column | EGDMA/TBA (catechin template) Beads (0.5-1.5 mm |
| | diameter), 72.45 g |
| Flow rate | 45 mL/min |
| Extract | 1 g of Carboxylated winterized extract, 250 mL of |
| Solution | Ethanol, and 250 mL of H₂O Sonicated to form a solution. |
| | (~0.2 g of extract per 100 mL) |
| Rinse | 160 mL of H₂O and 40 mL of Ethanol (20% ethanol |
| Solution | Solution) |
| Elution | 500 mL of Ethanol |

Step 1: 500 mL of the Extract solution was pumped through the column and collected. Step 2: a sample of the extract solution was taken for analysis. Steps 1-2 were repeated 4 more times, i.e. the same extract solution was repeatedly put through the column to increase cannabinoid capture by the MIP. 200 mL of the 20%˜ ethanol rinse was then pumped through the Column. 500 mL of ethanol eluent was pumped through the column. The extract solution and eluent were reduced using a rotary evaporator. The samples of extract and the eluent concentrate were taken and sent for analysis.

The results for the carboxylated extract are shown in Table 8 and FIG. 4. The “matrix” in Table 6 is the emulsion/solution of crude Cannabis extract. In particular, the results show the MIP column is able to bind/extract a cannabinoid in acid form, in this case CBDA. The results show the proportion of CBD, CBG and THC increased.

TABLE 8
Carboxylated Crude (Cannabinoid Percentage)

| | Crude | Matrix | Eluent |
|---|---|---|---|
| Total | 66.41 | 5.32 | 59.16 |
| THC | 13.30 | 0.12 | 14.35 |
| CBD | 20.97 | 0.05 | 21.15 |
| CBG | 1.85 | 0.00 | 2.11 |
| CBDA | 30.30 | 5.16 | 21.55 |

### 4. Terpene Extraction Using MIPS

The general parameters of the experiment are shown in Table 9.

TABLE 9

| | |
|---|---|
| Column 1 | EGDMA/TBA (catechin template) Beads, 233.45 g, |
| | approx.. 0.5-2 mm diameter |
| Column 2 | DVB/Styrene (catechin template) Beads, 223.02 g, |
| | approx.. 0.5-1.5 mm diameter |
| Flow rate | 100 mL/min |
| (both columns) | |
| Extract Solution | 50 g of decarboxylated non-winterized extract, 1 L of |
| (both columns) | Ethanol, and 1 L of H₂O Sonicated to form a solution. |
| | (~2.5 g of extract per 100 mL) |
| Rinse Solution | 1.6 L of H₂O and 0.4 L of Ethanol (20% ethanol |
| (both columns) | Solution) |
| Elution (both | 2 L of Ethanol |
| columns) | |

2 L of the extract solution was pumped through the column 1. 2 L of the 20% ethanol rinse was then pumped through the column 1. The rinse was collected for later reduction and sampling. 2 L of ethanol eluent was pumped through the column 1.

The all of the samples were reduced using a rotary evaporator. The rinse samples were collated for evaporation (concentration). Once fully evaporated, vacuum and heat were applied to remove any remaining ethanol. The samples concentrates were taken and sent for terpene analysis.

This procedure was repeated with column 2.

Cannabinoids were collected but not quantified in this experiment. The results for the terpenes are shown in Tables 10 and 11. The results show that the terpene content of the cannabinoid extract produced by MIPs can be tailored through the choice of MIP recipe. The Column 1 MIP reduces the terpene content and the Column 2 MIP increases the terpene content and this can be done without significantly affecting the cannabinoid content. Decarboxylated crude extracts are generally known to have lower amounts of terpenes, but the results show even at low levels of some of the terpenes are preserved or at least not largely decreased by the MIP purification.

TABLE 10
Column 1

| | Reference | |
|---|---|---|
| Terpenes (wt %) | (crude cannabis extract) | Eluent |
| Linalool | 0.105 | 0.033 |
| caryophyllene oxide | 0.039 | 0.034 |
| guaiol | 0.051 | 0.044 |
| alpha-bisabolol | 0.070 | 0.07 |
| beta-caryophyllene | 0.460 | 0.175 |
| delta-3-carene | — | — |
| beta-myrcene | 0.025 | — |
| D-limonene | 0.012 | — |
| alpha-humulene | 0.157 | 0.079 |
| trans-nerolidol | 0.010 | 0.01 |
| Total | 0.900 | 0.4 |

TABLE 11
Column 2

| | Reference | |
|---|---|---|
| Terpenes (wt %) | (crude cannabis extract) | Eluent |
| Linalool | 0.105 | 0.145 |
| caryophyllene oxide | 0.039 | 0.059 |
| guaiol | 0.051 | 0.074 |
| alpha-bisabolol | 0.070 | 0.113 |
| beta-caryophyllene | 0.460 | 0.577 |
| delta-3-carene | — | — |
| beta-myrcene | 0.025 | — |
| D-limonene | 0.012 | — |
| alpha-humulene | 0.157 | 0.226 |
| trans-nerolidol | 0.010 | 0.02 |
| Total | 0.900 | 1.2 |

### 5. Separation of Cannabinoids

The general parameters of the experiment are shown in Table 12.

TABLE 12

| | |
|---|---|
| Column | EGDMA/TBA (catechin template) Beads, 233.45 g |
| Flow rate | 100 mL/min |
| (both columns) | |
| Extract | 50 g of decarboxylated Extract, 1 L of Ethanol, and 1 L |
| Solution | of H₂O Sonicated to form a solution. (~2.5 g of extract |
| | per 100 mL) |
| Rinse | 1.6 L of H₂O and 0.4 L of Ethanol (20% ethanol |
| Solution | Solution) |
| Elution | 2 L of Ethanol |

2 L of the crude extract solution was pumped through the column. 2 L of the 20% ethanol rinse was then pumped through the Column. 2 L of Ethanol Eluent was then pumped through the column; the eluent was collected in 200 mL volume fractions.

The 3 steps above were repeated 3 more times so that a total volume of 8 L of extract, rinse and eluent had been passed over the MIP beads. The eluents were collated from each pass to increase the final yield of extract. The eluent was reduced using a rotary evaporator, then vacuum and heat applied to remove any remaining ethanol. Samples of the eluent fraction concentrates were taken and sent for analysis.

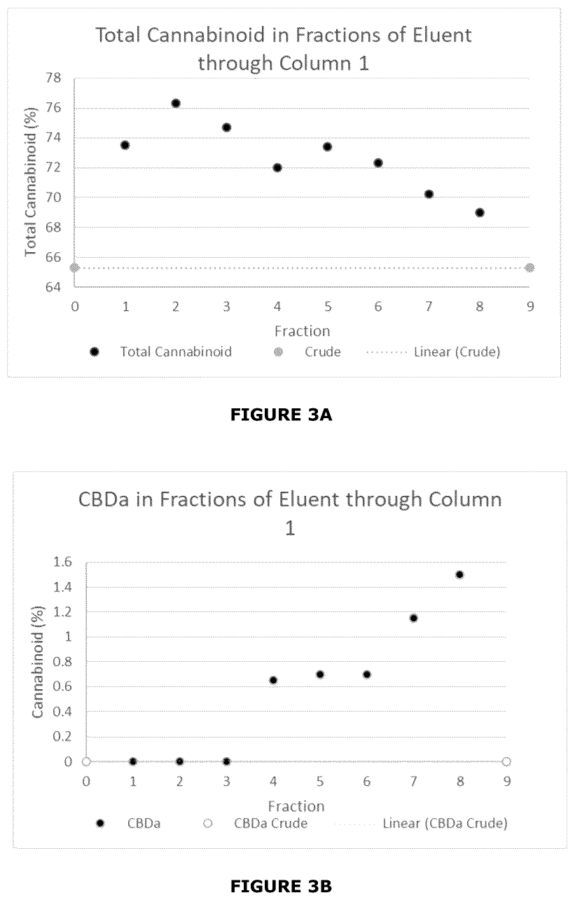

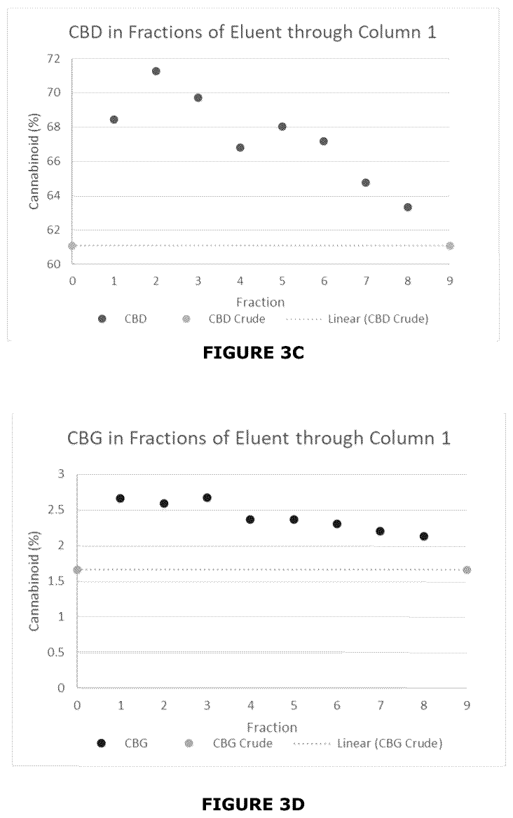

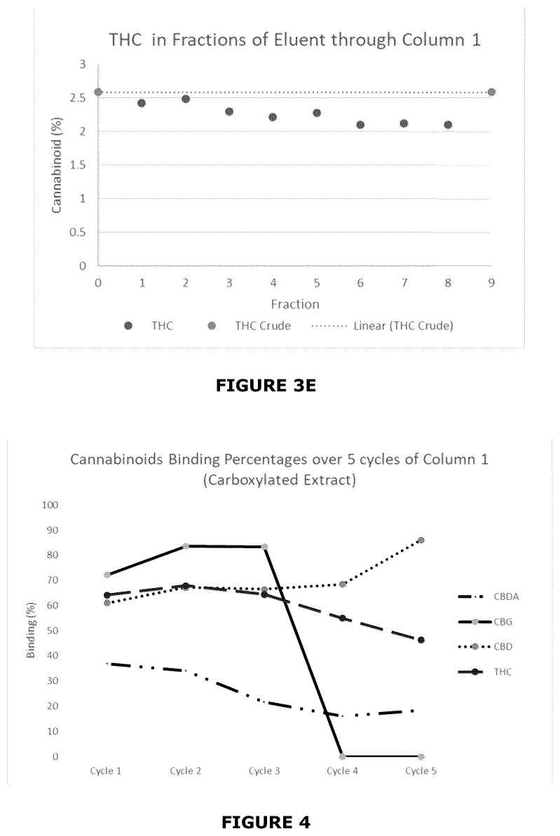

The results are shown in Table 13 and FIGS. 3A, 3B, 3C, 3D and 3E. The results show that there are changes in the cannabinoid profile in different eluent fractions. Particularly interesting is the appearance of CBDA in the latter eluent fractions. This indicates the method can be used to at least partially separate cannabinoids to produce compositions with higher levels of selected cannabinoids.

BDL indicates below detectable levels.

TABLE 13

| | Reference | | | | | | | | |
|---|---|---|---|---|---|---|---|---|---|
| | Material | Average Fraction | | | | | | | |
| | 0 | Frac 1 | Frac 2 | Frac 3 | Frac 4 | Frac 5 | Frac 6 | Frac 7 | Frac 8 |
| Total | 65.3 | 73.5 | 76.3 | 74.7 | 72 | 73.4 | 72.3 | 70.2 | 69 |
| THC | 2.58 | 2.41 | 2.48 | 2.29 | 2.21 | 2.27 | 2.09 | 2.12 | 2.09 |
| CBDA | BDL | 0 | 0 | 0 | 0.65 | 0.7 | 0.7 | 1.15 | 1.5 |
| CBD | 61.09 | 68.45 | 71.26 | 69.71 | 66.79 | 68.04 | 67.17 | 64.74 | 63.33 |
| CBG | 1.66 | 2.66 | 2.59 | 2.67 | 2.36 | 2.36 | 2.3 | 2.2 | 2.13 |

### 6. Binding and Elution of Terpenes

An experiment was conducted to demonstrate the binding and elution of terpenes by the imprinted polymers. A hemp extract was used that was not winterized or decarboxylated. The general parameters of the experiment are shown in Table 14.

TABLE 14

| | |
|---|---|
| Column 1 | EGDMA/TBA (catechin template) Beads, 63 g, |
| | approx.. 0.5-2 mm diameter |
| Column 2 | DVB/Styrene (catechin template) Beads, 61.58 g, |
| | approx.. 0.5-1.5 mm diameter |
| 1st Flow rate | 15 mL/min |
| (both columns) | |
| 2nd Flow rate | 50 mL/min |
| (both columns) | |
| Extract Solution | 1.25 g hemp extract, 250 mL 100% Ethanol spiked |
| (both columns) | with 4-Isopropyltoluene (p-Cymene), Alpha Bisabolol, |
| | Alpha Humulene, Beta-Caryophyllene, Alpha-Pinene, |
| | Beta-Myrcene, Beta-Pinene, Delta-Limonene, |
| | Gamma-Terpinene, Guaiol and Linalool. 250 mL H₂O |
| Rinse Solution | 100 mL 100% Ethanol and 400 mL of H₂O |
| (both columns) | |
| Elution (both | 500 mL 100% Ethanol |
| columns) | |

The method used was as follows:
Step 1: A 5 mL sample of the extract solution was taken for analysis.
Step 2: The extract solution was pumped through column 1 at 15 mL/min.
Step 3: A 5 mL sample was taken for analysis.
Step 4: The Rinse solution was pumped through the column at 50 mL/min.
Step 5: A 5 mL sample was taken for analysis.
Step 6: The Eluent solution was pumped through the column at 15 mL/min.
Step 7: A 5 mL sample was take for analysis.
Steps 1-7 were repeated using column 2.

The results are shown in Tables 15 and 16.

The initial terpene content was measured in the matrix (extract solution) before pumping through the MIP column. Then the final terpene content is measured in the matrix (extract solution) after pumping through the column MIP. The change in terpene content of the two solutions is used to calculate the “Bound” percentage in the Tables. The rinse solution was passed through the MIP column to flush off any fouling. Then the eluent is used to elute the bound components from the MIP. The terpene content of the eluent is measured. The terpene content in the eluent is compared to the amount of terpenes that bound to calculate the percentage of the terpenes that were able to be eluted to give the “Eluted” percentage in the Tables.

TABLE 15
Column 1 - average terpene binding and elution percentages

| Terpenes | Bound | Eluted |
|---|---|---|
| 4-Isopropyltoluene (p-Cymene), | 71.04 | 99.91 |
| alpha Bisabolol, | 52.64 | 114.27 |
| alpha Humulene, | 53.80 | 95.02 |
| alpha-Pinene, | 70.18 | 83.96 |
| beta-Caryophyllene, | 53.81 | 92.52 |
| beta-Myrcene,. | 70.18 | 81.56 |
| beta-Pinene, | 66.03 | 86.01 |
| delta-Limonene, | 70.17 | 89.87 |
| gamma-Terpinene, | 68.11 | 103.75 |
| Guaiol | 40.09 | 64.19 |
| Linalool | 47.33 | 93.14 |

TABLE 16
Column 2 - average terpene binding and elution percentages

| Terpenes | Bound | Eluted |
|---|---|---|
| 4-Isopropyltoluene (p-Cymene), | 81.13 | 105.96 |
| alpha Bisabolol, | 75.78 | 73.07 |
| alpha Humulene, | 77.97 | 65.34 |
| alpha-Pinene, | 70.01 | 96.18 |
| beta-Caryophyllene, | 73.52 | 74.55 |
| beta-Myrcene,. | 71.78 | 88.30 |
| beta-Pinene, | 86.17 | 80.54 |
| delta-Limonene, | 93.33 | 80.87 |
| gamma-Terpinene, | 92.19 | 102.33 |
| Guaiol | 68.92 | 66.35 |
| Linalool | 69.35 | 59.84 |

The results show that both MIP columns are binding and eluting terpenes from the extract. However, the MIP column 2 (DVB/Styrene beads) is more effective at both binding and eluting the majority of the terpenes tested than the MIP column 1 (EGDMA/TBA beads).

### 7. Heavy Metal Remediation

An experiment was conducted to demonstrate the ability of the MIPs to reduce heavy metals in crude extracts (hemp extract).

Seven metals were selected based on their prevalence as a contaminant in Cannabis and hemp products. The metals were arsenic, cadmium, chromium, copper, lead, nickel and zinc. A hemp extract was spiked with these metals (between 2-17 ppm) and treated with both the EGDMA/TBA and DVB/Styrene MIPs. Three treatments were carried out on each MIP with no cleaning step carried out in between. The general parameters of the experiment are shown in Table 17.

TABLE 17

| Parameter | Details |
|---|---|
| Column 1 | EGDMA/TBA (catechin template)Beads 17.73 g |
| Column 2 | DVB/Styrene (catechin template)Beads 15.52 g |
| Flow Rate | 5 mL/min |
| (both columns) | |
| Extract Solution | 1 g of Extract (not decarboxylated), 42.5 ml of |
| (both columns) | Ethanol, 42.5 mL of H2O spiked with Cadmium, |
| | Lead, Copper, Chromium, Nickle and Zinc, and |
| | Arsenic. |
| Rinse Solution | 17 mL of Ethanol and 68 mL of H2O |
| (both columns) | |
| Elution Solution | 85 mL of Ethanol |
| (both columns) | |

The method used was as follows:
Step 1: 85 mL of the heavy metal spiked Extract solution was pumped through the column at 5 mL/min.
Step 2: a sample of the extract solution was taken for analysis.
Step 3: 85 mL of the 20% ethanol rinse was then pumped through the column at 5 mL/min. Step 4: a sample of the rinse solution was taken for analysis.
Step 5: 85 mL of ethanol eluent was pumped through the column at 5 mL/min.
Step 6: a sample of the eluent solution was taken for analysis.
Steps 1-6 were repeated twice more.

The experiment was repeated on column 2.

The averaged results of the testing are shown in Tables 18 and 19.

TABLE 18
Average Column 1 (EGDMA/TBA) Percentage Heavy Metal Remediation

| | Arsenic | Cadmium | Chromium | Copper | Lead | Nickel | Zinc |
|---|---|---|---|---|---|---|---|
| Bound | 48.19 | 47.96 | 50.73 | 18.81 | 78.83 | 30.25 | 36.76 |
| Rinsed | 37.78 | 39.52 | 37.22 | 138.90 | 14.67 | 75.44 | 57.00 |
| Eluted | 30.54 | 22.54 | 37.43 | −42.00 | 19.95 | 26.18 | 49.41 |
| Total | 90.84 | 93.46 | 88.08 | 96.93 | 86.58 | 98.05 | 92.19 |
| Reduction | | | | | | | |

TABLE 19
Average Column 2 (DVB/Styrene) Percentage Heavy Metal Remediation

| | Arsenic | Cadmium | Chromium | Copper | Lead | Nickel | Zinc |
|---|---|---|---|---|---|---|---|
| Bound | 48.20 | 50.81 | 55.52 | 5.99 | 92.34 | 27.97 | 33.55 |
| Rinsed | 30.96 | 28.15 | 29.37 | 397.45 | 5.11 | 67.15 | 51.33 |
| Eluted | 9.96 | 9.73 | 9.82 | −22.76 | 6.09 | 13.08 | 16.70 |
| Total | 96.69 | 96.45 | 96.15 | 95.94 | 94.66 | 98.80 | 97.27 |
| Reduction | | | | | | | |

The results show that both MIPs showed a significant reduction in all of the metals contaminating the extract. However, the DVB/Styrene MIP is more effective than the EGDMA/TBA MIP.

### 8. Pesticide Remediation

An experiment was conducted to demonstrate the ability of MIPs to reduce pesticides in crude extracts (hemp extract).

Six pesticides were selected based on their prevalence as a contaminant in Cannabis and hemp products. The pesticides selected were Myclobutanil, Pyrimentanil, Carbaryl, Permethrin, Diazinon and Ethoprophos. A hemp extract was spiked with these pesticides at 5 ppm and treated with both EGDMA/TBA MIP beads and DVB/Styrene MIP Beads. The general parameters of the experiment are shown in Table 20.

TABLE 20

| Parameter | Details |
|---|---|
| Column 1 | EGDMA/TBA (catechin template) Beads 18.24 g |
| Column 2 | DVB/Styrene (catechin template) Beads 17.77 g |
| Flow Rate | 5 mL/min |
| (both columns) | |
| Extract Solution | 1 g of Extract (not decarboxylated), 42.5 mL of |
| (both columns) | Ethanol 10 ppm Myclobutanil, Pyrimentanil, Carbaryl, |
| | Permethrin, Diazinon and Ethoprophos, 42.5 mL of |
| | H₂O. |
| Rinse Solution | 17 mL of Ethanol and 68 mL of H₂O |
| (both columns) | |
| Elution Solution | 85 mL of Ethanol |
| (both columns) | |

The method used was as follows:
Step 1: 85 mL of the pesticide spiked Extract solution was pumped through the column at 5 mL/min.
Step 2: a sample of the extract solution was taken for analysis.
Step 3: 85 mL of the 20 ethanol rinse was then pumped through the column at 5 mL/min.
Step 4: a sample of the rinse solution was taken for analysis.
Step 5: 85 mL of ethanol eluent was pumped through the column at 5 mL/min.
Step 6: a sample of the eluent solution was taken for analysis.

This procedure was carried out on both columns of MIPs

The results are shown in Tables 21 and 22.

TABLE 21
Average percentage pesticide remediation Column 1 (EGDMA/TBA Beads)

| | Myclobutanil | Pyrimethanil | Carbaryl | Permethrin | Diazinon | Ethoprophos |
|---|---|---|---|---|---|---|
| Bound | 54.885 | 129.615 | 67.151 | 79.038 | 71.795 | 58.537 |
| Rinsed | 19.001 | 1.777 | 4.762 | 7.826 | 6.122 | 12.500 |
| Eluted | 78.281 | 41.211 | 61.905 | 70.435 | 73.469 | 68.750 |
| Total | 57.036 | 46.584 | 58.430 | 44.330 | 47.253 | 59.756 |
| Reduction | | | | | | |

TABLE 22
Average percentage pesticide remediation Column 2 (DVB/Styrene Beads)

| | Myclobutanil | Pyrimethanil | Carbaryl | Permethrin | Diazinon | Ethoprophos |
|---|---|---|---|---|---|---|
| Bound | 77.293 | 45.637 | 67.552 | 82.155 | 80.292 | 88.889 |
| Rinsed | 19.546 | 0.753 | 3.057 | 7.377 | 5.455 | 8.333 |
| Eluted | 40.506 | 62.927 | 54.585 | 27.049 | 56.818 | 52.778 |
| Total | 68.692 | 71.282 | 63.127 | 77.778 | 54.380 | 53.086 |
| Reduction | | | | | | |

The results show that both MIPs are showing a significant reduction in all of the pesticides contaminating the extract. The DVB/Styrene MIP is more effective than the EGDMA/TBA MIP.

### 9. Removal of Fats and Waxes

An experiment was conducted to demonstrate the removal of fats and waxes from an extract (hemp extract) using a MIP.

Winterization is a common process in the Cannabis and hemp industry. It is used to remove plant fat and waxes from the extract after the extraction step and is a bottle neck in the extraction processing. Winterization involves dissolving an extract in solvent (to form a winterizing solution), then chilling to solidify the fats/waxes/other lipids, which can then be filtered off.

The general parameters of the experiment are shown in Table 23.

TABLE 23

| Parameter | Details |
|---|---|
| Column | EGDMA/TBA (catechin template) Beads |
| | 405 g |
| Extract Solution Flow Rate | 40 mL/min |
| Rinse Solution Flow Rate | 100 mL/min |
| Eluent Solution Flow Rate | 40 mL/min |
| Crude Extract Winterizing | 5 g Crude Extract, 160 mL 95% ethanol |
| Solution | |
| Extract Solution | 43 g Crude Extract, 800 mL Ethanol, |
| | 800 mL H₂O |
| Rinse Solution | 320 mL of Ethanol and 1.28 L of H2O |
| Eluent Solution | 1.6 L Ethanol |
| Eluent Winterizing Solution | 2 g Eluent extract, 20 mL 95% ethanol |
| Filter Paper | Whatman No1 Filter Paper |

For the testing, the crude extract and an eluent which had been exposed to a EGDMA/TBA MIP made according to the methods described herein were winterized and filtered to determine the difference in the fat and wax content.

The method used was as follows:
Step 1: The extract solution was pumped through the column of MIP beads.
Step 2: The rinse solution was then pumped through the column of MIP beads.
Step 3: The eluent solution was pumped through the column of MIP beads.
Step 4 The eluent solvent was evaporated/concentrated (rotovaped) and the eluent extract recovered.
Step 5: The recovered eluent extract was dissolved in 95% ethanol to make the Eluent Winterizing Solution.
Step 6: The Eluent Winterizing Solution was placed in a freezer with dry ice for 24 hours.
Step 7: The Crude Extract Winterizing Solution was also placed in a freezer with dry ice for 24 hours.
Step 8: The Crude Extract Winterizing Solution and Eluent Winterizing Solutions were vacuum filtered using a buchner funnel and filter paper.
Step 9: The filter papers were allowed to dry and the weights then taken.

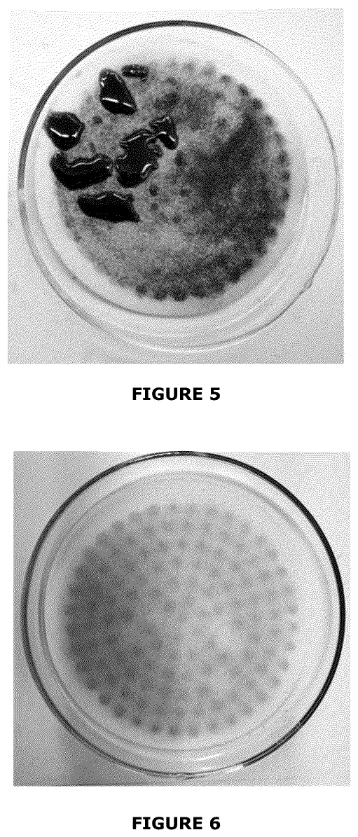

The averaged results of this testing are given in Table 24 and are shown in FIGS. 5 and 6. FIG. 5 shows the crude extract filter paper after winterization and filtration. FIG. 6 shows the eluent filter paper after winterization and filtration. The percentage fat/wax and other particulate matter given in the table were calculated from the mass of the filter papers before filtration and after filtration of the winterised solutions to collect the fats and waxes and other particulate matter (after drying to remove ethanol and water content). The masses of the fats/waxes/particulate matter collected were compared to the mass of the crude extract used to give the percentage.

TABLE 24

| | Fat/Wax/particulate matter content (%) |
|---|---|
| Crude Extract | 17.06 |
| Eluent | 3.94 |
| Total reduction | 76.93 |

There is a clear reduction in the fat/wax/particulate matter content of the extract after being passed through the column of MIP beads (i.e. exposed to the MIP). This is shown by the percentage reduction in fat/wax/other particulate matter content in Table 24 and the appearance of the filter papers in FIGS. 5 and 6. The appearance of the filter papers before and after treatment (i.e. filter paper with very dark coloured globules compared to light coloured filter paper without noticeable collection of matter) indicates various undesirable fats, waxes, and coloured compounds are removed by treatment with the MIP. The dark colour and consistency of the Crude Extract Winterizing Solution compared to the much clearer and less coloured eluent following exposure to MIP and Eluent Winterizing Solution also indicates removal of various fats/waxes/coloured compounds and other undesirable components in the crude extract. Based on the visual appearance of the filter paper in FIG. 6 (eluent), it is believed that the fat/wax proportion of the 3.94% content remaining on the filter paper is minimal.

### 10. Binding and Elution Testing of MIPs

An experiment was conducted to demonstrate the binding and elution various of MIPs.

The experiments were carried out using hemp extract that was not winterized or decarboxylated. The general parameters of the experiment are shown in Table 25.

TABLE 25

| Parameter | Details |
|---|---|
| MIP 1 | Catechin template, Acrylic acid/EGDMA Beads 10.0 g. |
| MIP 2 | CBD template, EGDMA Beads (EGDMA only) 10.03 g. |
| MIP 3 | CBD template, Methacrylic Acid/EDGMA 10.0 g |
| Extract | 1.25 g Extract, 50 mL Ethanol, 50 mL H₂O |
| Solution | |
| Eluent | 100 mL Ethanol |
| Solution | |

The method used was as follows:
Step 1: The MIP was placed in a beaker on a stirring plate.
Step 2: Extract solution was added to the beaker and stirred for 120 min.
Step 3: Samples were taken every 15 min for the first 60 min and then every 30 min for the final 60 min.
Step 4: The remaining solution was poured off and the MIP allowed to dry overnight.
Step 5: The eluent solution was added to the MIP beaker and stirred on a stirring plate for 120 min.
Step 6: Samples were taken every 15 min for the first 60 min and then every 30 min for the final 60 min.
Steps 1-6 were repeated for each MIP (MIPs 1, 2 and 3).

The results are shown in Tables 26, 27 and 28.

TABLE 26
MIP 1 (Catechin template, Acrylic acid/EGDMA
Beads) Percentage Binding and Elution

| | CBG | CBD | THC | CBDA | CBC | THCA | CBN |
|---|---|---|---|---|---|---|---|
| Bound | 65.00 | 63.00 | 67.00 | 51.00 | 67.00 | 66.00 | 67.00 |
| Eluted | 112.00 | 86.00 | 105.00 | 130.00 | 113.00 | 109.00 | 114.00 |

TABLE 27
MIP 2 (CBD template, EGDMA Beads)
Percentage Binding and Elution

| | CBG | CBD | THC | CBDA | CBC | THCA |
|---|---|---|---|---|---|---|
| Bound | 88.39 | 87.00 | 83.75 | 61.13 | 87.87 | 47.34 |
| Eluted | 100.34 | 70.62 | 91.28 | 47.16 | 79.78 | 41.33 |

TABLE 28
MIP 3 (CBD template, Methacrylic Acid/EDGMA)
Percentage Binding and Elution

| | CBG | CBD | THC | CBDA | CBC | THCA | CBN |
|---|---|---|---|---|---|---|---|
| Bound | 60.61 | 54.69 | 64.93 | 32.35 | 69.32 | 47.81 | 65.93 |
| Eluted | 75.13 | 84.80 | 79.70 | 13.85 | 37.18 | 12.40 | 14.86 |

The results showed that MIP 1 (catechin template, acrylic acid/EGDMA) and MIP 3 (CBD template, methacrylic Acid/EDGMA) had binding percentages below 70%. MIP 2 (CBD template, EGDMA) mainly had binding percentage over 80%, but had lower affinity for the acidic cannabinoids.

MIP 1 appears to have a higher affinity for cannabinoid acids than MIPs 2 and 3, While the binding percentages for MIP 1 are below 70%, the elution percentages show complete elution of the bound cannabinoids. The high elution percentages could make this MIP useful for working with reduced eluent volumes. This may be beneficial at large scale for reducing solvent volumes. MIP 1 having a higher affinity for the acidic cannabinoids may be beneficial as inclusion of acidic cannabinoids in enriched cannabinoid extract is attractive to some consumers.

The MIP 2 has a higher binding percentage than both the MIP 1 and MIP 3 and the elution percentages are relatively high as well, with high elution rates for CBG and THC. The binding and elution of the acidic cannabinoids is not very high, with THCA being below 50% for both the binding and elution.

MIP 3 showed a lower binding percentage for all the cannabinoids, with the acidic cannabinoids being particularly low. The elution of CBG, CBD and THC were much higher than the rest of the cannabinoids, with the elution percentages of CBDA, THCA and CBN being below S MIP 3 may therefore be considered for use to target only CBG, CBD and THC over the other cannabinoids. As CBG, CBD and THC are some of the most sought after cannabinoids, particularly for isolates, this may be of benefit.

11. Binding and Elution of Cannabinoids, Remediation of Fats and Waxes, Heavy Metals and Pesticides

An experiment was conducted to demonstrate the binding and elution of cannabinoids, and remediation of fats and waxes, heavy metals and pesticides using the EGDMA MIP.

The experiments were carried out using hemp extract that was not winterized or decarboxylated. The general parameters of the experiment are shown in Table 29.

TABLE 29

| Parameter | Details |
|---|---|
| Column 1 | EGDMA Beads (CBD template) 20.865 g |
| Flow Rate | 5 mL/min |
| (Extract and | |
| Eluent Solutions) | |
| Flow Rate | 20 mL/min |
| (Rinse Solution) | |
| Extract Solution | 1.01 g Extract, 55 mL 100% Ethanol spiked with |
| | 4-Isopropyltoluene (p-Cymene), alpha-Bisabolol, |
| | alpha-Humulene, beta-Caryophyllene, alpha-Pinene, |
| | beta-Myrcene, beta-Pinene, delta-Limonene, |
| | gamma-Terpinene, Guaiol, Linalool, Diazinon, |
| | Carbaryl, Permethrin, Myclobutanil and |
| | Pyrimethanil. 55 mL H2O Spiked with Cadmium, |
| | Lead, Copper, Chromium, Nickle and Zinc, and |
| | Arsenic. |
| Rinse Solution | 20 mL 100% Ethanol and 80 mL of H2O |
| Eluent Solution | 100 mL 100% Ethanol |
| Crude Extract | 5 g Crude Extract, 160 mL 95% ethanol |
| Winterizing | |
| Solution | |
| Eluent Winterizing | 0.826 g Eluent extract, 8.26 mL 95% ethanol |
| Solution | |
| Filter Paper | Whatman No1 Filter Paper |

The method used was as follows:
Step 1: A 10 mL sample of the extract solution was taken for analysis.
Step 2: The extract solution was pumped through the column at 5 mL/min.
Step 3: A 10 mL sample was taken for analysis.
Step 4: The Rinse solution was pumped through the column at 20 mL/min.
Step 5: A 10 mL sample was taken for analysis.
Step 6: The Eluent solution was pumped through the column at 5 mL/min.
Step 7: A 10 mL sample was take for analysis.
Step 8: The eluent was rotovaped and the eluent extract recovered.
Step 9: The crude extract and the eluent extract were individually dissolved in 95% ethanol to form the crude winterizing and eluent winterizing solutions. The solutions were placed in a freezer with dry ice for 24 hours.
Step 10: The crude extract winterizing solution and eluent winterizing solutions were vacuum filtered using a Buchner funnel and filter paper.
Step 11: The filter papers were allowed to dry, and the weights then taken.

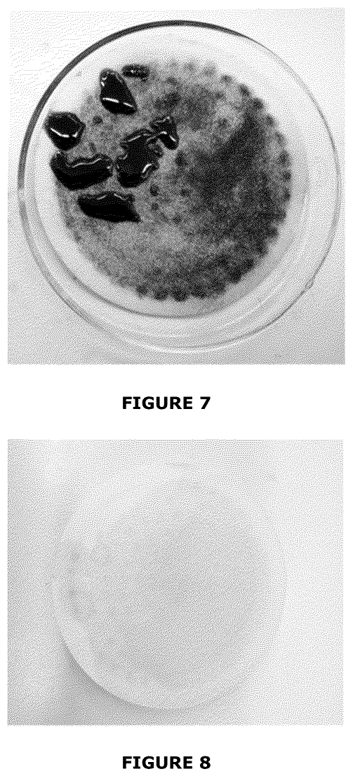

The results are shown in Tables 30 to 34 below and FIGS. 7 and 8. Table 30 shows the cannabinoid percentage in the extract solution and eluent. Table 31 shows the cannabinoid binding and elution percentages. Table 32 shows fat and waxes reduction percentages. Photos of the filter papers showing the difference in the fat and wax content in the crude extract and the eluent are also shown in FIGS. 7 and 8. Table 33 shows heavy metal remediation percentages. Table 34 shows the pesticide remediation percentages.

TABLE 30

| | Cannabinoid Percentages | | | | | | | |
|---|---|---|---|---|---|---|---|---|
| | CBG | CBD | THC | CBDA | CBC | THCA | Total | Difference |
| Crude | 0.61 | 40.17 | 0.87 | 14.74 | 0.45 | 0.23 | 57.07 | |
| Eluent | 0.46 | 37.82 | 0.89 | 10.82 | 0.36 | 0.21 | 50.56 | −6.51 |

TABLE 31
Percentage Cannabinoid Binding and Elution

| | CBG | CBD | THC | CBDA | CBC | THCA |
|---|---|---|---|---|---|---|
| Bound | 85.65 | 85.66 | 85.69 | 81.49 | 88.03 | 85.30 |
| Eluted | 91.81 | 77.23 | 70.38 | 68.53 | 88.48 | 78.65 |

TABLE 32
Fat and Wax content (%)

| | |
|---|---|
| Extract | 17.06 |
| Eluent | 3.27 |
| Total Reduction | 80.84 |

TABLE 33
Percentage Heavy Metal Remediation

| | Extract solution | Eluent solution | Total reduction |
|---|---|---|---|
| | (mg) | (mg) | (%) |
| Arsenic | 0.422 | 0.000 | 100 |
| Cadmium | 0.433 | 0.061 | 85.94 |
| Chromium | 0.177 | 0.010 | 94.41 |
| Copper | 0.271 | 0.022 | 91.82 |
| Lead | 0.102 | 0.008 | 92.44 |
| Nickel | 0.590 | 0.025 | 95.74 |
| Zinc | 0.470 | 0.000 | 100.00 |

TABLE 34
Percentage Pesticide Remediation

| | Extract Solution | Eluent solution | Total reduction |
|---|---|---|---|
| | (mg) | (mg) | (%) |
| Carbaryl | 0.935 | 0.392 | 58.07 |
| Diazinon | 0.021 | 0.017 | 17.94 |
| Myclobutanil | 0.611 | 0.343 | 43.82 |
| Permethrin | 0.025 | 0.069 | −177.17 |
| Pyrimethanil | 0.506 | 0.412 | 18.66 |

The results show that the MIP has high binding and elution rates for all the cannabinoids. The overall composition of the extract product in this example is not greatly altered, although there was a small increase in the THC content. The overall cannabinoid percentage in the extract after the MIP has dropped, by a small amount (6.5%).

Comparing this example (column treatment) to the batch treatment in Example 10, this example showed lower elution percentages for several of the cannabinoids. It is therefore believed the cannabinoid content of the eluent/enriched cannabinoid extract could be increased by use of a slower flow rate and/or more eluent solvent and/or different solvents. The MIPs used in this experiment were also new and unconditioned. It is believed the cannabinoid content could also be increased by conditioning the MIPs.

The results show an 80% decrease in fats and waxes. Heavy metal remediation percentages are very high for all the metals tested for. The pesticide remediation for this MIP is relatively low overall, there is some remediation of carbaryl and myclobutanil, but no remediation of permethrin.

The results for the terpenes showed unusually high percentages in the eluent and are therefore not believed to be reliable, so have not been provided here. This is believed to be due to a solubility issue with the doped terpenes.

### Representative Features

1. An imprinted polymer for producing an enriched cannabinoid extract from a crude Cannabis extract,
wherein the polymer is imprinted with a template organic molecule with a molecular weight of about 150 to 450 grams per mol comprising a hydroxyphenyl group, and
wherein the polymer has been prepared from one or more polymerizable monomers.

2. An imprinted polymer bead for producing an enriched cannabinoid extract from a crude Cannabis extract,
wherein the imprinted polymer bead is imprinted with a template organic molecule with a molecular weight of about 150 to 450 grams per mol comprising a hydroxyphenyl group, and
wherein the imprinted polymer bead has been prepared from one or more polymerizable monomer(s).

3. A method of making an imprinted polymer for producing an enriched cannabinoid extract from a crude Cannabis extract, the method comprising polymerizing one or more polymerizable monomer(s) in the presence of a template organic molecule with a molecular weight of about 150 to 450 grams per mol comprising a hydroxyphenyl group, and optionally subsequently at least partially removing the template molecule from the imprinted polymer.

4. A method of producing an enriched cannabinoid extract from a crude Cannabis extract, the method comprising the steps of:
a) contacting an imprinted polymer with the crude Cannabis extract, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers;
b) eluting the cannabinoids from the imprinted polymer with an elution solvent to produce the enriched cannabinoid extract comprising one or more cannabinoid(s).

5. The imprinted polymer, imprinted polymer bead or methods of any one of the preceding claims wherein the one or more polymerizable monomer are selected from an acryl or a vinyl, preferably the acryl monomer is selected from acrylic acid, tert-butylacrylamide, N-phenylacrylamide, N-methylacrylamide, methacrylic acid, 2-(hydroxyethyl)methacrylate, ethylene glycol dimethacrylate (EGDMA), methacrylic anhydride or trimethylolpropane trimethacrylate, optionally the acryl monomer is a methacryl monomer, preferably the methacryl monomer is selected from methacrylic acid, 2-(hydroxyethyl)methacrylate, ethylene glycol dimethacrylate, methacrylic anhydride or trimethylolpropane trimethacrylate, preferably the vinyl monomer is selected from a styryl or vinylpyridine, preferably the vinylpyridine is 4-vinylpyridine, preferably the styryl monomer is selected from styrene, 4-vinylstyrene.

6. The imprinted polymer, imprinted polymer bead or methods of any one of the preceding claims wherein the polymer has been prepared from one or more acryls (including methacryl) monomers, or the polymer has been prepared from one or more styryl monomers.

7. The imprinted polymer, imprinted polymer bead or methods of any one of the preceding claims wherein the polymer is prepared from two or more monomers, optionally at least one of the monomers acts as a crosslinker, preferably the crosslinker is a diolefin.

8. The imprinted polymer, imprinted polymer bead or methods of any one of the preceding claims wherein the polymer is prepared from two or more styryl monomers, optionally the polymer has been prepared from divinylbenzene (DVB) and styrene monomers, optionally the ratio of divinylbenzene (DVB) and styrene is about 1:0.01-0.5 by molar mass, preferably the ratio of divinylbenzene (DVB) and styrene is about 1:0.05-0.3 by molar mass, preferably the ratio of divinylbenzene (DVB) and styrene is about 1:0.1-0.3 by molar mass.

9. The imprinted polymer, imprinted polymer bead or methods of any one of the preceding claims wherein the polymer is prepared from two or more acryl (including methacryl) monomers, optionally the polymer has been prepared from ethylene glycol dimethacrylate (EGDMA) and methacrylic acid monomers, preferably the ratio of ethylene glycol dimethacrylate (EGDMA) and methacrylic acid is about 1:0.1-2 by molar mass, preferably the ratio of ethylene glycol dimethacrylate (EGDMA) and methacrylic acid is about 1:0.2-0.8 by molar mass, preferably the ratio of ethylene glycol dimethacrylate (EGDMA) and methacrylic acid is about 1:0.4-0.5 by molar mass.

10. The imprinted polymer, imprinted polymer bead or methods of any one of the preceding claims wherein the polymer has been prepared from acrylic acid and ethylene glycol dimethacrylate (EGDMA) monomers, optionally the ratio of acrylic acid and ethylene glycol dimethacrylate (EGDMA) is about 1:0.2-1.1 by molar mass, preferably the ratio of acrylic acid and ethylene glycol dimethacrylate (EGDMA) is about 1:0.4-0.8 by molar mass, preferably ratio of acrylic acid and ethylene glycol dimethacrylate (EGDMA) is about 1:0.6-0.7 by molar mass.

11. The imprinted polymer, imprinted polymer bead or methods of any one of the preceding claims wherein the polymer has been prepared from ethylene glycol dimethacrylate (EGDMA) and 2-(hydroxyethyl) methacrylate monomers, optionally the ratio of ethylene glycol dimethacrylate (EGDMA) and 2-(hydroxyethyl) methacrylate is about 1:1.5-0.05, preferably the ratio of ethylene glycol dimethacrylate (EGDMA) and 2-(hydroxyethyl) methacrylate is about 1:1.5-0.1 by molar mass, preferably the ratio of ethylene glycol dimethacrylate (EGDMA) and 2-(hydroxyethyl) methacrylate is about 1:1.2-0.8 by molar mass, preferably the ratio of ethylene glycol dimethacrylate (EGDMA) and 2-(hydroxyethyl) methacrylate is about 1:0.5-0.1 by molar mass.

12. The imprinted polymer, imprinted polymer bead or methods of any one of the preceding claims wherein the polymer has been prepared from ethylene glycol dimethacrylate (EGDMA) and tert-butylacryamide (TBA) monomers, optionally the ratio of ethylene glycol dimethacrylate (EGDMA) and tert-butylacryamide (TBA) is about 1:0.05-0.5 by molar mass, preferably the ratio of ethylene glycol dimethacrylate (EGDMA) and tert-butylacryamide (TBA) is about 1:0.05-0.3 by molar mass, preferably the ratio of ethylene glycol dimethacrylate (EGDMA) and tert-butylacryamide (TBA) is about 1:0.1-0.2 by molar mass.

13. The imprinted polymer, imprinted polymer bead or methods of any one of the preceding claims wherein the polymer has been prepared from styrene and ethylene glycol dimethacrylate (EGDMA) monomers, optionally the ratio of ethylene glycol dimethacrylate (EGDMA) and styrene and is about 1:0.1-1 by molar mass, preferably the ratio of ethylene glycol dimethacrylate (EGDMA) and styrene and is about 1:0.3-0.9 by molar mass, preferably the ratio of ethylene glycol dimethacrylate (EGDMA) and styrene and is about 1:0.4-0.8 by molar mass, preferably the ratio of ethylene glycol dimethacrylate (EGDMA) and styrene and is about 1:0.6-0.7 by molar mass.

14. The imprinted polymer, imprinted polymer bead or methods of any one of the preceding claims wherein the polymer has been prepared from ethylene glycol dimethacrylate (EGDMA) as the only monomer.

15. The imprinted polymer, imprinted polymer bead or methods of any one of the preceding claims wherein the imprinted polymer is in the form of a bead, preferably the bead is about 0.1 to 10 mm in diameter, preferably the bead is about 0.2 to 8 mm in diameter, preferably the bead is about 0.2 to 6 mm in diameter, preferably the bead is about 0.2 to 5 mm in diameter, preferably the bead is about 0.2 to 6 mm in diameter, preferably the bead is about 0.2 to 4 mm in diameter, preferably the bead is about 0.2 to 3 mm in diameter, preferably the bead is about 0.2 to 2 mm in diameter, preferably the bead is about 0.5-2 mm diameter, preferably the bead is about 0.5 to 1.5 mm in diameter, optionally about 99% of the beads are about 0.1 to 10 mm in diameter, preferably about 95% of the beads are about 0.1 to 10 mm in diameter, preferably about 90% of the beads are about 0.1 to 10 mm in diameter, preferably about 85% of the beads are about 0.1 to 10 mm in diameter, optionally the bead has compression strength of about 300-13,800 psi.

16. The imprinted polymer, imprinted polymer bead or methods of any one of the preceding claims wherein the template comprises the structure:

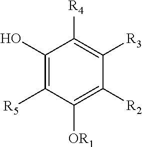

wherein R₁, R₂, R₃, R₄ and R₅ are the remainder of the organic molecule.

17. The imprinted polymer, imprinted polymer bead or methods of any one of the preceding claims wherein the template preferably comprises the structure:

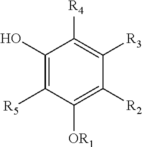

wherein R₁, R₂, R₃, R₄ and R₅ are the remainder of the organic molecule wherein
R₁ is H
R₂ is selected from H, or an organic group,
R₃ is selected from H, —OH, or an organic group,
R₄ is H or an organic group,
R₅ is H, or an organic group,
or R₁ and R₂ together form a 5- or 6-membered ring, preferably a 6-membered ring, optionally substituted with one or more of —OH, or an organic group, or
or R₁ and R₅ together form a 5- or 6-membered ring, preferably a 6-membered ring, optionally substituted with one or more substitutes independently selected from —OH, or an organic group, and wherein the 5- or 6-membered ring is optionally fused to a further ring which may be optionally substituted with one or more alkyl.

18. The imprinted polymer, imprinted polymer bead or methods of any one of the preceding claims wherein the template consists of C, H and O atoms.

19. The imprinted polymer, imprinted polymer bead or methods of any one of the preceding claims wherein one or more of the R₁, R₂, R₃, R₄ or R₅ groups is a saturated alkyl group, preferably a saturated alkyl group with 2 to 8 carbons.

20. The imprinted polymer, imprinted polymer bead or methods of any one of the preceding claims wherein at least one of R₂, R₃, and R₅ are an alkyl, preferably a 4-6 alkyl, or R₁ and R₂ or R₁ and R₅ together form the 5- or 6-membered ring.

21. The imprinted polymer, imprinted polymer bead or methods of any one of the preceding claims wherein R₃ is a C2-C8 saturated alkyl group, which may be branched or unbranched, preferably R₃ is an unbranched pentyl group.

22. The imprinted polymer, imprinted polymer bead or methods of any one of the preceding claims wherein R₂ and R₃ or R₃ and R₄ or R₁ and R₂ or R₁ and R₅ form a fused ring, preferably a 5- or 6-membered ring, preferably a 6-membered ring wherein the fused ring is optionally substituted, preferably the fused ring is substituted with a substituted or unsubstituted phenyl group, preferably the phenyl group is substituted with one or more alcohol groups, preferably the fused ring is substituted with an alcohol group.

23. The imprinted polymer, imprinted polymer bead or methods of any one of claims 16 to 22 wherein, R₁ is H
R₂ is selected from H, alkyl, or CORₓ, wherein Rₓ is alkyl, cycloalkyl or aryl, preferably aryl,
R₃ is selected from H, —OH or alkyl,
R₄ is H or alkyl,
R₅ is H, alkyl, or cycloalkyl optionally substituted with one or more alkyl or —OH,
or R₁ and R₂ together form a 5- or 6-membered ring, preferably a 6-membered ring, optionally substituted with one or more substituents independently selected from —OH, alkyl, or aryl, wherein the aryl is optionally substituted with one or more —OH,
or R₁ and R₅ together form a 5- or 6-membered ring, preferably a 6-membered ring, optionally substituted with one or more substituents independently selected from —OH, alkyl, or aryl, wherein the aryl is optionally substituted with one or more —OH, and wherein the 5- or 6-membered ring is optionally fused to a further 5- or 6-membered ring, preferably a cycloalkyl ring, which may be optionally substituted with one or more independently selected alkyl.

24. The imprinted polymer, imprinted polymer bead or methods of any one of claims 16 to 23 wherein, wherein R₂ is alkyl; and R₁, R₃, R₄, and R₅ are H, or wherein R₃ is alkyl; and R₁, R₂, R₄, and R₅ are H.

25. The imprinted polymer, imprinted polymer bead or methods of any one of claims 16 to 23 wherein R₂ is CORₓ wherein Rₓ is aryl, and R₁, R₃, R₄, and R₅ are H.

26. The imprinted polymer, imprinted polymer bead or methods of any one of claims 16 to 23 wherein, R₃ and R₅ are each independently alkyl; and R₁, R₂, and R₄ are H.
wherein R₃ is alkyl; R₅ is cycloalkyl optionally substituted with one or more alkyl groups, and R₁, R₂, and R₄ are H.

27. The imprinted polymer, imprinted polymer bead or methods of any one of claims 16 to 23 wherein, R₃ is OH; R₄ and R₅ are H; and R₁ and R₂ together form a 6-membered ring optionally substituted with one or more groups independently selected from —OH and phenyl, wherein the phenyl is optionally substituted with one or more —OH.

28. The imprinted polymer, imprinted polymer bead or methods of any one of claims 16 to 23 wherein, R₃ is alkyl; R₂ and R₄ are H; and R₁ and R₅ together form a 6-membered ring optionally substituted with one or more substituents independently selected from —OH, and alkyl, and wherein the 6-membered ring is optionally fused to a further 6-membered ring which may be optionally substituted with one or more independently selected alkyl.

29. The imprinted polymer, imprinted polymer bead or methods of any one of the preceding claims wherein the template is of the formula:

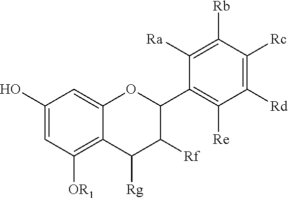

wherein Rₐ, R_{b}, R_{c}, R_{d}, Rₑ, R_{f} and R_{g} are optional substituents, preferably Rₐ, R_{b}, R_{c}, R_{d}, Rₑ are independently —OH or —H, preferably, R_{b}, R_{c} are —OH groups, preferably R_{b}, Rₑ are —OH groups and Rₐ, R_{d} and Rₑ are hydrogen groups.

30. The imprinted polymer, imprinted polymer bead or methods of any one of the preceding claims wherein the template molecule is a cannabinoid.

31. The imprinted polymer, imprinted polymer bead or methods of any one of the preceding claims wherein the template molecule is selected from:

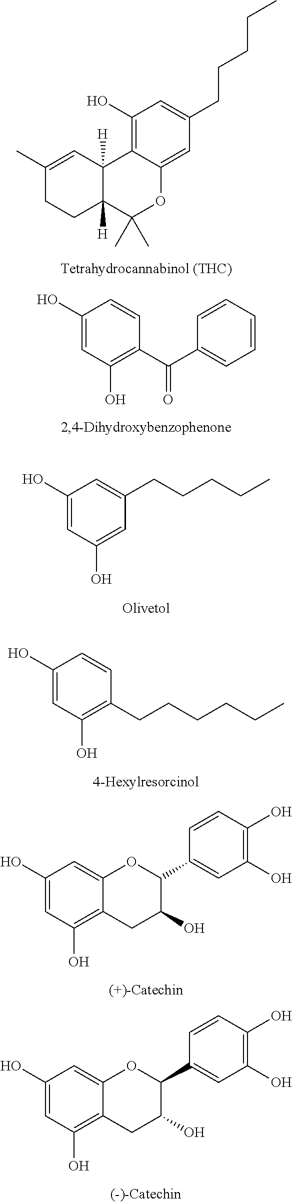

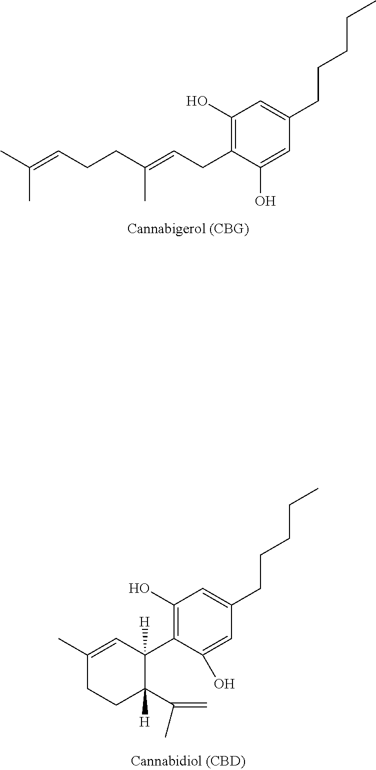

32. The imprinted polymer, imprinted polymer bead or methods of any one of the preceding claims wherein the template is selected from Cannabigerol (CBG), Cannabidiol (CBD), Tetrahydrocannabinol (THC).

33. The imprinted polymer, imprinted polymer bead or methods of any one of the preceding claims wherein the template is selected from:

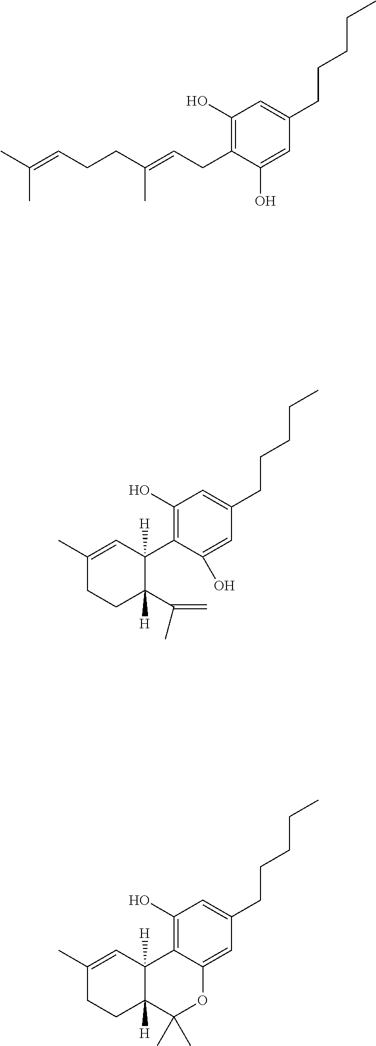

34. The imprinted polymer, imprinted polymer bead or methods of any one of the preceding claims wherein the template molecule is not a cannabinoid.

35. The imprinted polymer, imprinted polymer bead or methods of any one of the preceding claims wherein the template molecule is an alkyl- or acyl-resorcinol or an 2,4 Dihydroxybenzophenone or an olivetol or an 4-Hexylresorcinol or a flavonoid or flavan, preferably the template molecule is a flavan-3-ol.

36. The imprinted polymer, imprinted polymer bead or methods of any one of the preceding claims wherein the template molecule is catechin, preferably the template molecule is (+/−)-catechin.

37. The imprinted polymer, imprinted polymer bead or methods of any one of the preceding claims wherein the ratio of the template to the one or more monomer(s) is about 1:10-300 by molar mass, preferably the ratio of the template to the one or more monomer(s) is about 1:15-200 by molar mass, preferably the ratio of the template to the one or more monomer(s) is about 1:20-160 by molar mass, preferably the ratio of the template to the one or more monomer(s) is about 1:50-150 by molar mass, preferably the ratio of the template to the one or more monomer(s) is about 1:70-120 by molar mass.

38. The imprinted polymer, imprinted polymer bead or methods of any one of the preceding claims wherein the method of making an imprinted polymer comprises polymerizing one or more monomers in the presence of an initiator, preferably the initiator is an oil-soluble azo initiator, preferably the initiator is selected from dimethyl 2,2′-azobis(2-methylpropionate), 2,2′-azobis(isobutyronitrile) (‘AIBN’), 2,2′-azobis(4-methoxy-2,4-dimethylvaleronitrile), 2,2′-azobis(2,4-dimethylvaleronitrile) or 2,2′-azobis (2-methylbutyronitrile), optionally the ratio of initiator to the one or more monomer(s) is about 0.001-0.2:1 by molar mass, preferably the ratio of initiator to the one or more monomer(s) is about 0.01-0.1:1 by molar mass, preferably the ratio of initiator to the one or more monomer(s) is about 0.01-0.05:1 by molar mass.

39. The imprinted polymer, imprinted polymer bead or methods of any one of the preceding claims wherein polymerizing is carried out in a suspension liquid, preferably the suspension liquid is selected from one or more of water and organic solvents, such as mineral oil, perflurohydrocarbon, ethyl acetate, toluene, xylene, cyclohexane, hexane, benzene or heptane, preferably the suspension liquid is water.

40. The imprinted polymer, imprinted polymer bead or methods of any one of the preceding claims wherein a monomer pre-mix solution which is immiscible with the suspension liquid comprises the one or more monomer(s), template and initiator is provided, optionally the monomer pre-mix solution comprises the one or more monomer(s), template and initiator which are dissolved in at least one solvent, optionally the template are dissolved in the solvent prior to addition of the initiator, optionally the monomer pre-mix solution is added to the suspension liquid, optionally the solvent is selected from one or more of ethyl acetate, toluene, xylene, cyclohexane, hexane, benzene, heptane, acetonitrile, preferably the solvent is ethyl acetate or acetonitrile, preferably the solvent is acetonitrile and the suspension liquid is mineral oil, preferably the solvent is ethyl acetate and the suspension liquid is water.

41. The imprinted polymer, imprinted polymer bead or methods of any one of the preceding claims wherein the monomer pre-mix solution comprises acetonitrile, ethylene glycol dimethacrylate (EGDMA), the template and the initiator, optionally the monomer pre-mix solution comprises acetonitrile, methacrylic acid, ethylene glycol dimethacrylate (EGDMA), the template and the initiator, optionally the monomer pre-mix solution comprises acetonitrile, acrylic acid, ethylene glycol dimethacrylate (EGDMA), the template and the initiator, optionally the monomer pre-mix solution comprises acetonitrile, 2-(hydroxyethyl)methacrylate, ethylene glycol dimethacrylate (EGDMA), the template and the initiator, optionally the monomer pre-mix solution comprises ethyl acetate, divinylbenzene (DVB), styrene, the template and the initiator, optionally the monomer pre-mix solution comprises ethyl acetate, tert-butylacryamide (TBA), ethylene glycol dimethacrylate (EGDMA), the template and the initiator, optionally the monomer pre-mix solution comprises ethyl acetate, styrene, ethylene glycol dimethacrylate (EGDMA), the template and the initiator.

42. The imprinted polymer, imprinted polymer bead or methods of any one of the preceding claims wherein the ratio of suspension liquid and monomer pre-mix solution is about 0.5-80:1 by volume, preferably the ratio of suspension liquid and monomer pre-mix solution is about 0.5-50:1 by volume, preferably the ratio of water as the suspension liquid and monomer pre-mix solution comprising ethyl acetate is about 100-10:1 by volume, preferably the ratio of water as the suspension liquid and monomer pre-mix solution comprising ethyl acetate is about 20-60:1 by volume, preferably the ratio of water as the suspension liquid and monomer pre-mix solution comprising ethyl acetate is about 30-50:1 by volume, preferably the ratio of mineral oil as the suspension liquid and monomer pre-mix solution comprising acetonitrile is about 0.5-20:1 by volume, preferably the ratio of mineral oil as the suspension liquid and monomer pre-mix solution comprising acetonitrile is about 0.5-10:1 by volume, preferably the ratio of mineral oil as the suspension liquid and monomer pre-mix solution comprising acetonitrile is about 0.6-8:1 by volume.

43. The imprinted polymer, imprinted polymer bead or methods of any one of the preceding claims wherein the liquid is agitated, such that the polymer forms in beads, preferably the liquid is agitated at about 100-1500 RPM, preferably the liquid is agitated at about 200-1000 RPM, optionally the liquid is agitated for at least about 8 hours, preferably the liquid is agitated for at least about 10 hours, preferably the liquid is agitated for at least about 12 hours, preferably the liquid is agitated for about 10-24 hours.

44. The imprinted polymer, imprinted polymer bead or methods of any one of the preceding claims wherein the polymerization is maintained at a temperature of between about 40-80° C., preferably the polymerization is maintained at a temperature of between about 50-70° C.

45. The imprinted polymer, imprinted polymer bead or methods of any one of the preceding claims wherein the method of making an imprinted polymer comprises at least partially removing the template molecule from the imprinted polymer, preferably a solvent is used to at least partially remove the template molecule from the imprinted polymer.

46. The imprinted polymer, imprinted polymer bead or methods of any one of the preceding claims wherein the method of producing an enriched cannabinoid extract from a crude Cannabis extract the enriched cannabinoid extract has an increase of about 5-30% of total cannabinoids by mass.

47 The imprinted polymer, imprinted polymer bead or methods of any one of the preceding claims wherein the enriched cannabinoid extract has an increased proportion of CBD, or the enriched cannabinoid extract has an increased proportion of CBG, or the enriched cannabinoid extract has an increased proportion of CBD and CBG.

48. The imprinted polymer, imprinted polymer bead or methods of any one of the preceding claims wherein the enriched cannabinoid extract comprises a cannabinoid in acid form, preferably the cannabinoid in acid form is THCA and/or CBDA.

49. The imprinted polymer, imprinted polymer bead or methods of any one of the preceding claims wherein the enriched cannabinoid extract has a reduced proportion of heavy metals and/or pesticides.

50. The imprinted polymer, imprinted polymer bead or methods of any one of the preceding claims wherein the enriched cannabinoid extract has a reduced proportion of lipids, preferably the reduction in lipids is about 50%-100% by mass, preferably the reduction in lipids is about 60%-100% by mass, preferably the reduction in lipids is about 70%-100% by mass, preferably the reduction in lipids is about 80%-100% by mass, preferably the reduction in lipids is about 90%-100% by mass, preferably the reduction in lipids is about 95%-100% by mass, preferably the reduction in lipids is about 98%-100% by mass, preferably the reduction in lipids is about 99%-100% by mass, preferably the reduction in lipids is about 99.5%-100% by mass.

51. The imprinted polymer, imprinted polymer bead or methods of any one of the preceding claims wherein the enriched cannabinoid extract comprises less than 10% lipids, preferably the enriched cannabinoid extract comprises less than 5% lipids, preferably the enriched cannabinoid extract comprises less than 1% lipids, preferably the enriched cannabinoid extract comprises less than 0.05% lipids, preferably the enriched cannabinoid extract comprises less than 0.05% lipids, preferably the enriched cannabinoid extract comprises less than 0.01% lipids, preferably the enriched cannabinoid extract comprises substantially no lipids.

52. The imprinted polymer, imprinted polymer bead or methods of any one of the preceding claims wherein the enriched cannabinoid extract has an increased proportion of molecules within the size range 100 to 450 grams per mol, preferably more than 70%, preferably greater than 75%, preferably greater than 80%.

53. The imprinted polymer, imprinted polymer bead or methods of any one of the preceding claims wherein the enriched cannabinoid extract has a reduced proportion of non-cannabinoid molecules that cause a bitter taste.

54. The imprinted polymer, imprinted polymer bead or methods of any one of the preceding claims wherein the method of producing an enriched cannabinoid extract from a crude Cannabis extract the proportion of at least one terpene is increased, optionally the total proportion of terpenes is increased, optionally the proportion of at least one terpene is decreased, optionally the total proportion of terpenes is decreased, optionally the proportion of at least one terpene remains approximately the same, optionally the total proportion of terpenes remains approximately the same, preferably the terpene is selected from one or more of linalool, caryophyllene oxide, guaiol, alpha-bisabolol, beta-caryophyllene, beta-myrcene, D-limonene, alpha-humulene, trans-nerolidol, geraniol, valencene, terpineol, borneol, camphene, delta-3-carene, eucalyptol, alpha-pinene, beta-pinene, preferably the terpene that increases is selected from one or more of linalool, caryophyllene oxide, guaiol, alpha-bisabolol, beta-caryophyllene, alpha-humulene, trans-nerolidol, preferably the proportion of linalool, caryophyllene oxide, guaiol, alpha-bisabolol, beta-caryophyllene, alpha-humulene and trans-nerolidol increases, optionally the terpene that decreases is selected from one or more of beta-myrcene, linalool, guaiol, beta-caryophyllene, D-limonene, alpha-humulene, preferably the proportion of beta-myrcene, linalool, guaiol, beta-caryophyllene, D-limonene and alpha-humulene deceases, optionally the terpene that remains approximately the same is selected from one or more of caryophyllene oxide, alpha-bisabolol, trans-nerolidol preferably the proportion of caryophyllene oxide, alpha-bisabolol and trans-nerolidol remains approximately the same.

55. The imprinted polymer, imprinted polymer bead or methods of any one of the preceding claims wherein step (a) is followed by collection of the crude Cannabis extract that has been contacted with the imprinted polymer to give a Cannabis extract, optionally the Cannabis extract is used in step (a) in place of the crude Cannabis extract.

56. The imprinted polymer, imprinted polymer bead or methods of any one of the preceding claims wherein the method of producing an enriched cannabinoid extract from a crude Cannabis extract further comprises the step prior to step (a) of forming an emulsion and/or dissolving the crude Cannabis extract in one or more liquid(s) to give an emulsion and/or solution of crude Cannabis extract, such that the imprinted polymer is contacted with the crude Cannabis extract in the form of an emulsion and/or solution, preferably the emulsion and/or solution is substantially uniformly dispersed, optionally the emulsion and/or solution is prepared by sonication/ultrasonication and/or high shear mixing of the crude Cannabis extract and the one or more liquid(s), optionally the liquid(s) comprise one or more of water, ethanol, methanol, ethyl acetate, isopropyl alcohol, acetonitrile, acetone or THF, preferably the liquid(s) are selected from ethanol or water or a mixtures thereof, optionally the crude extract is dissolved/emulsified in about 5 to 100 ml of the one or more liquids per gram of the crude extract, preferably the crude extract is dissolved/emulsified in about 5 to 80 ml of the one or more liquids per gram of the crude extract, preferably the crude extract is dissolved/emulsified in about 5 to 60 ml of the one or more liquids per gram of the crude extract, preferably the crude extract is dissolved/emulsified in about 5 to 50 ml of the one or more liquids per gram of the crude extract.

57. The imprinted polymer, imprinted polymer bead or methods of any one of the preceding claims wherein the method of producing an enriched cannabinoid extract from a crude Cannabis extract further comprises a rinse step following step (a) and prior to step (b) of washing the imprinted polymer with a rinse liquid to remove at least a portion of undesired components from the crude extract.

58. The imprinted polymer, imprinted polymer bead or methods of any one of the preceding claims wherein where steps (a) and (b) are repeated, the rinse step is optionally repeated one or more times.

59. The imprinted polymer, imprinted polymer bead or methods of any one of the preceding claims wherein the rinse liquid comprises one or more of water, ethanol, methanol, ethyl acetate, isopropyl alcohol, acetonitrile, acetone, tetrahydrofuran (THF).

60. The imprinted polymer, imprinted polymer bead or methods of any one of the preceding claims wherein step (b) is followed by collection of the enriched Cannabis extract that that has been produced by eluting the cannabinoids from the imprinted polymer.

61. The imprinted polymer, imprinted polymer bead or methods of any one of the preceding claims wherein the enriched Cannabis extract is used in step (a) in place of the crude Cannabis extract. the enriched cannabinoid extract from step (b) used in place of the crude Cannabis extract in step (a) and step (a) and step (b) are repeated using the enriched cannabinoid extract, optionally the eluent is reduced to a desired volume or increased to a desired volume prior to repeating step (b).

62. The imprinted polymer, imprinted polymer bead or methods of any one of the preceding claims wherein the elution solvent comprises one or more of ethanol, methanol, ethyl acetate, isopropyl alcohol, acetonitrile, acetone or THF, optionally the elution solvent is mixture of more than one solvent, optionally the proportions of the solvents change over the course of step (b), preferably the elution solvent comprises ethanol, preferably the elution solvent consists of ethanol.

63. The imprinted polymer, imprinted polymer bead or methods of any one of the preceding claims wherein the step of eluting the cannabinoids from the imprinted polymer with an elution solvent comprises collecting the eluent in one or more than one portion.

64. The imprinted polymer, imprinted polymer bead or methods of any one of the preceding claims wherein the method of producing an enriched cannabinoid extract from a crude Cannabis extract further comprises the step following step (b) of regenerating the imprinted polymer using a regeneration solvent, optionally the regeneration solvent comprises one or more of isopropyl alcohol, acetone, an alkane (for example hexane).

65. The imprinted polymer, imprinted polymer bead or methods of any one of the preceding claims wherein the crude Cannabis extract is produced by extraction with supercritical carbon dioxide, subcritical carbon dioxide, ethanol, one or more hydrocarbons (for example propane, butane, hexane).

66. The imprinted polymer, imprinted polymer bead or methods of any one of the preceding claims wherein the crude Cannabis extract comprises a substantial proportion of at least one non-cannabinoid material selected from one or more of lipids (including waxes, fats, wax esters), plant pigments, glycerides, unsaturated fatty acids, pesticide contaminants, heavy metal contaminants, terpenes, carotenes, flavonoids.

67. The imprinted polymer, imprinted polymer bead or methods of any one of the preceding claims wherein the crude Cannabis extract comprises at least 1% fat or wax, optionally crude Cannabis extract comprises at least 2% fat or wax, optionally the crude Cannabis extract comprises at least 3% fat or wax, optionally the crude Cannabis extract comprises at least 4% fat or wax, optionally the crude Cannabis extract comprises at least 5% fat or wax, optionally the crude Cannabis extract comprises about 1% to 60% fat or wax, optionally the crude Cannabis extract comprises about 2% to 60% fat or wax, optionally the crude Cannabis extract comprises about 3% to 60% fat or wax, optionally the crude Cannabis extract comprises about 4% to 60% fat or wax, optionally the crude Cannabis extract comprises about 5% to 60% fat or wax, optionally the crude Cannabis extract comprises about 1% to 50% fat or wax, optionally the crude Cannabis extract comprises about 2% to 50% fat or wax, optionally the crude Cannabis extract comprises about 3% to 50% fat or wax, optionally the crude Cannabis extract comprises about 4% to 50% fat or wax, optionally the crude Cannabis extract comprises about 5% to 50% fat or wax, optionally the crude Cannabis extract is not winterized.

68. An enriched cannabinoid extract produced by the method of any one of claims 1 to 67.

69. An enriched cannabinoid extract comprising: greater than 65% combined mass of one or more cannabinoid(s), at least one cannabinoid selected from the group CBD, THC, CBN, CBND, CBC, THCV, CBL, CBE and CBDV; greater than about 0.03% by weight CBG; and at least one terpene, selected from linalool, caryophyllene oxide, guaiol, alpha-bisabolol, beta-caryophyllene, alpha-humulene, trans-nerolidol.

70. The enriched cannabinoid extract of claim 68 or 69 wherein the extract comprises less than 10% lipids, preferably the enriched cannabinoid extract comprises less than 5% lipids, preferably the enriched cannabinoid extract comprises less than 1% lipids, preferably the enriched cannabinoid extract comprises less than 0.05% lipids, preferably the enriched cannabinoid extract comprises less than 0.05% lipids, preferably the enriched cannabinoid extract comprises less than 0.01% lipids, preferably the enriched cannabinoid extract comprises substantially no lipids.

71. The enriched cannabinoid extract of any one of claims 68 to 70 wherein the enriched cannabinoid extract comprises less than 0.1% by mass pesticide residue, preferably the enriched cannabinoid extract comprises less than 0.05% by mass pesticide residue, preferably the enriched cannabinoid extract comprises less than 0.01% by mass pesticide residue, preferably the enriched cannabinoid extract is substantially free from pesticide residue.

72. The enriched cannabinoid extract of any one of claims 68 to 71 wherein the enriched cannabinoid extract comprises less than 0.1% by mass heavy metals, preferably the enriched cannabinoid extract comprises less than 0.05% by mass heavy metals, preferably the enriched cannabinoid extract comprises less than 0.01% by mass heavy metals, preferably the enriched cannabinoid extract comprises less than 0.001% by mass heavy metals, preferably the enriched cannabinoid extract is substantially free from heavy metals.

73. The enriched cannabinoid extract of any one of claims 68 to 72 wherein the enriched cannabinoid extract comprises less than about 2% by mass non-cannabinoid molecules that cause bitter taste, preferably the enriched cannabinoid extract comprises less than about 1% by mass non-cannabinoid molecules that cause bitter taste, preferably the enriched cannabinoid extract comprises less than about 0.5% by mass non-cannabinoid molecules that cause bitter taste, preferably the enriched cannabinoid extract comprises less than about 0.1% by mass non-cannabinoid molecules that cause bitter taste, preferably the enriched cannabinoid extract comprises less than about 0.1% by mass non-cannabinoid molecules that cause bitter taste, preferably the enriched cannabinoid extract is substantially free from non-cannabinoid molecules that cause bitter taste.

74. The enriched cannabinoid extract of any one of claims 68 to 73 wherein the enriched cannabinoid extract comprises less than 30%, preferably less than 25%, preferably less than 15% content by weight of molecules found within Cannabis plant material that fall outside the size range of about 100 to 450 grams per mol.

75. The enriched cannabinoid extract of any one of claims 68 to 74 wherein the enriched cannabinoid extract comprises greater than 1.15% by weight CBG, preferably the enriched cannabinoid extract comprises greater than 2% by weight CBG, preferably the enriched cannabinoid extract comprises between about 2-10% by weight CBG, preferably the enriched cannabinoid extract comprises between about 2-5% by weight CBG.

76. The enriched cannabinoid extract of any one of claims 68 to 75 wherein the enriched cannabinoid extract comprises greater than about 70% combined mass of one or more cannabinoid(s), preferably the enriched cannabinoid extract comprises greater than about 75% combined mass of one or more cannabinoid(s), preferably the enriched cannabinoid extract comprises greater than about 80% combined mass of one or more cannabinoid(s).

77. The enriched cannabinoid extract of any one of claims 68 to 76 wherein the enriched cannabinoid extract comprises at least one cannabinoid acid selected from the group CBDA, THCA, CBGA, CBCA, CBLA, CBEA-A and CBEA-B, preferably the enriched cannabinoid extract comprises at least two cannabinoid acids selected from the group CBDA, THCA, CBGA, CBCA, CBLA, CBEA-A and CBEA-B, preferably the enriched cannabinoid extract comprises CBDA.

78. The enriched cannabinoid extract of any one of claims 68 to 77 wherein the enriched cannabinoid extract comprises more than one terpene.

79. The enriched cannabinoid extract of any one of claims 68 to 78 wherein the enriched cannabinoid extract comprises linalool or caryophyllene oxide or guaiol or alpha-bisabolol or beta-caryophyllene or alpha-humulene or trans-nerolidol.

1. An imprinted polymer for producing an enriched cannabinoid extract from a crude Cannabis extract,
wherein the polymer is imprinted with a template organic molecule with a molecular weight of about 150 to 450 grams per mol comprising a hydroxyphenyl group, and
wherein the polymer has been prepared from one or more polymerizable monomers.

2. The imprinted polymer of claim 1 wherein the one or more polymerizable monomer(s) is selected from an acryl or a vinyl monomer.

3. The imprinted polymer of claim 2 wherein the acryl monomer is selected from acrylic acid, tert-butylacrylamide, N-phenylacrylamide, N-methylacrylamide, methacrylic acid, 2-(hydroxyethyl)methacrylate, ethylene glycol dimethacrylate (EGDMA), methacrylic anhydride or trimethylolpropane trimethacrylate,

4. The imprinted polymer of any one of the preceding claims wherein the polymer has been prepared from one or more acryls monomer(s).

5. The imprinted polymer of any one of the preceding claims wherein the polymer has been prepared from one acryl monomer as the only monomer.

6. The imprinted polymer of any one of the preceding claims wherein the polymer has been prepared from ethylene glycol dimethacrylate (EGDMA) as the only monomer.

7. The imprinted polymer of any one of claims 1 to 5 wherein the polymer has been prepared from ethylene glycol dimethacrylate (EGDMA) and tert-butylacryamide (TBA) monomers.

8. The imprinted polymer of claim 2 wherein the vinyl monomer is selected from a styryl or vinylpyridine.

9. The imprinted polymer of claim 8 wherein the polymer has been prepared from divinylbenzene (DVB) and styrene monomers.

10. The imprinted polymer of any one of the preceding claims wherein the template comprises the structure: 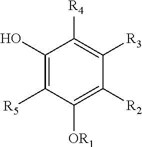
wherein
R₁ is H
R₂ is selected from H, or an organic group,
R₃ is selected from H, —OH, or an organic group,
R₄ is H or an organic group,
R₅ is H, or an organic group,
or R₁ and R₂ together form a 5- or 6-membered ring, preferably a 6-membered ring, optionally substituted with one or more of —OH, or an organic group, or
or R₁ and R₅ together form a 5- or 6-membered ring, optionally substituted with one or more substitutes independently selected from —OH, or an organic group, and
wherein the 5- or 6-membered ring is optionally fused to a further ring which may be optionally substituted with one or more alkyl.

11. The imprinted polymer of claim 10 wherein one or more of the R₁, R₂, R₃, R₄ or R₅ groups is a saturated alkyl group, preferably a saturated alkyl group with 2 to 8 carbons.

12. The imprinted polymer of claim 10 or 11 wherein at least one of R₂, R₃, and R₅ are an alkyl, preferably a 4-6 alkyl.

13. The imprinted polymer of any one of claims 10 to 12 wherein
R₁ is H
R₂ is selected from H, alkyl, or CORₓ, wherein Rₓ is alkyl, cycloalkyl or aryl, preferably aryl,
R₃ is selected from H, —OH or alkyl,
R₄ is H or alkyl,
R₅ is H, alkyl, or cycloalkyl optionally substituted with one or more alkyl or —OH,
or R₁ and R₂ together form a 5- or 6-membered ring, optionally substituted with one or more substituents independently selected from —OH, alkyl, or aryl, wherein the aryl is optionally substituted with one or more —OH,
or R₁ and R₅ together form a 5- or 6-membered ring, optionally substituted with one or more substituents independently selected from —OH, alkyl, or aryl, wherein the aryl is optionally substituted with one or more —OH, and wherein the 5- or 6-membered ring is optionally fused to a further 5- or 6-membered ring, which may be optionally substituted with one or more independently selected alkyl.

14. The imprinted polymer of any one of the preceding claims wherein the template molecule is a cannabinoid.

15. The imprinted polymer of any one of the preceding claims wherein the template is Cannabidiol (CBD).

16. The imprinted polymer of any one of claims 1 to 13 wherein the template molecule is a flavan-3-ol.

17. The imprinted polymer of any one of claims 1 to 13 or 16 wherein the template molecule is catechin.

18. A method of making an imprinted polymer for producing an enriched cannabinoid extract from a crude Cannabis extract, the method comprising polymerizing one or more polymerizable monomer(s) in the presence of a template organic molecule with a molecular weight of about 150 to 450 grams per mol comprising a hydroxyphenyl group, and optionally subsequently at least partially removing the template molecule from the imprinted polymer.

19. A method of producing an enriched cannabinoid extract from a crude Cannabis extract, the method comprising the steps of:
a) contacting an imprinted polymer with the crude Cannabis extract, wherein the imprinted polymer is a polymer that has been imprinted with a template organic molecule with a molecular weight of about 150 to 450 grams per mol comprising a hydroxyphenyl group, and wherein the polymer has been prepared from one or more polymerizable monomers;
b) eluting the cannabinoids from the imprinted polymer with an elution solvent to produce the enriched cannabinoid extract comprising one or more cannabinoid(s).

20. The method of claim 19 wherein the crude Cannabis extract comprises at least 3% fat or wax, optionally the crude Cannabis extract is not winterized.

21. The method of claim 19 or 20 wherein the method further comprises the step prior to step (a) of forming an emulsion and/or dissolving the crude Cannabis extract in one or more liquid(s) to give an emulsion and/or solution of crude Cannabis extract, such that the imprinted polymer is contacted with the crude Cannabis extract in the form of an emulsion and/or solution.

22. The method of any one of claims 19 to 21 wherein the method further comprises a rinse step following step (a) and prior to step (b) of washing the imprinted polymer with a rinse liquid to remove at least a portion of undesired components from the crude extract.

23. The method of any one of claims 19 to 22 wherein step (b) is followed by collection of the enriched Cannabis extract, and the enriched Cannabis extract is used in step (a) in place of the crude Cannabis extract.

24. The method of any one of claims 19 to 23 wherein the step of eluting the cannabinoids from the imprinted polymer with an elution solvent comprises collecting the eluent in one or more than one portion.

25. The method of any one of claims 19 to 24 wherein the enriched cannabinoid extract has an increased proportion of CBD and/or the enriched cannabinoid extract has an increased proportion of CBG and/or the enriched cannabinoid extract has an increased proportion of THC.

26. The method of any one of claims 19 to 25 wherein the enriched cannabinoid extract comprises a cannabinoid in acid form.

27. The method of any one of claims 19 to 26 wherein the enriched cannabinoid extract has a reduced proportion of heavy metals and/or pesticides compared to the crude Cannabis extract.

28. The method of any one of claims 19 to 27 wherein the enriched cannabinoid extract has a reduced proportion of lipid compared to the crude Cannabis extract.

29. The methods of any one of claims 19 to 28 wherein the proportion of at least one terpene is increased compared to the crude Cannabis extract.

30. An enriched cannabinoid extract comprising:
greater than 65% combined mass of one or more cannabinoid(s),
at least one cannabinoid selected from the group CBD, THC, CBN, CBND, CBC, THCV, CBL, CBE and CBDV;
greater than about 0.03% by weight CBG; and
at least one terpene, selected from linalool, caryophyllene oxide, guaiol, alpha-bisabolol, beta-caryophyllene, alpha-humulene, trans-nerolidol.

31. The enriched cannabinoid extract of claim 30 wherein the enriched cannabinoid extract comprises less than 0.1% by mass pesticide residue.

32. The enriched cannabinoid extract of claim 30 or 31 wherein the enriched cannabinoid extract comprises less than 0.1% by mass heavy metals.

33. The enriched cannabinoid extract of any one of claims 30 to 32 wherein the enriched cannabinoid extract comprises greater than about 70% combined mass of one or more cannabinoid(s)

34. The enriched cannabinoid extract of any one of claims 30 to 34 wherein the enriched cannabinoid extract comprises more than one terpene.
